(19) United States
(12) Patent Application Publication
(10) Pub. No.: US 2024/0368185 A1
(43) Pub. Date: Nov. 7, 2024

(54) MACROCYCLIC AZOLOPYRIDINES

(71) Applicant: Fulcrum Therapeutics, Inc., Cambridge, MA (US)

(72) Inventors: Steven M. Mennen, Boston, MA (US); Robert Alex Mayo, Halifax (CA)

(21) Appl. No.: 18/553,317

(22) PCT Filed: Mar. 31, 2022
(86) PCT No.: PCT/US2022/022893

§ 371 Date: Sep. 29, 2023

Related U.S. Application Data
(60) Provisional application No. 63/170,212, filed on Apr. 2, 2021.

(51) Int. Cl. C07D 498/22

(52) U.S. Cl. CPC C07D 498/22

(57) ABSTRACT

The disclosure is in part directed to crystalline forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, and crystalline salt forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

### CROSS-REFERENCE

This application claims priority to U.S. Provisional Application No. 63/170,212 filed Apr. 2, 2021, which is incorporated herein by reference in its entirety.

### BACKGROUND

Polycomb Repressive Complex 2 (PRC2) is a multi-subunit chromatin regulatory complex that functions in repression of gene expression and is dysregulated in many human diseases. PRC2 includes SUZ12 (suppressor of zeste 12), EED (embryonic ectoderm development) and the catalytic subunit, EZH2 (enhancer of zeste homolog 2), and represses genes by methylating histone H3 on lysine 27 (H3K27me3) at and around the promoter regions of genes. EED mediates repression of gene activity by binding to the H3K27me3 mark where it allosterically activates the methyltransferase activity of PRC2 (e.g., trimethylation of lysine 27 on histone H3 (H3K27me3). This critical component of chromatin regulation is involved in modulation of gene transcription and plays a key role in development, differentiation, and regeneration.

EED regulates PRC2 in the silencing of expression of genes and gene clusters involved in development, e.g., fetal orthologues (e.g., gamma globin), Hox genes, and in X chromosome inactivation. Aberrant expression of PRC2 has also been observed in various human cancers, for example, hepatocellular carcinoma, breast cancer, and prostate cancer. Thus, EED and/or PRC2 provides a pharmacological target for the treatment of diseases or disorders, for example, cancers and blood disorders, to impact transcription of specific target genes in, for example, blood and other tissues.

Polymorphism is the ability of a substance to crystallize in more than one crystal lattice arrangement. Crystallization, or polymorphism, can influence many aspects of the solid-state properties of a drug substance. A crystalline substance may differ considerably from an amorphous form, and different crystal modifications of a substance may differ considerably from one another in many respects including solubility, dissolution rate and/or bioavailability. Generally, it is difficult to predict whether a given compound will form any crystalline solid-state forms. It is even more difficult to predict the physical properties of these crystalline solid-state forms. Further, it can be advantageous to have a crystalline form of a therapeutic agent for certain formulations and/or for manufacturing processes.

### SUMMARY

The present disclosure is directed, at least in part, to crystalline forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

For example, disclosed herein is a crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, characterized by a powder X-ray diffraction pattern having a characteristic peak in degrees 2θ at about 7.6, for example, characterized by a powder X-ray diffraction pattern having characteristic peaks in degrees 2θ at about 7.6, 11.9, and 15.3, for example, characterized by a powder X-ray diffraction pattern having characteristic peaks in degrees 2θ at about 7.6, 11.9, 14.5, 15.3, 20.7, and 22.6, for example, characterized by a powder X-ray diffraction pattern having characteristic peaks in degrees 2θ at about 7.6, 11.9, 14.5, 15.3, 16.1, 17.2, 17.3, 20.7, 22.6, 23.3, 26.2, and 24.5.

Also disclosed herein are pharmaceutically acceptable, crystalline salt forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. For example, a disclosed crystalline salt form may be selected from the group consisting of a benzenesulfonic acid salt, a citric acid salt, a fumaric acid salt, a hydrochloride salt, a maleic acid salt, a L-malic acid salt, a methanesulfonic acid salt, a phosphoric acid salt, a pyruvic acid salt, a sulfuric acid salt, a L-tartaric acid salt, and a toluenesulfonic acid salt, and crystalline hydrates and solvates thereof.

(S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is, for example, a modulator of EED and/or a modulator of PRC2, and is represented by:

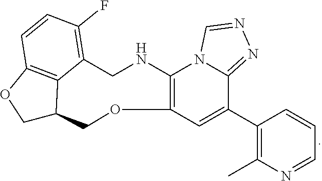 [SMILES: Cc1ncccc1-c1cc2c(n3cnnc13)NCc1c(F)ccc3c1[C@H](CO3)CO2]

Further contemplated herein is a pharmaceutical composition comprising a disclosed crystalline free base form, or a disclosed crystalline salt form, of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient, for example, a composition that is formulated for oral, subcutaneous or intravenous administration. Further contemplated herein is a drug substance comprising at least a detectable amount of a disclosed crystalline free base form, or a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. For example, disclosed herein is a drug substance comprising substantially pure crystalline free base form or a crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

Also provided herein is a method of treating a blood disorder (e.g., sickle cell disease or β-thalassemia) in a patient in need thereof, comprising administering to the patient an effective amount of a disclosed crystalline free base form, or a disclosed crystalline salt form, of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. For example, provided herein is a method of treating a blood disorder (e.g., sickle cell disease or β-thalassemia) in a patient in need thereof, comprising administering to the patient an effective amount of a pharmaceutical composition comprising a disclosed crystalline free base form, or a pharmaceutical composition comprising a disclosed crystalline salt form, of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

Further provided herein is a method of treating a cancer in a patient in need thereof, comprising administering to the patient an effective amount of a disclosed crystalline free base form, or a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. For example, provided herein is a method of treating a cancer in a patient in need thereof, comprising administering to the patient an effective amount of a pharmaceutical composition comprising a disclosed crystalline free base form, or a pharmaceutical composition comprising disclosed crystalline salt form, of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

### BRIEF DESCRIPTION OF THE DRAWINGS

FIG. 1 depicts the X-ray powder diffraction (XRPD) pattern of Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base.

FIG. 2 depicts the characterization of Form P by differential scanning calorimetry (DSC).

FIG. 3 depicts the X-ray powder diffraction (XRPD) pattern of Form A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 4 depicts the X-ray powder diffraction (XRPD) pattern of Form B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 5 depicts the X-ray powder diffraction (XRPD) pattern of Form C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 6 depicts the X-ray powder diffraction (XRPD) pattern of Form D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 7 depicts the X-ray powder diffraction (XRPD) pattern of Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate.

FIG. 8 depicts the characterization of Form E by differential scanning calorimetry (DSC).

FIG. 9 depicts the X-ray powder diffraction (XRPD) pattern of Form F of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 10 depicts the X-ray powder diffraction (XRPD) pattern of Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate.

FIG. 11 depicts the characterization of Form H by differential scanning calorimetry (DSC).

FIG. 12 depicts the X-ray powder diffraction (XRPD) pattern of Form I of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 13 depicts the X-ray powder diffraction (XRPD) pattern of Form J of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 14 depicts the X-ray powder diffraction (XRPD) pattern of Form K of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 15 depicts the characterization of Form K by differential scanning calorimetry (DSC).

FIG. 16 depicts the X-ray powder diffraction (XRPD) pattern of Form L of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 17 depicts the X-ray powder diffraction (XRPD) pattern of Form M of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 18 depicts the X-ray powder diffraction (XRPD) pattern of Form N of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 19 depicts the X-ray powder diffraction (XRPD) pattern of Form O of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 20 depicts the characterization of Form O by differential scanning calorimetry (DSC).

FIG. 21 depicts the X-ray powder diffraction (XRPD) pattern of Form Q of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 22 depicts the X-ray powder diffraction (XRPD) pattern of Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 23 depicts the characterization of Form R by differential scanning calorimetry (DSC).

FIG. 24 depicts the X-ray powder diffraction (XRPD) pattern of Form S of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 25 depicts the X-ray powder diffraction (XRPD) pattern of Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 26 depicts the characterization of Form T by differential scanning calorimetry (DSC).

FIG. 27 depicts the X-ray powder diffraction (XRPD) pattern of Form U of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 28 depicts the X-ray powder diffraction (XRPD) pattern of Form V of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 29 depicts the X-ray powder diffraction (XRPD) pattern of Form W of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

FIG. 30 depicts the X-ray powder diffraction (XRPD) pattern of Form 1-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt.

FIG. 31 depicts the X-ray powder diffraction (XRPD) pattern of Form 1-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt.

FIG. 32 depicts the characterization of Form 1-B by differential scanning calorimetry (DSC).

FIG. 33 depicts the X-ray powder diffraction (XRPD) pattern of Form 2-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt.

FIG. 34 depicts the X-ray powder diffraction (XRPD) pattern of Form 2-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt.

FIG. 35 depicts the X-ray powder diffraction (XRPD) pattern of Form 2-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt.

FIG. 36 depicts the characterization of Form 2-C by differential scanning calorimetry (DSC).

FIG. 37 depicts the X-ray powder diffraction (XRPD) pattern of Form 3-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, fumaric acid salt.

FIG. 38 depicts the characterization of Form 3-A by differential scanning calorimetry (DSC).

FIG. 39 depicts the X-ray powder diffraction (XRPD) pattern of Form 5-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt.

FIG. 40 depicts the X-ray powder diffraction (XRPD) pattern of Form 5-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt.

FIG. 41 depicts the characterization of Form 5-B by differential scanning calorimetry (DSC).

FIG. 42 depicts the X-ray powder diffraction (XRPD) pattern of Form 5-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt.

FIG. 43 depicts the characterization of Form 5-C by differential scanning calorimetry (DSC).

FIG. 44 depicts the X-ray powder diffraction (XRPD) pattern of Form 5-D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt.

FIG. 45 depicts the X-ray powder diffraction (XRPD) pattern of Form 7-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt.

FIG. 46 depicts the characterization of Form 7-A by differential scanning calorimetry (DSC).

FIG. 47 depicts the X-ray powder diffraction (XRPD) pattern of Form 8-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt.

FIG. 48 depicts the characterization of Form 8-A by differential scanning calorimetry (DSC).

FIG. 49 depicts the X-ray powder diffraction (XRPD) pattern of Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt.

FIG. 50 depicts the characterization of Form 8-B by differential scanning calorimetry (DSC).

FIG. 51 depicts the X-ray powder diffraction (XRPD) pattern of Form 9-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt.

FIG. 52 depicts the X-ray powder diffraction (XRPD) pattern of Form 9-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt.

FIG. 53 depicts the characterization of Form 9-B by differential scanning calorimetry (DSC).

FIG. 54 depicts the X-ray powder diffraction (XRPD) pattern of Form 9-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt.

FIG. 55 depicts the X-ray powder diffraction (XRPD) pattern of Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt.

FIG. 56 depicts the characterization of Form 10-A by differential scanning calorimetry (DSC).

FIG. 57 depicts the X-ray powder diffraction (XRPD) pattern of Form 11-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, pyruvic acid salt.

FIG. 58 depicts the characterization of Form 11-A by differential scanning calorimetry (DSC).

FIG. 59 depicts the X-ray powder diffraction (XRPD) pattern of Form 12-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, sulfuric acid salt.

FIG. 60 depicts the characterization of Form 12-A by differential scanning calorimetry (DSC).

FIG. 61 depicts the X-ray powder diffraction (XRPD) pattern of Form 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt.

FIG. 62 depicts the characterization of Form 13-A by differential scanning calorimetry (DSC).

FIG. 63 depicts the X-ray powder diffraction (XRPD) pattern of Form 13-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt.

FIG. 64 depicts the characterization of Form 13-B by differential scanning calorimetry (DSC).

FIG. 65 depicts the X-ray powder diffraction (XRPD) pattern of Form 14-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt.

FIG. 66 depicts the X-ray powder diffraction (XRPD) pattern of Form 14-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt.

FIG. 67 depicts the X-ray powder diffraction (XRPD) pattern of amorphous (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base. Line (1) depicts Form E material, and line (2) depicts amorphous material after evaporation of THF from Form E under atmosphere at 50° C. and further drying of the residue under vacuum at 50° C.

FIG. 68 depicts the X-ray powder diffraction (XRPD) pattern of various solid forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine obtained from crystallization from various solvents.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “crystalline form” refers to a crystal form or modification that can be characterized by analytical methods such as, e.g., X-ray powder diffraction (XRPD) and/or Differential scanning calorimetry (DSC). The crystalline compounds disclosed herein can exist in solvated as well as unsolvated forms with solvents such as water, ethanol, and the like. Unless otherwise indicated or inferred, it is intended that disclosed crystalline compounds include both solvated and unsolvated forms.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable excipient” or “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment, for example, of a cancer or a blood disorder is desired. “Modulation” includes antagonism (e.g., inhibition), agonism, partial antagonism and/or partial agonism.

In the present specification, the terms “effective amount” or “therapeutically effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of basic groups that may be present in compounds used in the compositions. Compounds included in the present compositions that are basic in nature are capable of forming a wide variety of salts with various inorganic and organic acids.

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. The term “about” in the context of peaks at degrees 26 means that there is an uncertainty in the measurements of the 2θ of ±0.5 (expressed in 20) or that there is an uncertainty in the measurements of the 2θ of ±0.2 (expressed in 20).

### Crystalline Forms

The present disclosure is directed, at least in part, to crystalline forms of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

For example, disclosed herein is a crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, characterized by a powder X-ray diffraction pattern having a characteristic peak in degrees 26 at about 7.6 (referred to herein as “Form P”).

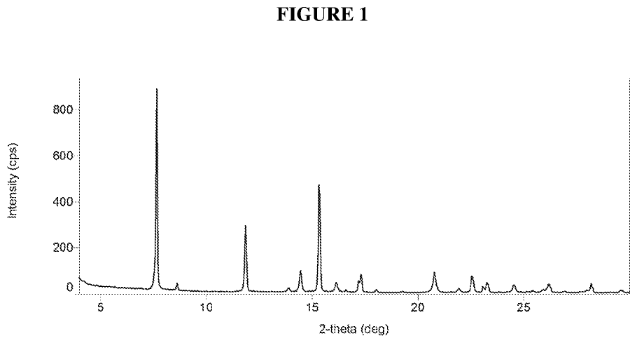

In one embodiment, the crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, is characterized by a powder X-ray diffraction pattern that has a characteristic peak in degrees 26 at about 11.9, is characterized by a powder X-ray diffraction pattern that has a characteristic peak in degrees 26 at about 14.5, is characterized by a powder X-ray diffraction pattern that has a characteristic peak in degrees 26 at about 15.3, is characterized by a powder X-ray diffraction pattern that has a characteristic peak in degrees 26 at about 16.1, is characterized by a powder X-ray diffraction pattern that has a characteristic peak in degrees 2θ at about 17.2, is characterized by a powder X-ray diffraction pattern that has a characteristic peak in degrees 2θ at about 17.3, is characterized by a powder X-ray diffraction pattern that has a characteristic peak in degrees 2θ at about 20.7, is characterized by a powder X-ray diffraction pattern that has a characteristic peak in degrees 2θ at about 22.6, is characterized by a powder X-ray diffraction pattern that has a characteristic peak in degrees 2θ at about 23.3, is characterized by a powder X-ray diffraction pattern that has a characteristic peak in degrees 2θ at about 26.2, and/or is characterized by a powder X-ray diffraction pattern that has a characteristic peak in degrees 2θ at about 24.5. In another embodiment, crystalline Form P is characterized by a powder X-ray diffraction pattern having at least one or more characteristic peaks in degrees 2θ at about 7.6, 11.9, and 15.3. In a further embodiment, crystalline Form P is characterized by a powder X-ray diffraction pattern having at least one or more characteristic peaks in degrees 2θ at about 7.6, 11.9, 14.5, 15.3, 20.7, and 22.6. In yet another embodiment, crystalline Form P is characterized by a powder X-ray diffraction pattern having at least one or more characteristic peaks in degrees 2θ at about 7.6, 11.9, 14.5, 15.3, 16.1, 17.2, 17.3, 20.7, 22.6, 23.3, 26.2, and 24.5. For example, a contemplated crystalline form has a powder X-ray diffraction pattern shown in FIG. 1. In one embodiment, the powder X-ray diffraction pattern of the crystalline form was obtained using Cu Kα radiation.

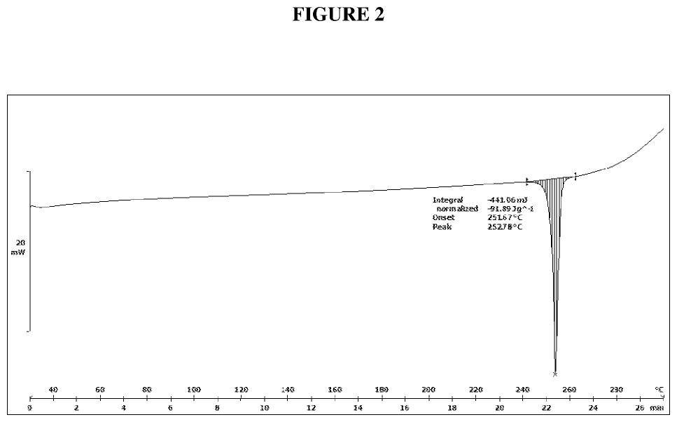

The contemplated crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, may be characterized by a differential scanning calorimetry (DSC) profile showing a characteristic endotherm with an onset of about 252° C. and a peak of about 253° C. Form P, for example, may be characterized by the differential scanning calorimetry profile shown in FIG. 2.

The contemplated crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, may be characterized by a thermogravimetric analysis (TGA) profile showing a mass loss of about 0.46 wt. % up to about 260° C. In some embodiments, crystalline Form P may be characterized by a dynamic vapor sorption (DVS) profile showing a reversable total mass change of about 0.53 wt. % between about 2 to about 92% relative humidity (RH) at 25° C. In other embodiments, crystalline Form P may be characterized by optical microscopy showing a rod-like and/or a plate-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.5 (referred to herein as “Form A”), is disclosed herein.

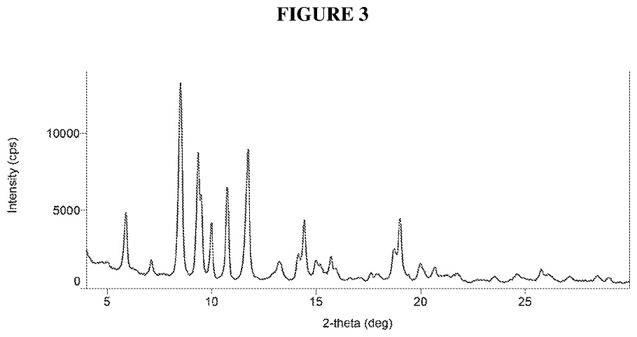

In one embodiment, the crystalline Form A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 5.9, is characterized by a powder X-ray diffraction pattern that has a characteristic peak in degrees 2θ at about 9.3, is characterized by a powder X-ray diffraction pattern that has a characteristic peak in degrees 2θ at about 9.5, is characterized by a powder X-ray diffraction pattern that has a characteristic peak in degrees 2θ at about 10.0, is characterized by a powder X-ray diffraction pattern that has a characteristic peak in degrees 2θ at about 10.7, is characterized by a powder X-ray diffraction pattern that has a characteristic peak in degrees 2θ at about 11.7, is characterized by a powder X-ray diffraction pattern that has a characteristic peak in degrees 2θ at about 14.4, is characterized by a powder X-ray diffraction pattern that has a characteristic peak in degrees 2θ at about 18.7, is characterized by a powder X-ray diffraction pattern that has a characteristic peak in degrees 2θ at about 19.0, is characterized by a powder X-ray diffraction pattern that has a characteristic peak in degrees 2θ at about 25.8, and/or is characterized by a powder X-ray diffraction pattern that has a characteristic peak in degrees 2θ at about 28.4. In yet another embodiment, crystalline Form A is characterized by a powder X-ray diffraction pattern having at least one or more characteristic peaks in degrees 2θ at about 5.9, 8.5, 9.3, 9.5, 10.0, 10.7, 11.7, 14.4, 18.7, 19.0, 25.8, and 28.4. For example, a contemplated crystalline form has a powder X-ray diffraction pattern shown in FIG. 3. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.5 (referred to herein as “Form B”), is disclosed herein.

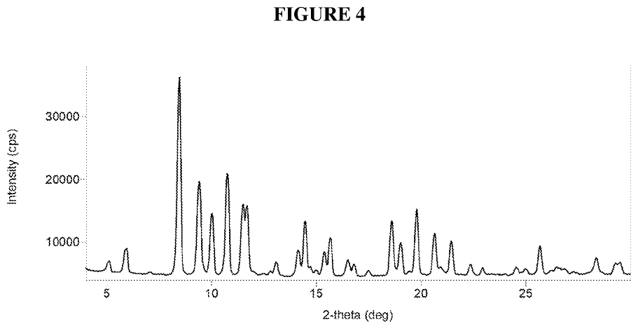

In one embodiment, the crystalline Form B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 5.1, is characterized by a powder X-ray diffraction pattern that has a characteristic peak in degrees 2θ at about 9.4, is characterized by a powder X-ray diffraction pattern that has a characteristic peak in degrees 2θ at about 9.5, is characterized by a powder X-ray diffraction pattern that has a characteristic peak in degrees 2θ at about 10.0, is characterized by a powder X-ray diffraction pattern that has a characteristic peak in degrees 2θ at about 10.7, is characterized by a powder X-ray diffraction pattern that has a characteristic peak in degrees 2θ at about 11.5, is characterized by a powder X-ray diffraction pattern that has a characteristic peak in degrees 2θ at about 11.7, is characterized by a powder X-ray diffraction pattern that has a characteristic peak in degrees 2θ at about 14.4, is characterized by a powder X-ray diffraction pattern that has a characteristic peak in degrees 2θ at about 15.7, is characterized by a powder X-ray diffraction pattern that has a characteristic peak in degrees 2θ at about 18.6, is characterized by a powder X-ray diffraction pattern that has a characteristic peak in degrees 2θ at about 20.6, and/or is characterized by a powder X-ray diffraction pattern that has a characteristic peak in degrees 2θ at about 21.4. In yet another embodiment, crystalline Form B is characterized by a powder X-ray diffraction pattern having at least one or more characteristic peaks in degrees 2θ at about 5.1, 8.5, 9.4, 10.0, 10.7, 11.5, 11.7, 14.4, 15.7, 18.6, 19.8, 20.6, and 21.4. For example, a contemplated crystalline form has a powder X-ray diffraction pattern shown in FIG. 4. In one embodiment, the powder X-ray diffraction pattern of the crystalline form was obtained using Cu Kα radiation. In other embodiments, crystalline Form B may be characterized by optical microscopy showing a hair-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 9.0 (referred to herein as “Form C”), is disclosed herein.

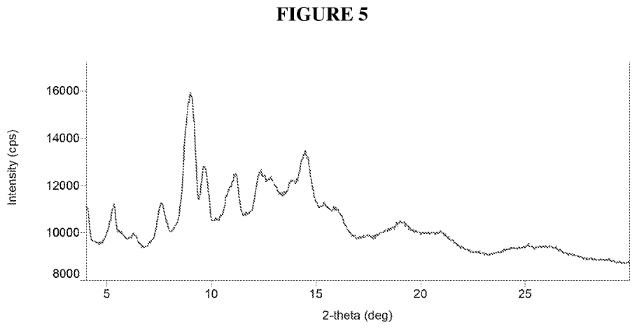

In one embodiment, the crystalline Form C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 4.0, is characterized by a powder X-ray diffraction pattern that has a characteristic peak in degrees 2θ at about 5.3, is characterized by a powder X-ray diffraction pattern that has a characteristic peak in degrees 2θ at about 7.6, is characterized by a powder X-ray diffraction pattern that has a characteristic peak in degrees 2θ at about 9.7, is characterized by a powder X-ray diffraction pattern that has a characteristic peak in degrees 2θ at about 11.2, is characterized by a powder X-ray diffraction pattern that has a characteristic peak in degrees 2θ at about 12.3, is characterized by a powder X-ray diffraction pattern that has a characteristic peak in degrees 2θ at about 12.9, is characterized by a powder X-ray diffraction pattern that has a characteristic peak in degrees 2θ at about 13.7, is characterized by a powder X-ray diffraction pattern that has a characteristic peak in degrees 2θ at about 14.5, is characterized by a powder X-ray diffraction pattern that has a characteristic peak in degrees 2θ at about 15.9, and/or is characterized by a powder X-ray diffraction pattern that has a characteristic peak in degrees 2θ at about 19.0. In yet another embodiment, crystalline Form C is characterized by a powder X-ray diffraction pattern having at least one or more characteristic peaks in degrees 2θ at about 4.0, 5.3, 7.6, 9.0, 9.7, 11.2, 12.3, 12.9, 13.7, 14.5, 15.9, and 19.0. For example, a contemplated crystalline form has a powder X-ray diffraction pattern shown in FIG. 5. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.6 (referred to herein as “Form D”), is disclosed herein.

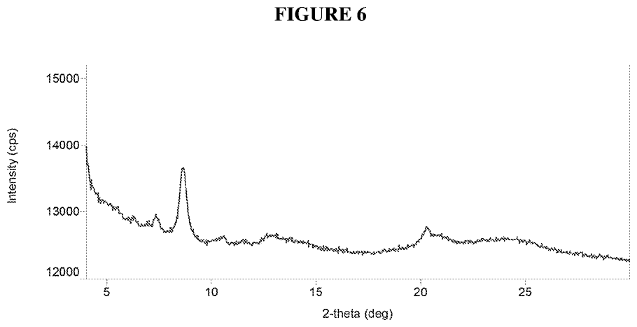

In one embodiment, the crystalline Form D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 7.3, is characterized by a powder X-ray diffraction pattern that has a characteristic peak in degrees 2θ at about 10.6, and/or is characterized by a powder X-ray diffraction pattern that has a characteristic peak in degrees 2θ at about 12.9. In yet another embodiment, crystalline Form D is characterized by a powder X-ray diffraction pattern having at least one or more characteristic peaks in degrees 2θ at about 7.3, 8.6, 10.6, and 12.9. For example, a contemplated crystalline form has a powder X-ray diffraction pattern shown in FIG. 6. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, characterized by a powder X-ray diffraction pattern having a characteristic peak in degrees 2θ at about 4.6 (referred to herein as “Form E”), is disclosed herein.

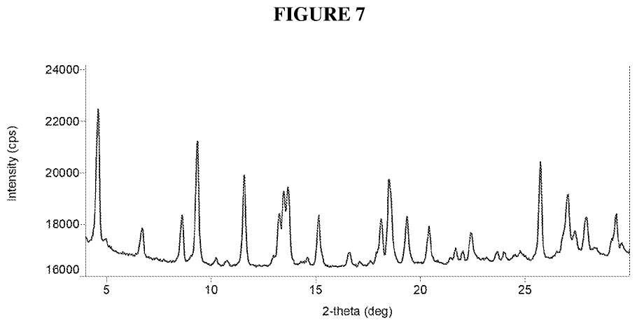

In one embodiment, the crystalline Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, is characterized by a powder X-ray diffraction pattern that has a characteristic peak in degrees 2θ at about 9.3, is characterized by a powder X-ray diffraction pattern that has a characteristic peak in degrees 2θ at about 11.6, is characterized by a powder X-ray diffraction pattern that has a characteristic peak in degrees 2θ at about 13.2, is characterized by a powder X-ray diffraction pattern that has a characteristic peak in degrees 2θ at about 13.5, is characterized by a powder X-ray diffraction pattern that has a characteristic peak in degrees 2θ at about 13.7, is characterized by a powder X-ray diffraction pattern that has a characteristic peak in degrees 2θ at about 15.1, is characterized by a powder X-ray diffraction pattern that has a characteristic peak in degrees 2θ at about 18.1, is characterized by a powder X-ray diffraction pattern that has a characteristic peak in degrees 2θ at about 18.5, is characterized by a powder X-ray diffraction pattern that has a characteristic peak in degrees 2θ at about 19.3, is characterized by a powder X-ray diffraction pattern that has a characteristic peak in degrees 2θ at about 25.7, and/or is characterized by a powder X-ray diffraction pattern that has a characteristic peak in degrees 2θ at about 27.0. In yet another embodiment, crystalline Form E is characterized by a powder X-ray diffraction pattern having at least one or more characteristic peaks in degrees 2θ at about 4.6, 9.3, 11.6, 13.2, 13.5, 13.7, 15.1, 18.1, 18.5, 19.3, 25.7, and 27.0. For example, a contemplated crystalline form has a powder X-ray diffraction pattern shown in FIG. 7. In one embodiment, the powder X-ray diffraction pattern of the crystalline form was obtained using Cu Kα radiation.

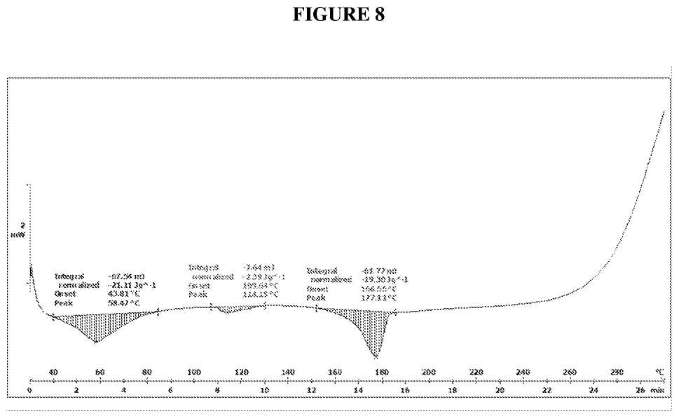

The contemplated crystalline Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, may be characterized by a differential scanning calorimetry (DSC) profile showing a characteristic endotherm with an onset of about 44° C. and a peak of about 58° C., a characteristic endotherm with an onset of about 110° C. and a peak of about 114° C., and a characteristic endotherm with an onset of about 166° C. and a peak of about 177° C. Form E, for example, may be characterized by the differential scanning calorimetry profile shown in FIG. 8.

The contemplated crystalline Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, may be characterized by a thermogravimetric analysis (TGA) profile showing a mass loss of about 5.1 wt. % up to about 170° C. In some embodiments, crystalline Form E may be characterized by a dynamic vapor sorption (DVS) profile showing a reversable total mass change of about 10.3 wt. % between about 2 to about 92% relative humidity (RH) at 25° C. In other embodiments, crystalline Form E may be characterized by optical microscopy showing a hair-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 4.5 (referred to herein as “Form F”), is disclosed herein.

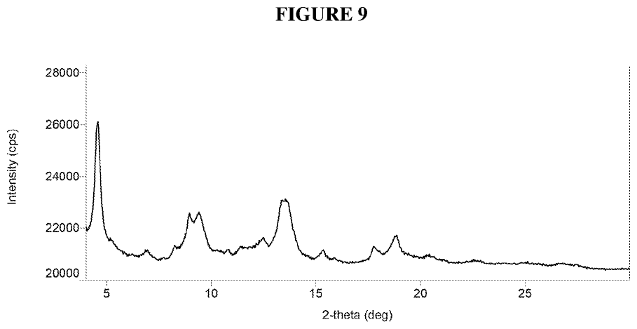

In one embodiment, the crystalline Form F of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 7.0, is characterized by a powder X-ray diffraction pattern that has a characteristic peak in degrees 2θ at about 8.2, is characterized by a powder X-ray diffraction pattern that has a characteristic peak in degrees 2θ at about 9.0, is characterized by a powder X-ray diffraction pattern that has a characteristic peak in degrees 2θ at about 9.3, is characterized by a powder X-ray diffraction pattern that has a characteristic peak in degrees 2θ at about 12.5, is characterized by a powder X-ray diffraction pattern that has a characteristic peak in degrees 2θ at about 13.5, is characterized by a powder X-ray diffraction pattern that has a characteristic peak in degrees 2θ at about 15.3, is characterized by a powder X-ray diffraction pattern that has a characteristic peak in degrees 2θ at about 17.8, is characterized by a powder X-ray diffraction pattern that has a characteristic peak in degrees 2θ at about 18.7, is characterized by a powder X-ray diffraction pattern that has a characteristic peak in degrees 2θ at about 22.6, and/or is characterized by a powder X-ray diffraction pattern that has a characteristic peak in degrees 2θ at about 26.8. In yet another embodiment, crystalline Form F is characterized by a powder X-ray diffraction pattern having at least one or more characteristic peaks in degrees 2θ at about 4.5, 7.0, 8.2, 9.0, 9.3, 12.5, 13.5, 15.3, 17.8, 18.7, 22.6, and 26.8. For example, a contemplated crystalline form has a powder X-ray diffraction pattern shown in FIG. 9. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, characterized by a powder X-ray diffraction pattern having a characteristic peak in degrees 2θ at about 8.5 (referred to herein as “Form H”), is disclosed herein.

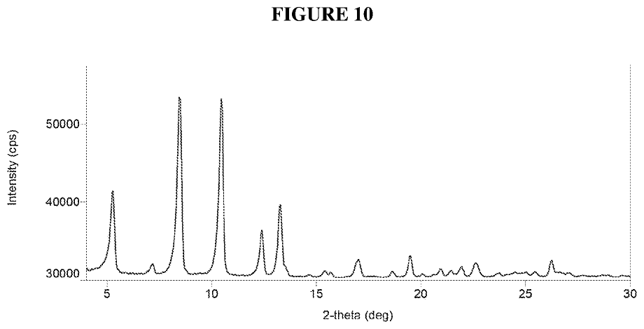

In one embodiment, the crystalline Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, is characterized by a powder X-ray diffraction pattern that has a characteristic peak in degrees 2θ at about 5.3, is characterized by a powder X-ray diffraction pattern that has a characteristic peak in degrees 2θ at about 7.2, is characterized by a powder X-ray diffraction pattern that has a characteristic peak in degrees 2θ at about 10.4, is characterized by a powder X-ray diffraction pattern that has a characteristic peak in degrees 2θ at about 12.4, is characterized by a powder X-ray diffraction pattern that has a characteristic peak in degrees 2θ at about 13.3, is characterized by a powder X-ray diffraction pattern that has a characteristic peak in degrees 2θ at about 17.0, is characterized by a powder X-ray diffraction pattern that has a characteristic peak in degrees 2θ at about 19.5, is characterized by a powder X-ray diffraction pattern that has a characteristic peak in degrees 2θ at about 22.6, is characterized by a powder X-ray diffraction pattern that has a characteristic peak in degrees 2θ at about 24.5, is characterized by a powder X-ray diffraction pattern that has a characteristic peak in degrees 2θ at about 26.2, and/or is characterized by a powder X-ray diffraction pattern that has a characteristic peak in degrees 2θ at about 26.7. In yet another embodiment, crystalline Form H is characterized by a powder X-ray diffraction pattern having at least one or more characteristic peaks in degrees 2θ at about 5.3, 7.2, 8.5, 10.4, 12.4, 13.3, 17.0, 19.5, 22.6, 24.5, 26.2, and 26.7. For example, a contemplated crystalline form has a powder X-ray diffraction pattern shown in FIG. 10. In one embodiment, the powder X-ray diffraction pattern of the crystalline form was obtained using Cu Kα radiation.

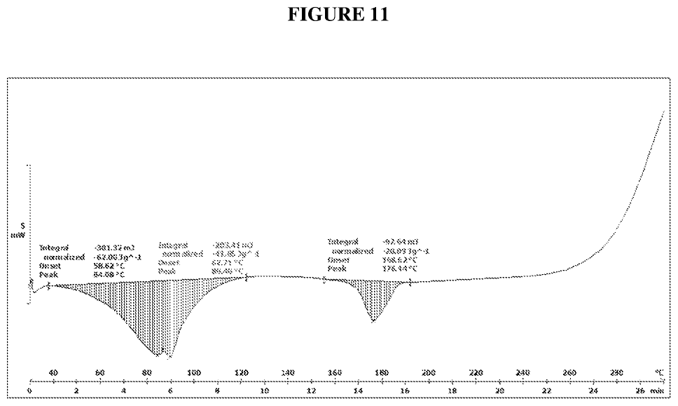

The contemplated crystalline Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, may be characterized by a differential scanning calorimetry (DSC) profile showing a characteristic endotherm with an onset of about 58° C. and a peak of about 84° C., a characteristic endotherm with an onset of about 63° C. and a peak of about 89° C., and a characteristic endotherm with an onset of about 169° C. and a peak of about 176° C. Form H, for example, may be characterized by the differential scanning calorimetry profile shown in FIG. 11.

The contemplated crystalline Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, may be characterized by a thermogravimetric analysis (TGA) profile showing a mass loss of about 6.3 wt. % up to about 130° C. In some embodiments, crystalline Form H may be characterized by a dynamic vapor sorption (DVS) profile showing a reversable total mass change of about 7.6 wt. % between about 2 to about 92% relative humidity (RH) at 25° C. In other embodiments, crystalline Form H may be characterized by optical microscopy showing a hair-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.4 (referred to herein as “Form I”), is disclosed herein.

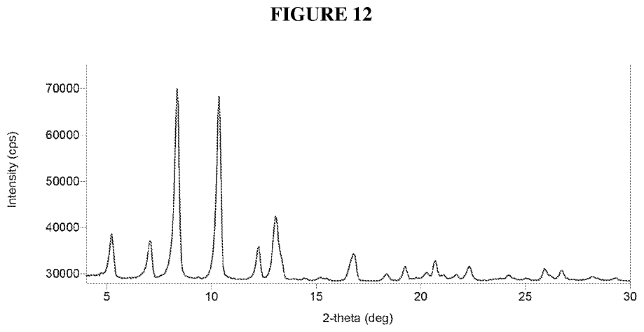

In one embodiment, the crystalline Form I of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 5.2, is characterized by a powder X-ray diffraction pattern that has a characteristic peak in degrees 2θ at about 7.0, is characterized by a powder X-ray diffraction pattern that has a characteristic peak in degrees 2θ at about 10.4, is characterized by a powder X-ray diffraction pattern that has a characteristic peak in degrees 2θ at about 12.2, is characterized by a powder X-ray diffraction pattern that has a characteristic peak in degrees 2θ at about 13.1, is characterized by a powder X-ray diffraction pattern that has a characteristic peak in degrees 2θ at about 16.8, is characterized by a powder X-ray diffraction pattern that has a characteristic peak in degrees 2θ at about 19.2, is characterized by a powder X-ray diffraction pattern that has a characteristic peak in degrees 2θ at about 20.7, is characterized by a powder X-ray diffraction pattern that has a characteristic peak in degrees 2θ at about 22.3, and/or is characterized by a powder X-ray diffraction pattern that has a characteristic peak in degrees 2θ at about 25.9. In yet another embodiment, crystalline Form I is characterized by a powder X-ray diffraction pattern having at least one or more characteristic peaks in degrees 2θ at about 5.2, 7.0, 8.4, 10.4, 12.2, 13.1, 16.8, 19.2, 20.7, 22.3, and 25.9. For example, a contemplated crystalline form has a powder X-ray diffraction pattern shown in FIG. 12. In one embodiment, the powder X-ray diffraction pattern of the crystalline form was obtained using Cu Kα radiation. In other embodiments, crystalline Form I may be characterized by optical microscopy showing a hair-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 4.1 (referred to herein as “Form J”), is disclosed herein.

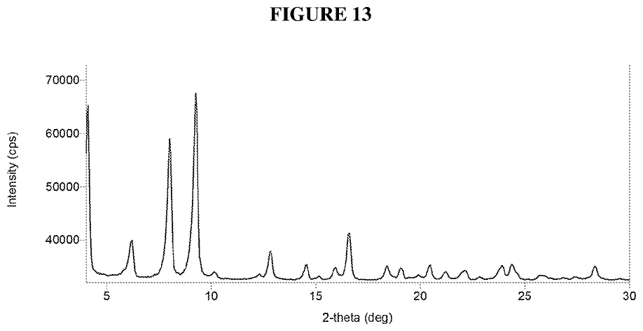

In one embodiment, the crystalline Form J of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 6.2, is characterized by a powder X-ray diffraction pattern that has a characteristic peak in degrees 2θ at about 8.0, is characterized by a powder X-ray diffraction pattern that has a characteristic peak in degrees 2θ at about 9.3, is characterized by a powder X-ray diffraction pattern that has a characteristic peak in degrees 2θ at about 12.8, is characterized by a powder X-ray diffraction pattern that has a characteristic peak in degrees 2θ at about 16.6, is characterized by a powder X-ray diffraction pattern that has a characteristic peak in degrees 2θ at about 18.4, is characterized by a powder X-ray diffraction pattern that has a characteristic peak in degrees 2θ at about 23.8, is characterized by a powder X-ray diffraction pattern that has a characteristic peak in degrees 2θ at about 24.4, and/or is characterized by a powder X-ray diffraction pattern that has a characteristic peak in degrees 2θ at about 28.3. In yet another embodiment, crystalline Form J is characterized by a powder X-ray diffraction pattern having at least one or more characteristic peaks in degrees 2θ at about 4.1, 6.2, 8.0, 9.3, 12.8, 16.6, 18.4, 23.8, 24.4, and 28.3. For example, a contemplated crystalline form has a powder X-ray diffraction pattern shown in FIG. 13. In one embodiment, the powder X-ray diffraction pattern of the crystalline form was obtained using Cu Kα radiation. In other embodiments, crystalline Form J may be characterized by optical microscopy showing a hair-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 9.6 (referred to herein as “Form K”), is disclosed herein.

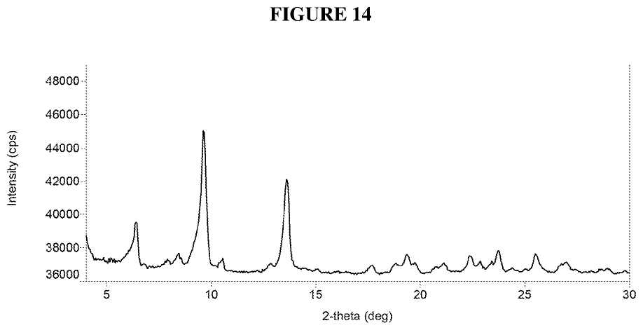

In one embodiment, the crystalline Form K of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 6.4, is characterized by a powder X-ray diffraction pattern that has a characteristic peak in degrees 2θ at about 10.5, is characterized by a powder X-ray diffraction pattern that has a characteristic peak in degrees 2θ at about 13.6, is characterized by a powder X-ray diffraction pattern that has a characteristic peak in degrees 2θ at about 19.4, is characterized by a powder X-ray diffraction pattern that has a characteristic peak in degrees 2θ at about 21.0, is characterized by a powder X-ray diffraction pattern that has a characteristic peak in degrees 2θ at about 22.4, is characterized by a powder X-ray diffraction pattern that has a characteristic peak in degrees 2θ at about 23.4, is characterized by a powder X-ray diffraction pattern that has a characteristic peak in degrees 2θ at about 23.7, is characterized by a powder X-ray diffraction pattern that has a characteristic peak in degrees 2θ at about 25.2, and/or is characterized by a powder X-ray diffraction pattern that has a characteristic peak in degrees 2θ at about 26.9. In yet another embodiment, crystalline Form K is characterized by a powder X-ray diffraction pattern having at least one or more characteristic peaks in degrees 2θ at about 6.4, 9.6, 10.5, 13.6, 19.4, 21.0, 22.4, 23.4, 23.7, 25.2, and 26.9. For example, a contemplated crystalline form has a powder X-ray diffraction pattern shown in FIG. 14. In one embodiment, the powder X-ray diffraction pattern of the crystalline form was obtained using Cu Kα radiation.

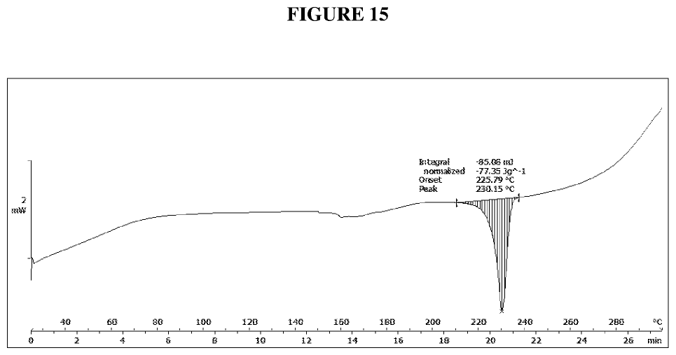

The contemplated crystalline Form K of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a differential scanning calorimetry (DSC) profile showing a characteristic endotherm with an onset of about 226° C. and a peak of about 230° C. Form K, for example, may be characterized by the differential scanning calorimetry profile shown in FIG. 15.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.3 (referred to herein as “Form L”), is disclosed herein.

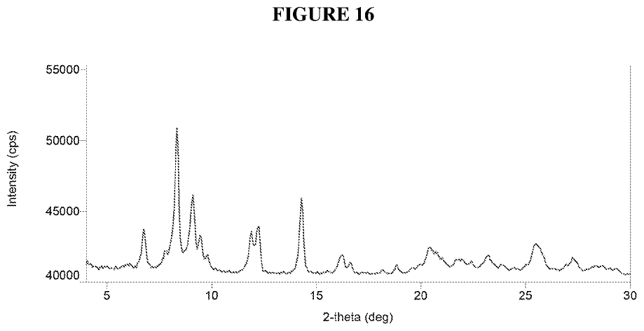

In one embodiment, the crystalline Form L of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 9.1, is characterized by a powder X-ray diffraction pattern that has a characteristic peak in degrees 2θ at about 9.5, is characterized by a powder X-ray diffraction pattern that has a characteristic peak in degrees 2θ at about 11.9, is characterized by a powder X-ray diffraction pattern that has a characteristic peak in degrees 2θ at about 12.2, is characterized by a powder X-ray diffraction pattern that has a characteristic peak in degrees 2θ at about 14.3, is characterized by a powder X-ray diffraction pattern that has a characteristic peak in degrees 2θ at about 16.2, is characterized by a powder X-ray diffraction pattern that has a characteristic peak in degrees 2θ at about 20.5, is characterized by a powder X-ray diffraction pattern that has a characteristic peak in degrees 2θ at about 21.9, is characterized by a powder X-ray diffraction pattern that has a characteristic peak in degrees 2θ at about 23.1, and/or is characterized by a powder X-ray diffraction pattern that has a characteristic peak in degrees 2θ at about 25.5. In yet another embodiment, crystalline Form L is characterized by a powder X-ray diffraction pattern having at least one or more characteristic peaks in degrees 2θ at about 6.7, 8.3, 9.1, 9.5, 11.9, 12.2, 14.3, 16.2, 20.5, 21.9, 23.1, and 25.5. For example, a contemplated crystalline form has a powder X-ray diffraction pattern shown in FIG. 16. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 9.6 (referred to herein as “Form M”), is disclosed herein.

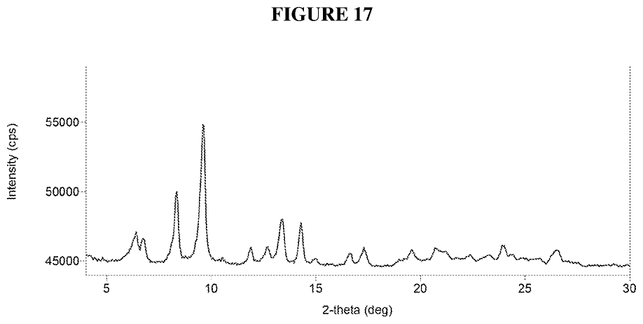

In one embodiment, the crystalline Form M of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 6.8, is characterized by a powder X-ray diffraction pattern that has a characteristic peak in degrees 2θ at about 8.3, is characterized by a powder X-ray diffraction pattern that has a characteristic peak in degrees 2θ at about 12.7, is characterized by a powder X-ray diffraction pattern that has a characteristic peak in degrees 2θ at about 13.4, is characterized by a powder X-ray diffraction pattern that has a characteristic peak in degrees 2θ at about 14.3, is characterized by a powder X-ray diffraction pattern that has a characteristic peak in degrees 2θ at about 17.3, is characterized by a powder X-ray diffraction pattern that has a characteristic peak in degrees 2θ at about 19.6, is characterized by a powder X-ray diffraction pattern that has a characteristic peak in degrees 2θ at about 21.0, is characterized by a powder X-ray diffraction pattern that has a characteristic peak in degrees 2θ at about 23.9, and/or is characterized by a powder X-ray diffraction pattern that has a characteristic peak in degrees 2θ at about 26.5. In yet another embodiment, crystalline Form M is characterized by a powder X-ray diffraction pattern having at least one or more characteristic peaks in degrees 2θ at about 6.4, 6.8, 8.3, 9.6, 12.7, 13.4, 14.3, 17.3, 19.6, 21.0, 23.9, and 26.5. For example, a contemplated crystalline form has a powder X-ray diffraction pattern shown in FIG. 17. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.4 (referred to herein as “Form N”), is disclosed herein.

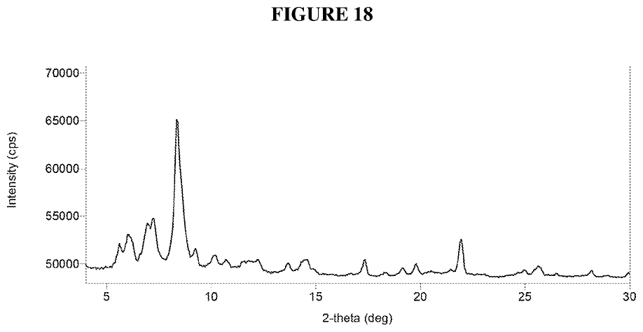

In one embodiment, the crystalline Form N of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 5.6, is characterized by a powder X-ray diffraction pattern that has a characteristic peak in degrees 2θ at about 6.1, is characterized by a powder X-ray diffraction pattern that has a characteristic peak in degrees 2θ at about 6.9, is characterized by a powder X-ray diffraction pattern that has a characteristic peak in degrees 2θ at about 7.2, is characterized by a powder X-ray diffraction pattern that has a characteristic peak in degrees 2θ at about 11.8, is characterized by a powder X-ray diffraction pattern that has a characteristic peak in degrees 2θ at about 13.7, is characterized by a powder X-ray diffraction pattern that has a characteristic peak in degrees 2θ at about 14.5, is characterized by a powder X-ray diffraction pattern that has a characteristic peak in degrees 2θ at about 17.3, is characterized by a powder X-ray diffraction pattern that has a characteristic peak in degrees 2θ at about 19.8, is characterized by a powder X-ray diffraction pattern that has a characteristic peak in degrees 2θ at about 21.9, is characterized by a powder X-ray diffraction pattern that has a characteristic peak in degrees 2θ at about 24.9, and/or is characterized by a powder X-ray diffraction pattern that has a characteristic peak in degrees 2θ at about 25.7. In yet another embodiment, crystalline Form N is characterized by a powder X-ray diffraction pattern having at least one or more characteristic peaks in degrees 2θ at about 5.6, 6.1, 6.9, 7.2, 8.4, 11.8, 13.7, 14.5, 17.3, 19.8, 21.9, 24.9, and 25.7. For example, a contemplated crystalline form has a powder X-ray diffraction pattern shown in FIG. 18. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 14.1 (referred to herein as “Form O”), is disclosed herein.

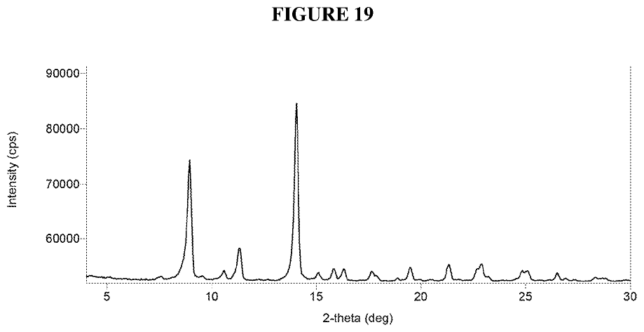

In one embodiment, the crystalline Form O of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 9.0, is characterized by a powder X-ray diffraction pattern that has a characteristic peak in degrees 2θ at about 10.6, is characterized by a powder X-ray diffraction pattern that has a characteristic peak in degrees 2θ at about 11.3, is characterized by a powder X-ray diffraction pattern that has a characteristic peak in degrees 2θ at about 15.8, is characterized by a powder X-ray diffraction pattern that has a characteristic peak in degrees 2θ at about 16.3, is characterized by a powder X-ray diffraction pattern that has a characteristic peak in degrees 2θ at about 17.6, is characterized by a powder X-ray diffraction pattern that has a characteristic peak in degrees 2θ at about 19.5, is characterized by a powder X-ray diffraction pattern that has a characteristic peak in degrees 2θ at about 21.3, is characterized by a powder X-ray diffraction pattern that has a characteristic peak in degrees 2θ at about 22.7, is characterized by a powder X-ray diffraction pattern that has a characteristic peak in degrees 2θ at about 22.9, is characterized by a powder X-ray diffraction pattern that has a characteristic peak in degrees 2θ at about 25.1, and/or is characterized by a powder X-ray diffraction pattern that has a characteristic peak in degrees 2θ at about 28.7. In yet another embodiment, crystalline Form O is characterized by a powder X-ray diffraction pattern having at least one or more characteristic peaks in degrees 2θ at about 9.0, 10.6, 11.3, 14.1, 15.8, 16.3, 17.6, 19.5, 21.3, 22.7, 22.9, 25.1, and 28.7. For example, a contemplated crystalline form has a powder X-ray diffraction pattern shown in FIG. 19. In one embodiment, the powder X-ray diffraction pattern of the crystalline form was obtained using Cu Kα radiation.

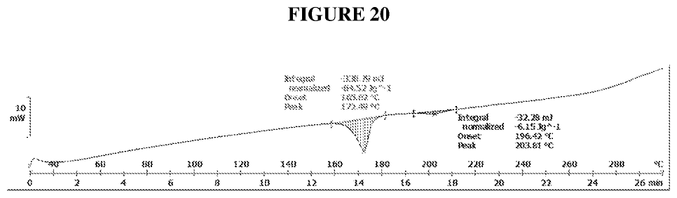

The contemplated crystalline Form O of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a differential scanning calorimetry (DSC) profile showing a characteristic endotherm with an onset of about 166° C. and a peak of about 172° C., and a characteristic endotherm with an onset of about 196° C. and a peak of about 204° C. Form O, for example, may be characterized by the differential scanning calorimetry profile shown in FIG. 20.

maintained crystallinity and solid form purity through a preliminary wet-milling study (ball mill, 30 s milled with 1 vol. water). In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 7.7 (referred to herein as “Form Q”), is disclosed herein.

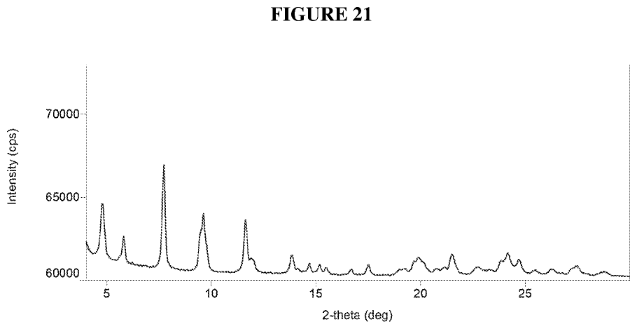

In one embodiment, the crystalline Form Q of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 4.8, is characterized by a powder X-ray diffraction pattern that has a characteristic peak in degrees 2θ at about 5.8, is characterized by a powder X-ray diffraction pattern that has a characteristic peak in degrees 2θ at about 9.6, is characterized by a powder X-ray diffraction pattern that has a characteristic peak in degrees 2θ at about 11.6, is characterized by a powder X-ray diffraction pattern that has a characteristic peak in degrees 2θ at about 13.8, is characterized by a powder X-ray diffraction pattern that has a characteristic peak in degrees 2θ at about 14.7, is characterized by a powder X-ray diffraction pattern that has a characteristic peak in degrees 2θ at about 19.9, is characterized by a powder X-ray diffraction pattern that has a characteristic peak in degrees 2θ at about 21.5, is characterized by a powder X-ray diffraction pattern that has a characteristic peak in degrees 2θ at about 24.1, is characterized by a powder X-ray diffraction pattern that has a characteristic peak in degrees 2θ at about 24.7, and/or is characterized by a powder X-ray diffraction pattern that has a characteristic peak in degrees 2θ at about 27.4. In yet another embodiment, crystalline Form Q is characterized by a powder X-ray diffraction pattern having at least one or more characteristic peaks in degrees 2θ at about 4.8, 5.8, 7.7, 9.6, 11.6, 13.8, 14.7, 19.9, 21.5, 24.1, 24.7, and 27.4. For example, a contemplated crystalline form has a powder X-ray diffraction pattern shown in FIG. 21. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.5 (referred to herein as “Form R”), is disclosed herein.

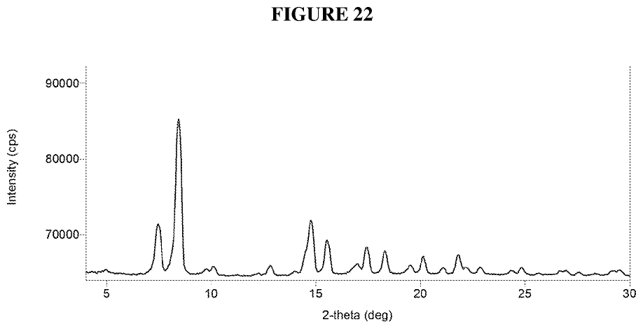

In one embodiment, the crystalline Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 7.5, is characterized by a powder X-ray diffraction pattern that has a characteristic peak in degrees 2θ at about 10.1, is characterized by a powder X-ray diffraction pattern that has a characteristic peak in degrees 2θ at about 12.8, is characterized by a powder X-ray diffraction pattern that has a characteristic peak in degrees 2θ at about 14.8, is characterized by a powder X-ray diffraction pattern that has a characteristic peak in degrees 2θ at about 15.6, is characterized by a powder X-ray diffraction pattern that has a characteristic peak in degrees 2θ at about 16.9, is characterized by a powder X-ray diffraction pattern that has a characteristic peak in degrees 2θ at about 17.4, is characterized by a powder X-ray diffraction pattern that has a characteristic peak in degrees 2θ at about 18.3, is characterized by a powder X-ray diffraction pattern that has a characteristic peak in degrees 2θ at about 19.5, is characterized by a powder X-ray diffraction pattern that has a characteristic peak in degrees 2θ at about 20.0, and/or is characterized by a powder X-ray diffraction pattern that has a characteristic peak in degrees 2θ at about 21.8. In yet another embodiment, crystalline Form R is characterized by a powder X-ray diffraction pattern having at least one or more characteristic peaks in degrees 2θ at about 7.5, 8.5, 10.1, 12.8, 14.8, 15.6, 16.9, 17.4, 18.3, 19.5, 20.0, and 21.8. For example, a contemplated crystalline form has a powder X-ray diffraction pattern shown in FIG. 22. In one embodiment, the powder X-ray diffraction pattern of the crystalline form was obtained using Cu Kα radiation.

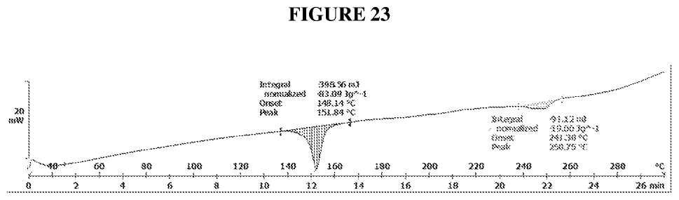

The contemplated crystalline Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a differential scanning calorimetry (DSC) profile showing a characteristic endotherm with an onset of about 148° C. and a peak of about 152° C., and a characteristic endotherm with an onset of about 241° C. and a peak of about 251° C. Form R, for example, may be characterized by the differential scanning calorimetry profile shown in FIG. 23.

The contemplated crystalline Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a thermogravimetric analysis (TGA) profile showing a first step mass loss of about 0.87 wt. % up to about 150° C., a second step mass loss of about 3.1 wt. % between about 150° C. to about 200° C., and a third step mass loss of about 11.7 wt. % between about 200° C. to about 240° C.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 5.0 (referred to herein as “Form S”), is disclosed herein.

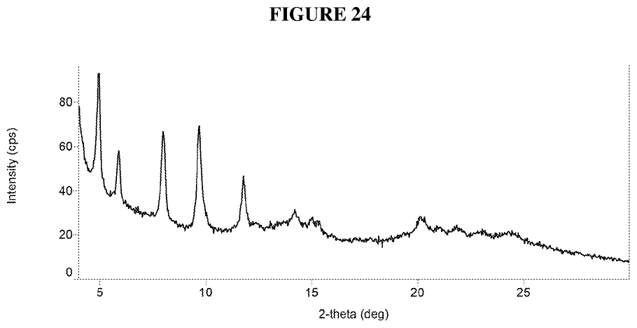

In one embodiment, the crystalline Form S of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 5.9, is characterized by a powder X-ray diffraction pattern that has a characteristic peak in degrees 2θ at about 8.0, is characterized by a powder X-ray diffraction pattern that has a characteristic peak in degrees 2θ at about 9.7, and/or is characterized by a powder X-ray diffraction pattern that has a characteristic peak in degrees 2θ at about 11.8. In yet another embodiment, crystalline Form S is characterized by a powder X-ray diffraction pattern having at least one or more characteristic peaks in degrees 2θ at about 5.0, 5.9, 8.0, 9.7, and 11.8. For example, a contemplated crystalline form has a powder X-ray diffraction pattern shown in FIG. 24. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.7 (referred to herein as “Form T”), is disclosed herein.

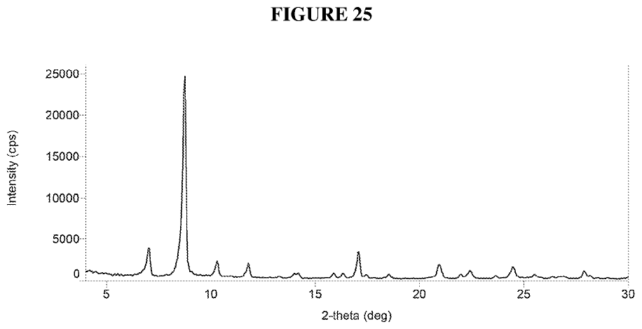

In one embodiment, the crystalline Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 7.0, is characterized by a powder X-ray diffraction pattern that has a characteristic peak in degrees 2θ at about 10.3, is characterized by a powder X-ray diffraction pattern that has a characteristic peak in degrees 2θ at about 11.8, is characterized by a powder X-ray diffraction pattern that has a characteristic peak in degrees 2θ at about 14.1, is characterized by a powder X-ray diffraction pattern that has a characteristic peak in degrees 2θ at about 17.0, is characterized by a powder X-ray diffraction pattern that has a characteristic peak in degrees 2θ at about 20.9, is characterized by a powder X-ray diffraction pattern that has a characteristic peak in degrees 2θ at about 22.4, is characterized by a powder X-ray diffraction pattern that has a characteristic peak in degrees 2θ at about 24.5, is characterized by a powder X-ray diffraction pattern that has a characteristic peak in degrees 2θ at about 25.5, and/or is characterized by a powder X-ray diffraction pattern that has a characteristic peak in degrees 2θ at about 27.9. In yet another embodiment, crystalline Form T is characterized by a powder X-ray diffraction pattern having at least one or more characteristic peaks in degrees 2θ at about 7.0, 8.7, 10.3, 11.8, 14.1, 17.0, 20.9, 22.4, 24.5, 25.5, and 27.9. For example, a contemplated crystalline form has a powder X-ray diffraction pattern shown in FIG. 25. In one embodiment, the powder X-ray diffraction pattern of the crystalline form was obtained using Cu Kα radiation.

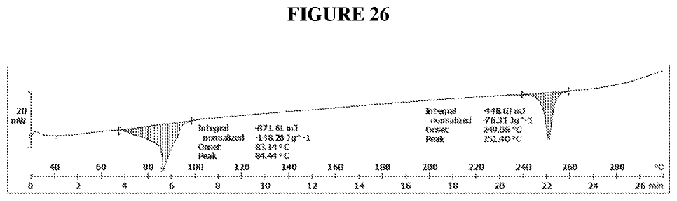

The contemplated crystalline Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a differential scanning calorimetry (DSC) profile showing a characteristic endotherm with an onset of about 83° C. and a peak of about 84° C., and a characteristic endotherm with an onset of about 249° C. and a peak of about 251° C. Form T, for example, may be characterized by the differential scanning calorimetry profile shown in FIG. 26.

The contemplated crystalline Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, may be characterized by a thermogravimetric analysis (TGA) profile showing a first step mass loss of about 13.8 wt. % up to about 85° C. In other embodiments, crystalline Form T may be characterized by optical microscopy showing a needle-like and/or a rod-like morphology.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 6.2 (referred to herein as “Form U”), is disclosed herein.

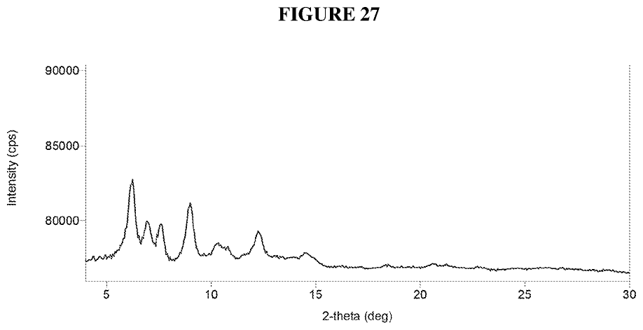

In one embodiment, the crystalline Form U of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 6.9, is characterized by a powder X-ray diffraction pattern that has a characteristic peak in degrees 2θ at about 7.6, is characterized by a powder X-ray diffraction pattern that has a characteristic peak in degrees 2θ at about 9.0, is characterized by a powder X-ray diffraction pattern that has a characteristic peak in degrees 2θ at about 10.5, is characterized by a powder X-ray diffraction pattern that has a characteristic peak in degrees 2θ at about 12.3, and/or is characterized by a powder X-ray diffraction pattern that has a characteristic peak in degrees 2θ at about 14.5. In yet another embodiment, crystalline Form U is characterized by a powder X-ray diffraction pattern having at least one or more characteristic peaks in degrees 2θ at about 6.2, 6.9, 7.6, 9.0, 10.5, 12.3, and 14.5. For example, a contemplated crystalline form has a powder X-ray diffraction pattern shown in FIG. 27. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg] [1,4]oxazonine, free base, characterized by a powder X-ray diffraction pattern having a characteristic peak in degrees 2θ at about 18.3 (referred to herein as “Form V”), is disclosed herein.

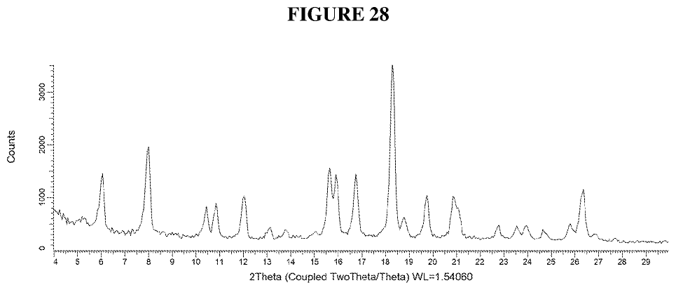

In one embodiment, the crystalline Form V of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 6.0, is characterized by a powder X-ray diffraction pattern that has a characteristic peak in degrees 2θ at about 8.0, is characterized by a powder X-ray diffraction pattern that has a characteristic peak in degrees 2θ at about 10.8, is characterized by a powder X-ray diffraction pattern that has a characteristic peak in degrees 2θ at about 12.0, is characterized by a powder X-ray diffraction pattern that has a characteristic peak in degrees 2θ at about 15.6, is characterized by a powder X-ray diffraction pattern that has a characteristic peak in degrees 2θ at about 15.9, is characterized by a powder X-ray diffraction pattern that has a characteristic peak in degrees 2θ at about 16.8, is characterized by a powder X-ray diffraction pattern that has a characteristic peak in degrees 2θ at about 19.7, is characterized by a powder X-ray diffraction pattern that has a characteristic peak in degrees 2θ at about 20.9, is characterized by a powder X-ray diffraction pattern that has a characteristic peak in degrees 2θ at about 21.0, and/or is characterized by a powder X-ray diffraction pattern that has a characteristic peak in degrees 2θ at about 26.3. In yet another embodiment, crystalline Form V is characterized by a powder X-ray diffraction pattern having at least one or more characteristic peaks in degrees 2θ at about 6.0, 8.0, 10.8, 12.0, 15.6, 15.9, 16.8, 18.3, 19.7, 20.9, 21.0, and 26.3. For example, a contemplated crystalline form has a powder X-ray diffraction pattern shown in FIG. 28. In one embodiment, the powder X-ray diffraction pattern of the crystalline form was obtained using Cu Kα radiation.

In another embodiment, a different crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, characterized by a powder X-ray diffraction pattern having a characteristic peak in degrees 2θ at about 8.6 (referred to herein as “Form W”), is disclosed herein.

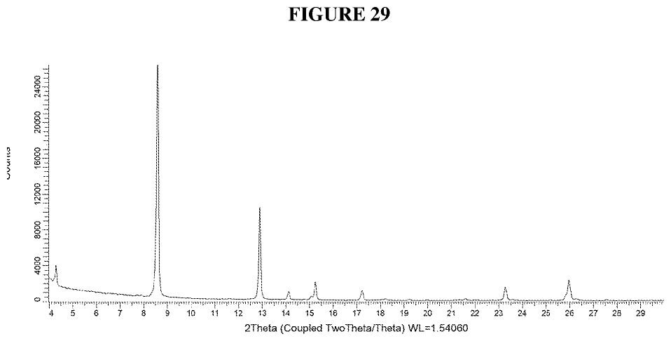

In one embodiment, the crystalline Form W of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is characterized by a powder X-ray diffraction pattern that has a characteristic peak in degrees 2θ at about 4.3, is characterized by a powder X-ray diffraction pattern that has a characteristic peak in degrees 2θ at about 12.9, is characterized by a powder X-ray diffraction pattern that has a characteristic peak in degrees 2θ at about 15.2, is characterized by a powder X-ray diffraction pattern that has a characteristic peak in degrees 2θ at about 23.3, and/or is characterized by a powder X-ray diffraction pattern that has a characteristic peak in degrees 2θ at about 26.0. In yet another embodiment, crystalline Form W is characterized by a powder X-ray diffraction pattern having at least one or more characteristic peaks in degrees 2θ at about 4.3, 8.6, 12.9, 15.2, 23.3, and 26.0. For example, a contemplated crystalline form has a powder X-ray diffraction pattern shown in FIG. 29. In one embodiment, the powder X-ray diffraction pattern of the crystalline form was obtained using Cu Kα radiation.

In another embodiment, a substantially amorphous form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is disclosed herein.

In a further embodiment, a pharmaceutical composition comprising a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base and a pharmaceutically acceptable excipient is disclosed herein. For example, a pharmaceutical composition comprising the crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, and a pharmaceutically acceptable excipient is disclosed herein. For example, a pharmaceutical composition formed from the crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is disclosed herein. In some embodiments, a disclosed pharmaceutical composition may be a formulation for oral administration.

In yet another embodiment, a pharmaceutical composition comprising a disclosed amorphous form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base and a pharmaceutically acceptable excipient is disclosed herein.

In an embodiment, a drug substance comprising at least a detectable amount of a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is disclosed herein. In another embodiment, a drug substance comprising a substantially pure crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is disclosed herein. For example, a drug substance comprising a substantially pure crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is disclosed herein.

Also disclosed herein is a pharmaceutically acceptable salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In some embodiments the salt may be selected from the group consisting of, for example, a benzenesulfonic acid salt, a citric acid salt, a fumaric acid salt, a maleic acid salt, a L-malic acid salt, a methanesulfonic acid salt, a phosphoric acid salt, a pyruvic acid salt, a sulfuric acid salt, a L-tartaric acid salt, a toluenesulfonic acid salt, and hydrates and solvates thereof. In some embodiments, the salt may be, for example, a benzenesulfonic acid salt. In some embodiments, the salt may be, for example, a citric acid salt. In some embodiments, the salt may be, for example, a fumaric acid salt. In some embodiments, the salt may be, for example, a hydrochloric acid salt. In some embodiments, the salt may be, for example, a L-malic acid salt. In some embodiments, the salt may be, for example, a methanesulfonic acid salt. In some embodiments, the salt may be, for example, a phosphoric acid salt. In some embodiments, the salt may be, for example, a pyruvic acid salt. In some embodiments, the salt may be, for example, a sulfuric acid salt. In some embodiments, the salt may be, for example, a L-tartaric acid salt. In some embodiments, salt may be, for example, a toluenesulfonic acid salt.

In a further embodiment, a pharmaceutical composition comprising a disclosed salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient is disclosed herein. For example, a pharmaceutical composition formed from a disclosed salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein. In some embodiments, a disclosed pharmaceutical composition is a formulation for oral administration.

In an embodiment, a drug substance comprising at least a detectable amount of a disclosed salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein. In another embodiment, a drug substance comprising a substantially pure salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein.

Further disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In some embodiments, the crystalline salt form may be selected from the group consisting of, for example, a benzenesulfonic acid salt, a citric acid salt, a fumaric acid salt, a hydrochloride salt, a maleic acid salt, a L-malic acid salt, a methanesulfonic acid salt, a phosphoric acid salt, a pyruvic acid salt, a sulfuric acid salt, a L-tartaric acid salt, and a toluenesulfonic acid salt, and crystalline hydrates and solvates thereof. In some embodiments, the crystalline salt form may be, for example, a benzenesulfonic acid salt. In some embodiments, the crystalline salt form may be, for example, a citric acid salt. In some embodiments, the crystalline salt form may be, for example, a fumaric acid salt. In some embodiments, the crystalline salt form may be, for example, a hydrochloric acid salt. In some embodiments, the crystalline salt form may be, for example, a L-malic acid salt. In some embodiments, the crystalline salt form may be, for example, a methanesulfonic acid salt. In some embodiments, the crystalline salt form may be, for example, a phosphoric acid salt. In some embodiments, the crystalline salt form may be, for example, a pyruvic acid salt. In some embodiments, the crystalline salt form may be, for example, a sulfuric acid salt. In some embodiments, the crystalline salt form may be, for example, a L-tartaric acid salt. In some embodiments, the crystalline salt form may be, for example, a toluenesulfonic acid salt.

For example, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, characterized by a powder X-ray diffraction pattern having a characteristic peak in degrees 2θ at about 6.5 (referred to herein as “Form 1-A”).

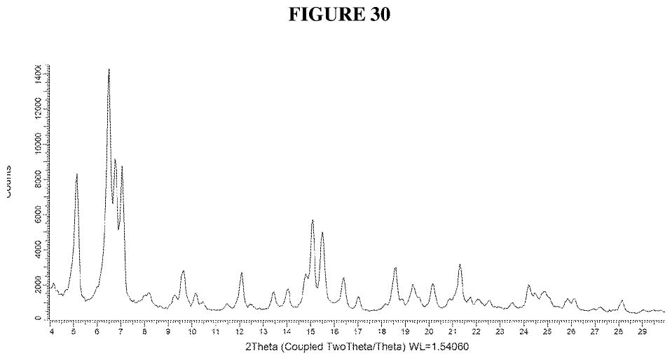

In one embodiment, the crystalline salt Form 1-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, is characterized by a powder X-ray diffraction pattern that has a characteristic peak in degrees 2θ at about 5.1, is characterized by a powder X-ray diffraction pattern that has a characteristic peak in degrees 2θ at about 6.8, is characterized by a powder X-ray diffraction pattern that has a characteristic peak in degrees 2θ at about 7.0, is characterized by a powder X-ray diffraction pattern that has a characteristic peak in degrees 2θ at about 9.6, is characterized by a powder X-ray diffraction pattern that has a characteristic peak in degrees 2θ at about 12.1, is characterized by a powder X-ray diffraction pattern that has a characteristic peak in degrees 2θ at about 14.8, is characterized by a powder X-ray diffraction pattern that has a characteristic peak in degrees 2θ at about 15.1, is characterized by a powder X-ray diffraction pattern that has a characteristic peak in degrees 2θ at about 15.5, is characterized by a powder X-ray diffraction pattern that has a characteristic peak in degrees 2θ at about 16.4, is characterized by a powder X-ray diffraction pattern that has a characteristic peak in degrees 2θ at about 18.6, and/or is characterized by a powder X-ray diffraction pattern that has a characteristic peak in degrees 2θ at about 21.3. In yet another embodiment, crystalline salt Form 1-A is characterized by a powder X-ray diffraction pattern having at least one or more characteristic peaks in degrees 2θ at about 5.1, 6.5, 6.8, 7.0, 9.6, 12.1, 14.8, 15.1, 15.5, 16.4, 18.6, and 21.3. For example, a contemplated crystalline salt form has a powder X-ray diffraction pattern shown in FIG. 30. In one embodiment, the powder X-ray diffraction pattern of the crystalline salt form was obtained using Cu Kα radiation.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, characterized by a powder X-ray diffraction pattern having a characteristic peak in degrees 2θ at about 6.7 (referred to herein as “Form 1-B”), is disclosed herein.

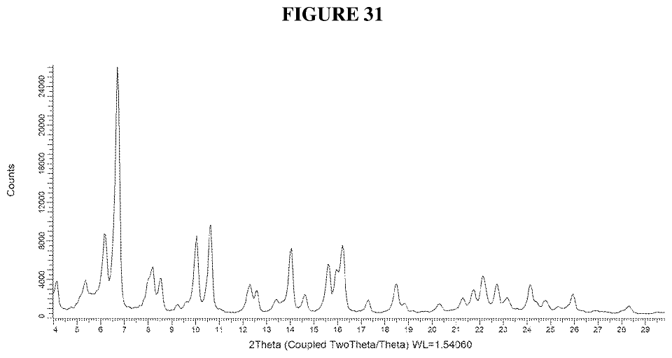

In one embodiment, the crystalline salt Form 1-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, is characterized by a powder X-ray diffraction pattern that has a characteristic peak in degrees 2θ at about 5.3, is characterized by a powder X-ray diffraction pattern that has a characteristic peak in degrees 2θ at about 6.2, is characterized by a powder X-ray diffraction pattern that has a characteristic peak in degrees 2θ at about 8.2, is characterized by a powder X-ray diffraction pattern that has a characteristic peak in degrees 2θ at about 10.0, is characterized by a powder X-ray diffraction pattern that has a characteristic peak in degrees 2θ at about 10.6, is characterized by a powder X-ray diffraction pattern that has a characteristic peak in degrees 2θ at about 12.3, is characterized by a powder X-ray diffraction pattern that has a characteristic peak in degrees 2θ at about 14.0, is characterized by a powder X-ray diffraction pattern that has a characteristic peak in degrees 2θ at about 15.6, is characterized by a powder X-ray diffraction pattern that has a characteristic peak in degrees 2θ at about 16.0, is characterized by a powder X-ray diffraction pattern that has a characteristic peak in degrees 2θ at about 16.2, and/or is characterized by a powder X-ray diffraction pattern that has a characteristic peak in degrees 2θ at about 22.1. In yet another embodiment, crystalline salt Form 1-B is characterized by a powder X-ray diffraction pattern having at least one or more characteristic peaks in degrees 2θ at about 5.3, 6.2, 6.7, 8,2, 10.0, 10.6, 12.3, 14.0, 15.6, 16.0, 16.2, and 22.1. For example, a contemplated crystalline salt form has a powder X-ray diffraction pattern shown in FIG. 31. In one embodiment, the powder X-ray diffraction pattern of the crystalline form was obtained using Cu Kα radiation.

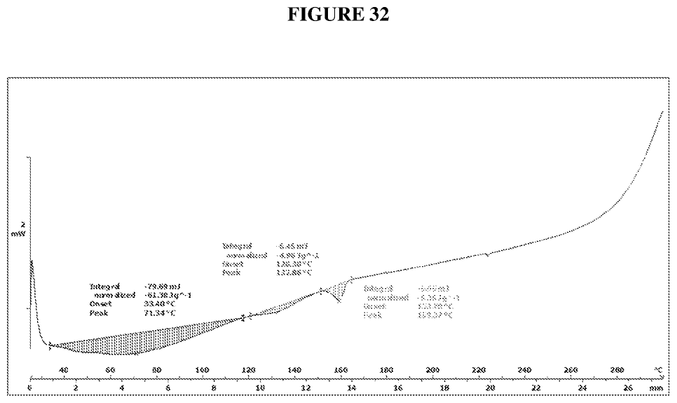

The contemplated crystalline Form 1-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, may be characterized by a differential scanning calorimetry (DSC) profile showing a characteristic endotherm with an onset of about 33° C. and a peak of about 71° C., a characteristic endotherm with an onset of about 120° C. and a peak of about 133° C., and a characteristic endotherm with an onset of about 154° C. and a peak of about 159° C. Form 1-B, for example, may be characterized by the differential scanning calorimetry profile shown in FIG. 32.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, characterized by a powder X-ray diffraction pattern having a characteristic peak in degrees 2θ at about 7.0 (referred to herein as “Form 2-A”).

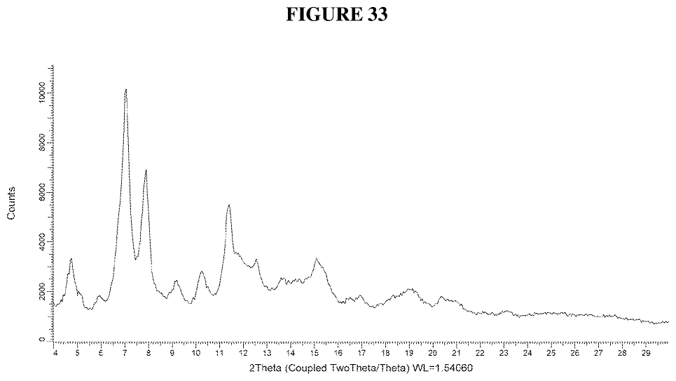

In one embodiment, the crystalline salt Form 2-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is characterized by a powder X-ray diffraction pattern that has a characteristic peak in degrees 2θ at about 4.7, is characterized by a powder X-ray diffraction pattern that has a characteristic peak in degrees 2θ at about 7.9, is characterized by a powder X-ray diffraction pattern that has a characteristic peak in degrees 2θ at about 9.1, is characterized by a powder X-ray diffraction pattern that has a characteristic peak in degrees 2θ at about 10.2, is characterized by a powder X-ray diffraction pattern that has a characteristic peak in degrees 2θ at about 11.4, is characterized by a powder X-ray diffraction pattern that has a characteristic peak in degrees 2θ at about 12.5, is characterized by a powder X-ray diffraction pattern that has a characteristic peak in degrees 2θ at about 13.7, is characterized by a powder X-ray diffraction pattern that has a characteristic peak in degrees 2θ at about 14.1, is characterized by a powder X-ray diffraction pattern that has a characteristic peak in degrees 2θ at about 15.1, is characterized by a powder X-ray diffraction pattern that has a characteristic peak in degrees 2θ at about 18.3, and/or is characterized by a powder X-ray diffraction pattern that has a characteristic peak in degrees 2θ at about 19.0. In yet another embodiment, crystalline salt Form 2-A is characterized by a powder X-ray diffraction pattern having at least one or more characteristic peaks in degrees 2θ at about 4.7, 7.0, 7.9, 9.1, 10.2, 11.4, 12.5, 13.7, 14.1, 15.1, 18.3, and 19.0. For example, a contemplated crystalline salt form has a powder X-ray diffraction pattern shown in FIG. 33. In one embodiment, the powder X-ray diffraction pattern of the crystalline form was obtained using Cu Kα radiation.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, characterized by a powder X-ray diffraction pattern having a characteristic peak in degrees 2θ at about 15.2 (referred to herein as “Form 2-B”), is disclosed herein.

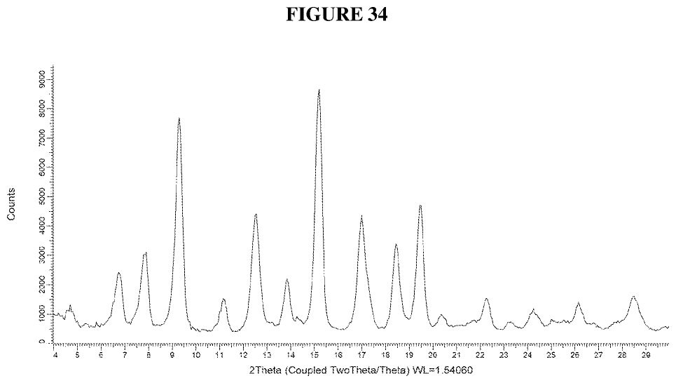

In one embodiment, the crystalline salt Form 2-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is characterized by a powder X-ray diffraction pattern that has a characteristic peak in degrees 2θ at about 6.7, is characterized by a powder X-ray diffraction pattern that has a characteristic peak in degrees 2θ at about 7.9, is characterized by a powder X-ray diffraction pattern that has a characteristic peak in degrees 2θ at about 9.3, is characterized by a powder X-ray diffraction pattern that has a characteristic peak in degrees 2θ at about 11.2, is characterized by a powder X-ray diffraction pattern that has a characteristic peak in degrees 2θ at about 12.5, is characterized by a powder X-ray diffraction pattern that has a characteristic peak in degrees 2θ at about 13.8, is characterized by a powder X-ray diffraction pattern that has a characteristic peak in degrees 2θ at about 17.0, is characterized by a powder X-ray diffraction pattern that has a characteristic peak in degrees 2θ at about 18.4, is characterized by a powder X-ray diffraction pattern that has a characteristic peak in degrees 2θ at about 19.5, is characterized by a powder X-ray diffraction pattern that has a characteristic peak in degrees 2θ at about 22.3, and/or is characterized by a powder X-ray diffraction pattern that has a characteristic peak in degrees 2θ at about 28.5. In yet another embodiment, crystalline salt Form 2-B is characterized by a powder X-ray diffraction pattern having at least one or more characteristic peaks in degrees 2θ at about 6.7, 7.9, 9.3, 11.2, 12.5, 13.8, 15.2, 17.0, 18.4, 19.5, 22.3, and 28.5. For example, a contemplated crystalline salt form has a powder X-ray diffraction pattern shown in FIG. 34. In one embodiment, the powder X-ray diffraction pattern of the crystalline form was obtained using Cu Kα radiation.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, characterized by a powder X-ray diffraction pattern having a characteristic peak in degrees 2θ at about 15.0 (referred to herein as “Form 2-C”), is disclosed herein.

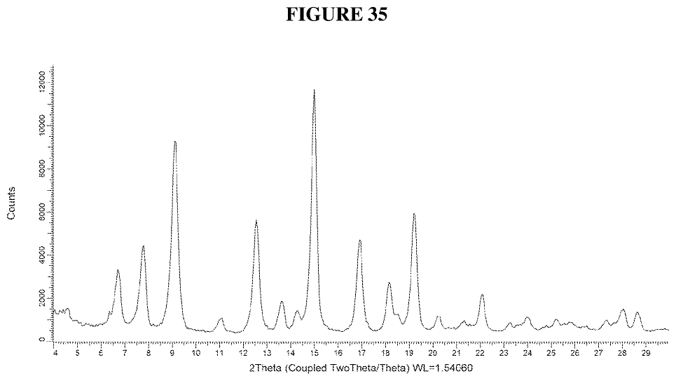

In one embodiment, the crystalline salt Form 2-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is characterized by a powder X-ray diffraction pattern that has a characteristic peak in degrees 2θ at about 6.7, is characterized by a powder X-ray diffraction pattern that has a characteristic peak in degrees 2θ at about 7.9, is characterized by a powder X-ray diffraction pattern that has a characteristic peak in degrees 2θ at about 9.1, is characterized by a powder X-ray diffraction pattern that has a characteristic peak in degrees 2θ at about 12.5, is characterized by a powder X-ray diffraction pattern that has a characteristic peak in degrees 2θ at about 13.6, is characterized by a powder X-ray diffraction pattern that has a characteristic peak in degrees 2θ at about 14.3, is characterized by a powder X-ray diffraction pattern that has a characteristic peak in degrees 2θ at about 16.9, is characterized by a powder X-ray diffraction pattern that has a characteristic peak in degrees 2θ at about 18.2, is characterized by a powder X-ray diffraction pattern that has a characteristic peak in degrees 2θ at about 19.2, is characterized by a powder X-ray diffraction pattern that has a characteristic peak in degrees 2θ at about 22.1, and/or is characterized by a powder X-ray diffraction pattern that has a characteristic peak in degrees 2θ at about 29.0. In yet another embodiment, crystalline salt Form 2-B is characterized by a powder X-ray diffraction pattern having at least one or more characteristic peaks in degrees 2θ at about 6.7, 7.9, 9.1, 12.5, 13.6, 14.3, 15.0, 16.9, 18.2, 19.2, 22.1, and 29.0. For example, a contemplated crystalline salt form has a powder X-ray diffraction pattern shown in FIG. 35. In one embodiment, the powder X-ray diffraction pattern of the crystalline form was obtained using Cu Kα radiation.

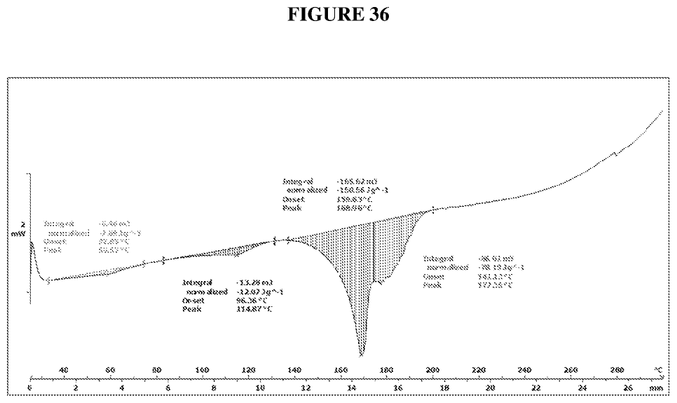

The contemplated crystalline Form 2-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, may be characterized by a differential scanning calorimetry (DSC) profile showing a characteristic endotherm with an onset of about 33° C. and a peak of about 60° C., a characteristic endotherm with an onset of about 96° C. and a peak of about 116° C., a characteristic endotherm with an onset of about 160° C. and a peak of about 169° C., and a characteristic endotherm with an onset of about 141° C. and a peak of about 177° C. Form 2-C, for example, may be characterized by the differential scanning calorimetry profile shown in FIG. 36.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, fumaric acid salt, characterized by a powder X-ray diffraction pattern having a characteristic peak in degrees 2θ at about 7.1 (referred to herein as “Form 3-A”).

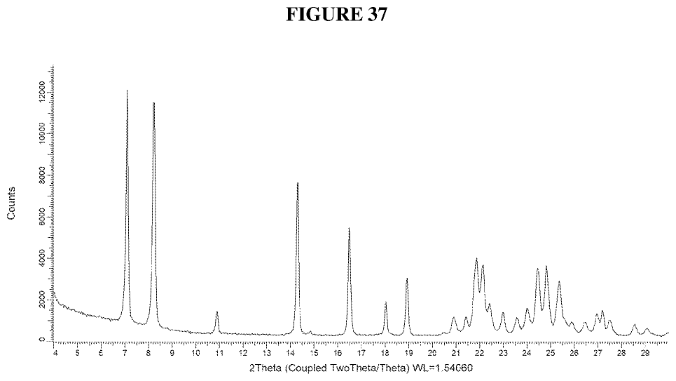

In one embodiment, the crystalline salt Form 3-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, fumaric acid salt, is characterized by a powder X-ray diffraction pattern that has a characteristic peak in degrees 2θ at about 8.2, is characterized by a powder X-ray diffraction pattern that has a characteristic peak in degrees 2θ at about 14.3, is characterized by a powder X-ray diffraction pattern that has a characteristic peak in degrees 2θ at about 16.5, is characterized by a powder X-ray diffraction pattern that has a characteristic peak in degrees 2θ at about 18.0, is characterized by a powder X-ray diffraction pattern that has a characteristic peak in degrees 2θ at about 18.9, is characterized by a powder X-ray diffraction pattern that has a characteristic peak in degrees 2θ at about 21.9, is characterized by a powder X-ray diffraction pattern that has a characteristic peak in degrees 2θ at about 22.1, is characterized by a powder X-ray diffraction pattern that has a characteristic peak in degrees 2θ at about 22.4, is characterized by a powder X-ray diffraction pattern that has a characteristic peak in degrees 2θ at about 24.5, is characterized by a powder X-ray diffraction pattern that has a characteristic peak in degrees 2θ at about 24.8, and/or is characterized by a powder X-ray diffraction pattern that has a characteristic peak in degrees 2θ at about 25.4. In yet another embodiment, crystalline salt Form 3-A is characterized by a powder X-ray diffraction pattern having at least one or more characteristic peaks in degrees 2θ at about 7.1, 8.2, 14.3, 16.5, 18.0, 18.9, 21.9, 22.1, 22.4, 24.5, 24.8, and 25.4. For example, a contemplated crystalline salt form has a powder X-ray diffraction pattern shown in FIG. 37. In one embodiment, the powder X-ray diffraction pattern of the crystalline form was obtained using Cu Kα radiation.

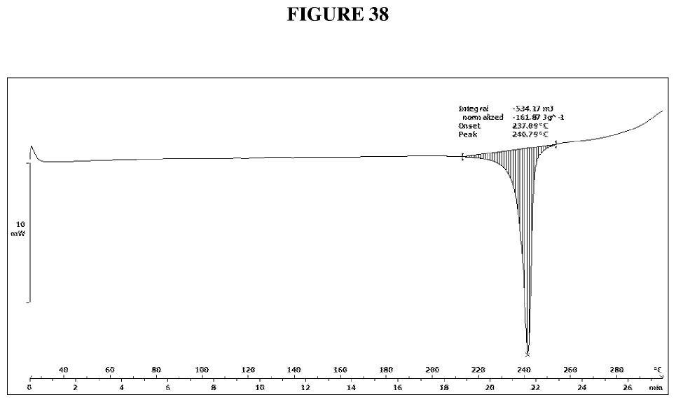

The contemplated crystalline Form 3-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, fumaric acid salt, may be characterized by a differential scanning calorimetry (DSC) profile showing a characteristic endotherm with an onset of about 237° C. and a peak of about 241° C. Form 3-A, for example, may be characterized by the differential scanning calorimetry profile shown in FIG. 38.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, characterized by a powder X-ray diffraction pattern having a characteristic peak in degrees 2θ at about 7.8 (referred to herein as “Form 5-A”).

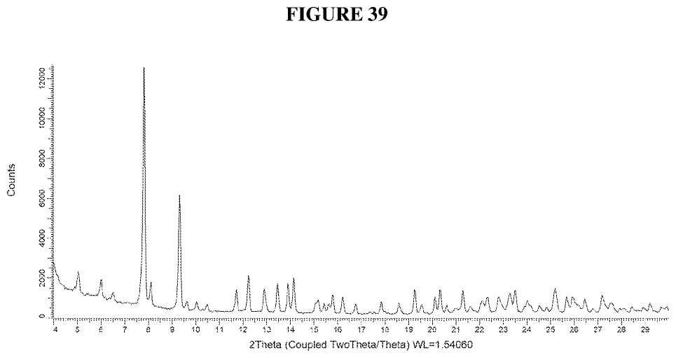

In one embodiment, the crystalline salt Form 5-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is characterized by a powder X-ray diffraction pattern that has a characteristic peak in degrees 2θ at about 5.0, is characterized by a powder X-ray diffraction pattern that has a characteristic peak in degrees 2θ at about 8.1, is characterized by a powder X-ray diffraction pattern that has a characteristic peak in degrees 2θ at about 9.3, is characterized by a powder X-ray diffraction pattern that has a characteristic peak in degrees 2θ at about 12.2, is characterized by a powder X-ray diffraction pattern that has a characteristic peak in degrees 2θ at about 13.5, is characterized by a powder X-ray diffraction pattern that has a characteristic peak in degrees 2θ at about 13.9, is characterized by a powder X-ray diffraction pattern that has a characteristic peak in degrees 2θ at about 14.1, is characterized by a powder X-ray diffraction pattern that has a characteristic peak in degrees 2θ at about 19.3, is characterized by a powder X-ray diffraction pattern that has a characteristic peak in degrees 2θ at about 20.3, is characterized by a powder X-ray diffraction pattern that has a characteristic peak in degrees 2θ at about 21.3, and/or is characterized by a powder X-ray diffraction pattern that has a characteristic peak in degrees 2θ at about 25.2. In yet another embodiment, crystalline salt Form 5-A is characterized by a powder X-ray diffraction pattern having at least one or more characteristic peaks in degrees 2θ at about 5.0, 7.8, 8.1, 9.3, 12.2, 13.5, 13.9, 14.1, 19.3, 20.3, 21.3 and 25.2. For example, a contemplated crystalline salt form has a powder X-ray diffraction pattern shown in FIG. 39. In one embodiment, the powder X-ray diffraction pattern of the crystalline form was obtained using Cu Kα radiation.

The contemplated crystalline Form 5-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloric acid salt, may be characterized by a thermogravimetric analysis (TGA) profile showing a mass loss of about 3.1 wt. % up to about 110° C. and a further mass loss of about 7.6 wt. % between about 110° C. to about 195° C. In some embodiments, crystalline Form 5-A may be characterized by a dynamic vapor sorption (DVS) profile showing a reversable total mass change of about 3.1 wt. % between about 2 to about 92% relative humidity (RH) at 25° C. In further embodiments, crystalline Form 5-A may be characterized by a Karl-Fischer titration profile showing a water content of about 10.7%. In other embodiments, crystalline Form 5-A may be characterized by optical microscopy showing a rod-like or plate-like morphology.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, characterized by a powder X-ray diffraction pattern having a characteristic peak in degrees 2θ at about 9.1 (referred to herein as “Form 5-B”), is disclosed herein.

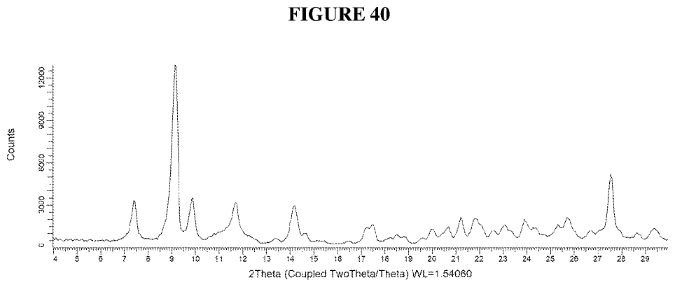

In one embodiment, the crystalline salt Form 5-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is characterized by a powder X-ray diffraction pattern that has a characteristic peak in degrees 2θ at about 7.4, is characterized by a powder X-ray diffraction pattern that has a characteristic peak in degrees 2θ at about 9.9, is characterized by a powder X-ray diffraction pattern that has a characteristic peak in degrees 2θ at about 11.7, is characterized by a powder X-ray diffraction pattern that has a characteristic peak in degrees 2θ at about 14.2, is characterized by a powder X-ray diffraction pattern that has a characteristic peak in degrees 2θ at about 17.5, is characterized by a powder X-ray diffraction pattern that has a characteristic peak in degrees 2θ at about 20.0, is characterized by a powder X-ray diffraction pattern that has a characteristic peak in degrees 2θ at about 21.2, is characterized by a powder X-ray diffraction pattern that has a characteristic peak in degrees 2θ at about 21.8, is characterized by a powder X-ray diffraction pattern that has a characteristic peak in degrees 2θ at about 23.9, is characterized by a powder X-ray diffraction pattern that has a characteristic peak in degrees 2θ at about 25.7, and/or is characterized by a powder X-ray diffraction pattern that has a characteristic peak in degrees 2θ at about 27.6. In yet another embodiment, crystalline salt Form 5-B is characterized by a powder X-ray diffraction pattern having at least one or more characteristic peaks in degrees 2θ at about 7.4, 9.1, 9.9, 11.7, 14.2, 17.5, 20.0, 21.2, 21.8, 23.9, 25.7, and 27.6. For example, a contemplated crystalline salt form has a powder X-ray diffraction pattern shown in FIG. 40. In one embodiment, the powder X-ray diffraction pattern of the crystalline form was obtained using Cu Kα radiation.

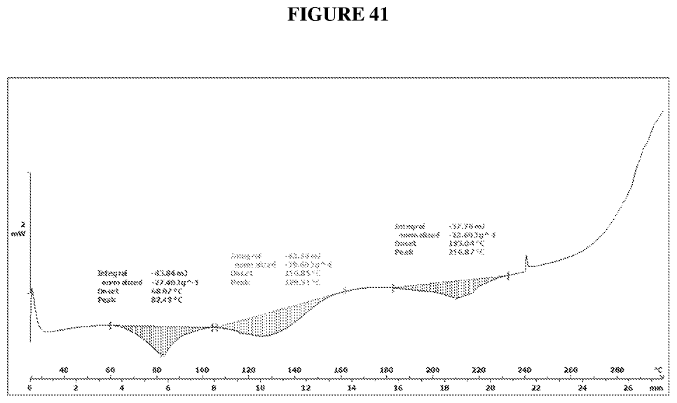

The contemplated crystalline Form 5-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, may be characterized by a differential scanning calorimetry (DSC) profile showing a characteristic endotherm with an onset of about 68° C. and a peak of about 82° C., a characteristic endotherm with an onset of about 111° C. and a peak of about 130° C., and a characteristic endotherm with an onset of about 193° C. and a peak of about 211° C. Form 5-B, for example, may be characterized by the differential scanning calorimetry profile shown in FIG. 41.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, characterized by a powder X-ray diffraction pattern having a characteristic peak in degrees 2θ at about 21.5 (referred to herein as “Form 5-C”), is disclosed herein.

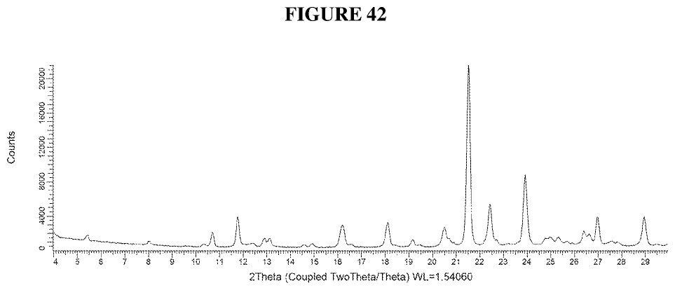

In one embodiment, the crystalline salt Form 5-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is characterized by a powder X-ray diffraction pattern that has a characteristic peak in degrees 2θ at about 10.7, is characterized by a powder X-ray diffraction pattern that has a characteristic peak in degrees 2θ at about 11.8, is characterized by a powder X-ray diffraction pattern that has a characteristic peak in degrees 2θ at about 12.9, is characterized by a powder X-ray diffraction pattern that has a characteristic peak in degrees 2θ at about 16.2, is characterized by a powder X-ray diffraction pattern that has a characteristic peak in degrees 2θ at about 18.1, is characterized by a powder X-ray diffraction pattern that has a characteristic peak in degrees 2θ at about 20.5, is characterized by a powder X-ray diffraction pattern that has a characteristic peak in degrees 2θ at about 22.4, is characterized by a powder X-ray diffraction pattern that has a characteristic peak in degrees 2θ at about 23.9, is characterized by a powder X-ray diffraction pattern that has a characteristic peak in degrees 2θ at about 26.4, is characterized by a powder X-ray diffraction pattern that has a characteristic peak in degrees 2θ at about 27.0, and/or is characterized by a powder X-ray diffraction pattern that has a characteristic peak in degrees 2θ at about 28.9. In yet another embodiment, crystalline salt Form 5-C is characterized by a powder X-ray diffraction pattern having at least one or more characteristic peaks in degrees 2θ at about 10.7, 11.8, 12.9, 16.2, 18.1, 20.5, 21.5, 22.4, 23.9, 26.4, 27.0, and 28.9. For example, a contemplated crystalline salt form has a powder X-ray diffraction pattern shown in FIG. 42. In one embodiment, the powder X-ray diffraction pattern of the crystalline form was obtained using Cu Kα radiation.

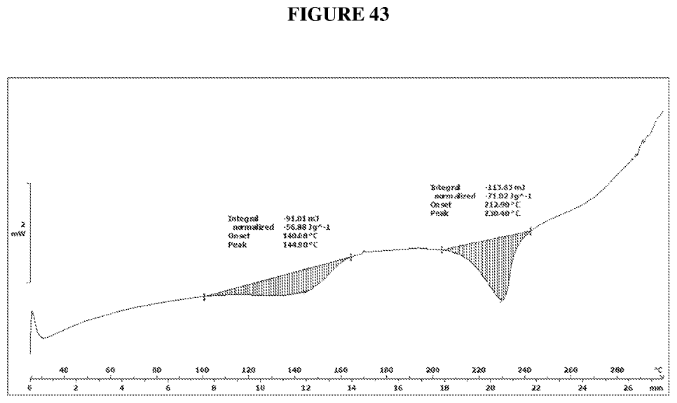

The contemplated crystalline Form 5-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, may be characterized by a differential scanning calorimetry (DSC) profile showing a characteristic endotherm with an onset of about 140° C. and a peak of about 145° C., and a characteristic endotherm with an onset of about 213° C. and a peak of about 230° C. Form 5-C, for example, may be characterized by the differential scanning calorimetry profile shown in FIG. 43.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, characterized by a powder X-ray diffraction pattern having a characteristic peak in degrees 2θ at about 8.4 (referred to herein as “Form 5-D”), is disclosed herein.

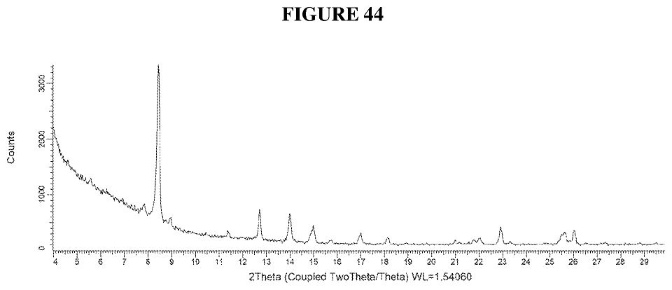

In one embodiment, the crystalline salt Form 5-D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is characterized by a powder X-ray diffraction pattern that has a characteristic peak in degrees 2θ at about 7.8, is characterized by a powder X-ray diffraction pattern that has a characteristic peak in degrees 2θ at about 12.7, is characterized by a powder X-ray diffraction pattern that has a characteristic peak in degrees 2θ at about 14.0, is characterized by a powder X-ray diffraction pattern that has a characteristic peak in degrees 2θ at about 15.0, is characterized by a powder X-ray diffraction pattern that has a characteristic peak in degrees 2θ at about 17.0, is characterized by a powder X-ray diffraction pattern that has a characteristic peak in degrees 2θ at about 22.9, is characterized by a powder X-ray diffraction pattern that has a characteristic peak in degrees 2θ at about 25.6, and/or is characterized by a powder X-ray diffraction pattern that has a characteristic peak in degrees 2θ at about 26.0. In yet another embodiment, crystalline salt Form 5-D is characterized by a powder X-ray diffraction pattern having at least one or more characteristic peaks in degrees 2θ at about 7.8, 8.4, 12.7, 14.0, 15.0, 17.0, 22.9, 25.6, and 26.0. For example, a contemplated crystalline salt form has a powder X-ray diffraction pattern shown in FIG. 44. In one embodiment, the powder X-ray diffraction pattern of the crystalline form was obtained using Cu Kα radiation.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt, characterized by a powder X-ray diffraction pattern having a characteristic peak in degrees 2θ at about 21.5 (referred to herein as “Form 7-A”).

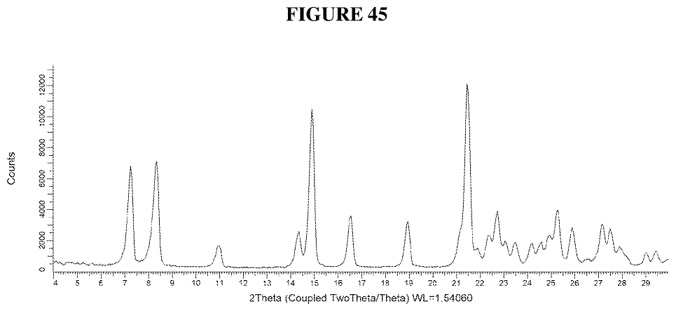

In one embodiment, the crystalline salt Form 7-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt, is characterized by a powder X-ray diffraction pattern that has a characteristic peak in degrees 2θ at about 7.2, is characterized by a powder X-ray diffraction pattern that has a characteristic peak in degrees 2θ at about 8.3, is characterized by a powder X-ray diffraction pattern that has a characteristic peak in degrees 2θ at about 14.3, is characterized by a powder X-ray diffraction pattern that has a characteristic peak in degrees 2θ at about 14.9, is characterized by a powder X-ray diffraction pattern that has a characteristic peak in degrees 2θ at about 16.5, is characterized by a powder X-ray diffraction pattern that has a characteristic peak in degrees 2θ at about 18.9, is characterized by a powder X-ray diffraction pattern that has a characteristic peak in degrees 2θ at about 22.7, is characterized by a powder X-ray diffraction pattern that has a characteristic peak in degrees 2θ at about 25.3, is characterized by a powder X-ray diffraction pattern that has a characteristic peak in degrees 2θ at about 25.9, is characterized by a powder X-ray diffraction pattern that has a characteristic peak in degrees 2θ at about 27.2, and/or is characterized by a powder X-ray diffraction pattern that has a characteristic peak in degrees 2θ at about 27.5. In yet another embodiment, crystalline salt Form 7-A is characterized by a powder X-ray diffraction pattern having at least one or more characteristic peaks in degrees 2θ at about 7.2, 8.3, 14.3, 14.9, 16.5, 18.9, 21.5, 22.7, 25.3, 25.9, 27.2, and 27.5. For example, a contemplated crystalline salt form has a powder X-ray diffraction pattern shown in FIG. 45. In one embodiment, the powder X-ray diffraction pattern of the crystalline form was obtained using Cu Kα radiation.

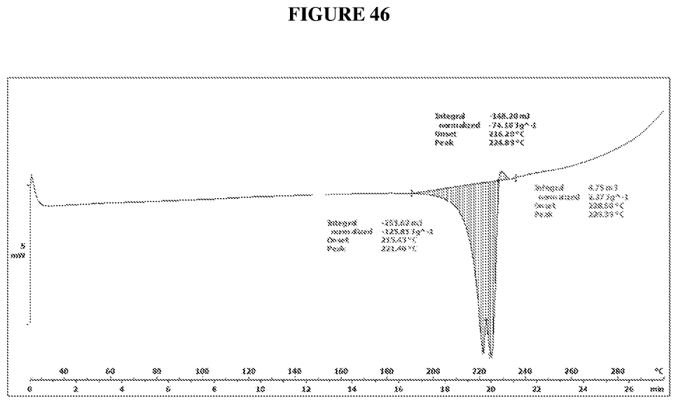

The contemplated crystalline Form 7-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt, may be characterized by a differential scanning calorimetry (DSC) profile showing a characteristic endotherm with an onset of about 215° C. and a peak of about 221° C., and a characteristic endotherm with an onset of about 216° C. and a peak of about 225° C. Form 7-A, for example, may be characterized by the differential scanning calorimetry profile shown in FIG. 46.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, characterized by a powder X-ray diffraction pattern having a characteristic peak in degrees 2θ at about 15.0 (referred to herein as “Form 8-A”).

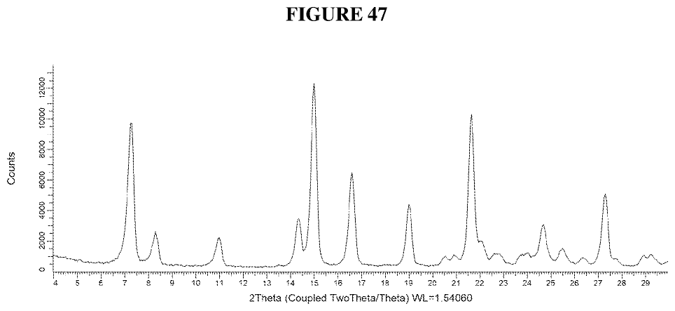

In one embodiment, the crystalline salt Form 8-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, is characterized by a powder X-ray diffraction pattern that has a characteristic peak in degrees 2θ at about 7.3, is characterized by a powder X-ray diffraction pattern that has a characteristic peak in degrees 2θ at about 8.3, is characterized by a powder X-ray diffraction pattern that has a characteristic peak in degrees 2θ at about 11.0, is characterized by a powder X-ray diffraction pattern that has a characteristic peak in degrees 2θ at about 14.3, is characterized by a powder X-ray diffraction pattern that has a characteristic peak in degrees 2θ at about 16.6, is characterized by a powder X-ray diffraction pattern that has a characteristic peak in degrees 2θ at about 19.0, is characterized by a powder X-ray diffraction pattern that has a characteristic peak in degrees 2θ at about 21.6, is characterized by a powder X-ray diffraction pattern that has a characteristic peak in degrees 2θ at about 22.0, is characterized by a powder X-ray diffraction pattern that has a characteristic peak in degrees 2θ at about 24.7, is characterized by a powder X-ray diffraction pattern that has a characteristic peak in degrees 2θ at about 25.5, and/or is characterized by a powder X-ray diffraction pattern that has a characteristic peak in degrees 2θ at about 27.3. In yet another embodiment, crystalline salt Form 8-A is characterized by a powder X-ray diffraction pattern having at least one or more characteristic peaks in degrees 2θ at about 7.3, 8.3, 11.0, 14.3, 15.0, 16.6, 19.0, 21.6, 22.0, 24.7, 25.5, and 27.3. For example, a contemplated crystalline salt form has a powder X-ray diffraction pattern shown in FIG. 47. In one embodiment, the powder X-ray diffraction pattern of the crystalline form was obtained using Cu Kα radiation.

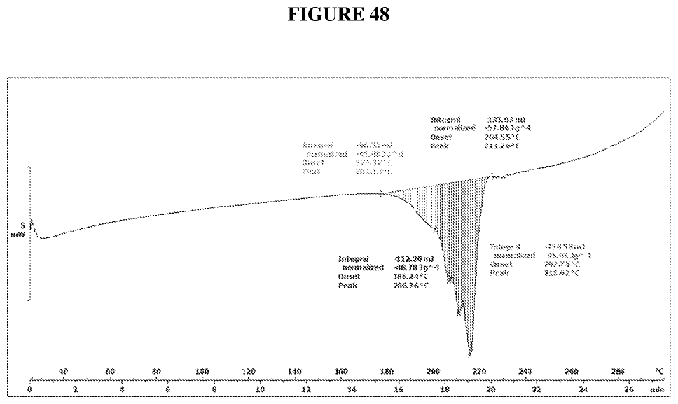

The contemplated crystalline Form 8-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, may be characterized by a differential scanning calorimetry (DSC) profile showing a characteristic endotherm with an onset of about 177° C. and a peak of about 201° C., a characteristic endotherm with an onset of about 186° C. and a peak of about 207° C., a characteristic endotherm with an onset of about 205° C. and a peak of about 211° C., and a characteristic endotherm with an onset of about 208° C. and a peak of about 216° C. Form 8-A, for example, may be characterized by the differential scanning calorimetry profile shown in FIG. 48.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, characterized by a powder X-ray diffraction pattern having a characteristic peak in degrees 2θ at about 5.9 (referred to herein as “Form 8-B”), is disclosed herein.

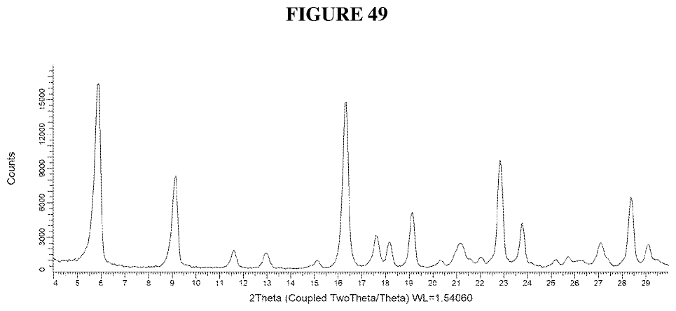

In one embodiment, the crystalline salt Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, is characterized by a powder X-ray diffraction pattern that has a characteristic peak in degrees 2θ at about 9.1, is characterized by a powder X-ray diffraction pattern that has a characteristic peak in degrees 2θ at about 11.6, is characterized by a powder X-ray diffraction pattern that has a characteristic peak in degrees 2θ at about 16.3, is characterized by a powder X-ray diffraction pattern that has a characteristic peak in degrees 2θ at about 17.6, is characterized by a powder X-ray diffraction pattern that has a characteristic peak in degrees 2θ at about 18.2, is characterized by a powder X-ray diffraction pattern that has a characteristic peak in degrees 2θ at about 19.1, is characterized by a powder X-ray diffraction pattern that has a characteristic peak in degrees 2θ at about 21.2, is characterized by a powder X-ray diffraction pattern that has a characteristic peak in degrees 2θ at about 22.9, is characterized by a powder X-ray diffraction pattern that has a characteristic peak in degrees 2θ at about 23.8, is characterized by a powder X-ray diffraction pattern that has a characteristic peak in degrees 2θ at about 27.1, and/or is characterized by a powder X-ray diffraction pattern that has a characteristic peak in degrees 2θ at about 28.4. In yet another embodiment, crystalline salt Form 8-B is characterized by a powder X-ray diffraction pattern having at least one or more characteristic peaks in degrees 2θ at about 5.9, 9.1, 11.6, 16.3, 17.6, 18.2, 19.1, 21.2, 22.9, 23.8, 27.1, and 28.4. For example, a contemplated crystalline salt form has a powder X-ray diffraction pattern shown in FIG. 49. In one embodiment, the powder X-ray diffraction pattern of the crystalline form was obtained using Cu Kα radiation.

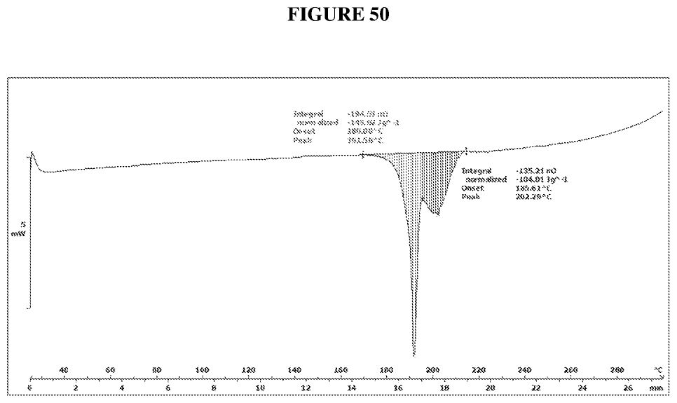

The contemplated crystalline Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, may be characterized by a differential scanning calorimetry (DSC) profile showing a characteristic endotherm with an onset of about 189° C. and a peak of about 192° C., and a characteristic endotherm with an onset of about 186° C. and a peak of about 202° C. Form 8-B, for example, may be characterized by the differential scanning calorimetry profile shown in FIG. 50.

The contemplated crystalline Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, may be characterized by a thermogravimetric analysis (TGA) profile showing a mass loss of about 0.46 wt. % up to about 175° C. In some embodiments, crystalline Form 8-B may be characterized by a dynamic vapor sorption (DVS) profile showing a reversable total mass change of about 0.82 wt. % between about 2 to about 92% relative humidity (RH) at 25° C.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, characterized by a powder X-ray diffraction pattern having a characteristic peak in degrees 2θ at about 9.5 (referred to herein as “Form 9-A”).

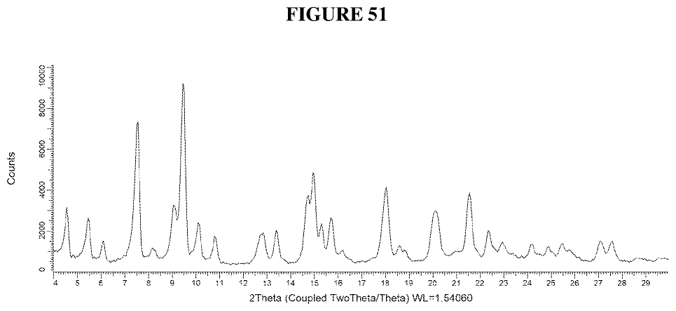

In one embodiment, the crystalline salt Form 9-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is characterized by a powder X-ray diffraction pattern that has a characteristic peak in degrees 2θ at about 4.5, is characterized by a powder X-ray diffraction pattern that has a characteristic peak in degrees 2θ at about 5.4, is characterized by a powder X-ray diffraction pattern that has a characteristic peak in degrees 2θ at about 7.5, is characterized by a powder X-ray diffraction pattern that has a characteristic peak in degrees 2θ at about 9.1, is characterized by a powder X-ray diffraction pattern that has a characteristic peak in degrees 2θ at about 10.1, is characterized by a powder X-ray diffraction pattern that has a characteristic peak in degrees 2θ at about 14.7, is characterized by a powder X-ray diffraction pattern that has a characteristic peak in degrees 2θ at about 15.0, is characterized by a powder X-ray diffraction pattern that has a characteristic peak in degrees 2θ at about 15.7, is characterized by a powder X-ray diffraction pattern that has a characteristic peak in degrees 2θ at about 18.0, is characterized by a powder X-ray diffraction pattern that has a characteristic peak in degrees 2θ at about 20.1, and/or is characterized by a powder X-ray diffraction pattern that has a characteristic peak in degrees 2θ at about 21.5. In yet another embodiment, crystalline salt Form 9-A is characterized by a powder X-ray diffraction pattern having at least one or more characteristic peaks in degrees 2θ at about 4.5, 5.4, 7.5, 9.1, 9.5, 10.1, 14.7, 15.0, 15.7, 18.0, 20.1, and 21.5. For example, a contemplated crystalline salt form has a powder X-ray diffraction pattern shown in FIG. 51. In one embodiment, the powder X-ray diffraction pattern of the crystalline form was obtained using Cu Kα radiation.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, characterized by a powder X-ray diffraction pattern having a characteristic peak in degrees 2θ at about 8.2 (referred to herein as “Form 9-B”), is disclosed herein.

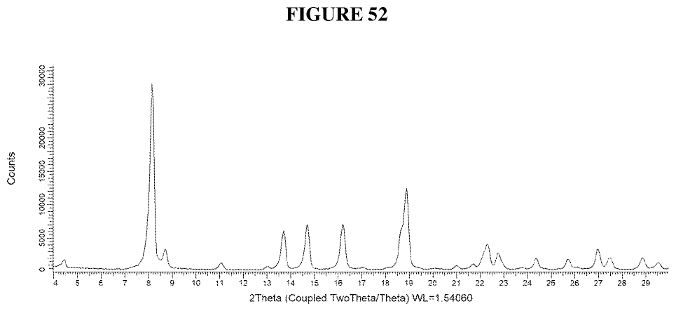

In one embodiment, the crystalline salt Form 9-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is characterized by a powder X-ray diffraction pattern that has a characteristic peak in degrees 2θ at about 8.7, is characterized by a powder X-ray diffraction pattern that has a characteristic peak in degrees 2θ at about 13.7, is characterized by a powder X-ray diffraction pattern that has a characteristic peak in degrees 2θ at about 14.7, is characterized by a powder X-ray diffraction pattern that has a characteristic peak in degrees 2θ at about 16.2, is characterized by a powder X-ray diffraction pattern that has a characteristic peak in degrees 2θ at about 18.9, is characterized by a powder X-ray diffraction pattern that has a characteristic peak in degrees 2θ at about 22.3, is characterized by a powder X-ray diffraction pattern that has a characteristic peak in degrees 2θ at about 22.8, is characterized by a powder X-ray diffraction pattern that has a characteristic peak in degrees 2θ at about 24.4, is characterized by a powder X-ray diffraction pattern that has a characteristic peak in degrees 2θ at about 27.0, is characterized by a powder X-ray diffraction pattern that has a characteristic peak in degrees 2θ at about 27.5, and/or is characterized by a powder X-ray diffraction pattern that has a characteristic peak in degrees 2θ at about 28.9. In yet another embodiment, crystalline salt Form 9-B is characterized by a powder X-ray diffraction pattern having at least one or more characteristic peaks in degrees 2θ at about 8.2, 8.7, 13.7, 14.7, 16.2, 18.9, 22.3, 22.8, 24.4, 27.0, 27.5, and 28.9. For example, a contemplated crystalline salt form has a powder X-ray diffraction pattern shown in FIG. 52. In one embodiment, the powder X-ray diffraction pattern of the crystalline form was obtained using Cu Kα radiation.

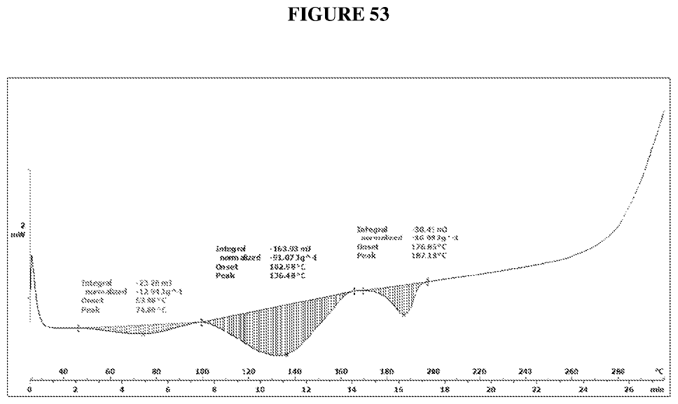

The contemplated crystalline Form 9-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, may be characterized by a differential scanning calorimetry (DSC) profile showing a characteristic endotherm with an onset of about 51° C. and a peak of about 74° C., and a characteristic endotherm with an onset of about 177° C. and a peak of about 187° C. Form 9-B, for example, may be characterized by the differential scanning calorimetry profile shown in FIG. 53.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, characterized by a powder X-ray diffraction pattern having a characteristic peak in degrees 2θ at about 9.5 (referred to herein as “Form 9-C”), is disclosed herein.

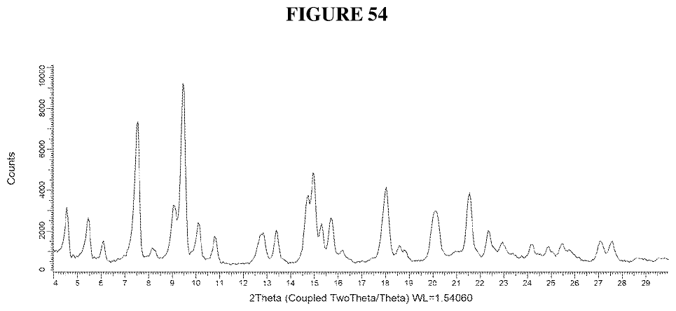

In one embodiment, the crystalline salt Form 9-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is characterized by a powder X-ray diffraction pattern that has a characteristic peak in degrees 2θ at about 4.5, is characterized by a powder X-ray diffraction pattern that has a characteristic peak in degrees 2θ at about 7.6, is characterized by a powder X-ray diffraction pattern that has a characteristic peak in degrees 2θ at about 9.2, is characterized by a powder X-ray diffraction pattern that has a characteristic peak in degrees 2θ at about 10.2, is characterized by a powder X-ray diffraction pattern that has a characteristic peak in degrees 2θ at about 10.9, is characterized by a powder X-ray diffraction pattern that has a characteristic peak in degrees 2θ at about 14.8, is characterized by a powder X-ray diffraction pattern that has a characteristic peak in degrees 2θ at about 15.0, is characterized by a powder X-ray diffraction pattern that has a characteristic peak in degrees 2θ at about 15.9, is characterized by a powder X-ray diffraction pattern that has a characteristic peak in degrees 2θ at about 18.2, is characterized by a powder X-ray diffraction pattern that has a characteristic peak in degrees 2θ at about 20.4, and/or is characterized by a powder X-ray diffraction pattern that has a characteristic peak in degrees 2θ at about 21.8. In yet another embodiment, crystalline salt Form 9-C is characterized by a powder X-ray diffraction pattern having at least one or more characteristic peaks in degrees 2θ at about 4.5, 7.6, 9.2, 9.5, 10.2, 10.9, 14.8, 15.0, 15.9, 18.2, 20.4, and 21.8. For example, a contemplated crystalline salt form has a powder X-ray diffraction pattern shown in FIG. 54. In one embodiment, the powder X-ray diffraction pattern of the crystalline form was obtained using Cu Kα radiation.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, characterized by a powder X-ray diffraction pattern having a characteristic peak in degrees 2θ at about 12.3 (referred to herein as “Form 10-A”).

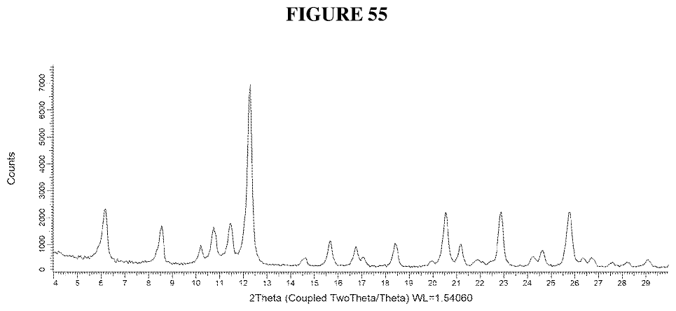

In one embodiment, the crystalline salt Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, is characterized by a powder X-ray diffraction pattern that has a characteristic peak in degrees 2θ at about 6.2, is characterized by a powder X-ray diffraction pattern that has a characteristic peak in degrees 2θ at about 8.5, is characterized by a powder X-ray diffraction pattern that has a characteristic peak in degrees 2θ at about 10.2, is characterized by a powder X-ray diffraction pattern that has a characteristic peak in degrees 2θ at about 10.8, is characterized by a powder X-ray diffraction pattern that has a characteristic peak in degrees 2θ at about 11.5, is characterized by a powder X-ray diffraction pattern that has a characteristic peak in degrees 2θ at about 15.7, is characterized by a powder X-ray diffraction pattern that has a characteristic peak in degrees 2θ at about 18.4, is characterized by a powder X-ray diffraction pattern that has a characteristic peak in degrees 2θ at about 20.5, is characterized by a powder X-ray diffraction pattern that has a characteristic peak in degrees 2θ at about 21.2, is characterized by a powder X-ray diffraction pattern that has a characteristic peak in degrees 2θ at about 22.9, and/or is characterized by a powder X-ray diffraction pattern that has a characteristic peak in degrees 2θ at about 25.8. In yet another embodiment, crystalline salt Form 10-A is characterized by a powder X-ray diffraction pattern having at least one or more characteristic peaks in degrees 2θ at about 6.2, 8.5, 10.2, 10.8, 11.5, 12.3, 15.7, 18.4, 20.5, 21.2, 22.9, and 25.8. For example, a contemplated crystalline salt form has a powder X-ray diffraction pattern shown in FIG. 55. In one embodiment, the powder X-ray diffraction pattern of the crystalline form was obtained using Cu Kα radiation.

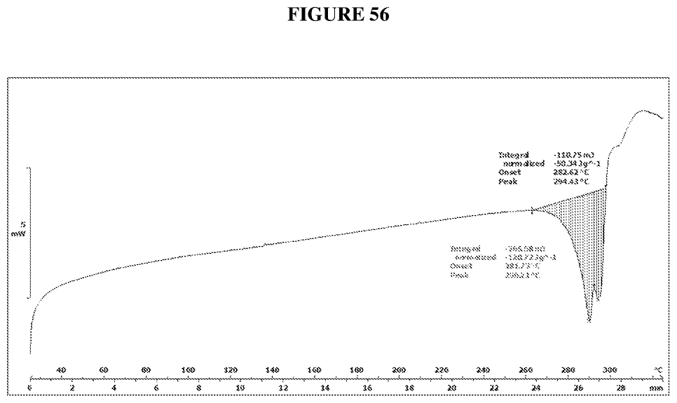

The contemplated crystalline Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, may be characterized by a differential scanning calorimetry (DSC) profile showing a characteristic endotherm with an onset of about 282° C. and a peak of about 290° C., and a characteristic endotherm with an onset of about 283° C. and a peak of about 294° C. Form 10-A, for example, may be characterized by the differential scanning calorimetry profile shown in FIG. 56.

The contemplated crystalline Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, may be characterized by a thermogravimetric analysis (TGA) profile showing a mass loss of about 0.22 wt. %. In some embodiments, crystalline Form 10-A may be characterized by a dynamic vapor sorption (DVS) profile showing a reversable total mass change of about 1.4 wt. % between about 2 to about 92% relative humidity (RH) at 25° C.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, pyruvic acid salt, characterized by a powder X-ray diffraction pattern having a characteristic peak in degrees 2θ at about 7.5 (referred to herein as “Form 11-A”).

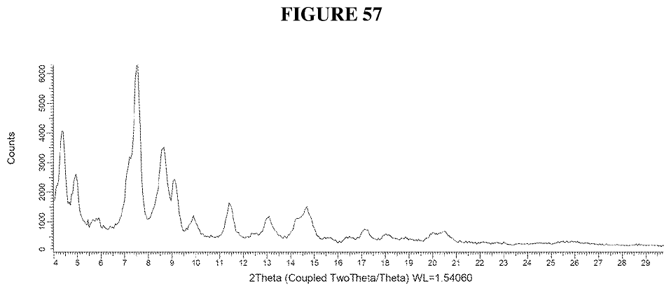

In one embodiment, the crystalline salt Form 11-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, pyruvic acid salt, is characterized by a powder X-ray diffraction pattern that has a characteristic peak in degrees 2θ at about 4.4, is characterized by a powder X-ray diffraction pattern that has a characteristic peak in degrees 2θ at about 4.9, is characterized by a powder X-ray diffraction pattern that has a characteristic peak in degrees 2θ at about 7.2, is characterized by a powder X-ray diffraction pattern that has a characteristic peak in degrees 2θ at about 8.6 is characterized by a powder X-ray diffraction pattern that has a characteristic peak in degrees 2θ at about 9.1, is characterized by a powder X-ray diffraction pattern that has a characteristic peak in degrees 2θ at about 9.9, is characterized by a powder X-ray diffraction pattern that has a characteristic peak in degrees 2θ at about 11.4, is characterized by a powder X-ray diffraction pattern that has a characteristic peak in degrees 2θ at about 13.1, is characterized by a powder X-ray diffraction pattern that has a characteristic peak in degrees 2θ at about 14.5, is characterized by a powder X-ray diffraction pattern that has a characteristic peak in degrees 2θ at about 14.7, and/or is characterized by a powder X-ray diffraction pattern that has a characteristic peak in degrees 2θ at about 17.2. In yet another embodiment, crystalline salt Form 11-A is characterized by a powder X-ray diffraction pattern having at least one or more characteristic peaks in degrees 2θ at about 4.4, 4.9, 7.2, 7.5, 8.6, 9.1, 9.9, 11.4, 13.1, 14.5, 14.7, and 17.2. For example, a contemplated crystalline salt form has a powder X-ray diffraction pattern shown in FIG. 57. In one embodiment, the powder X-ray diffraction pattern of the crystalline form was obtained using Cu Kα radiation.

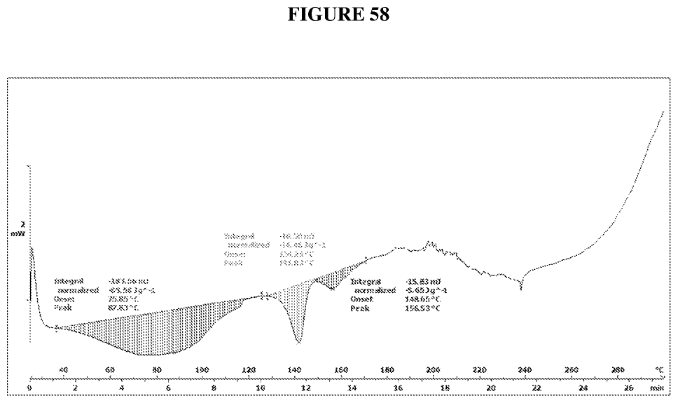

The contemplated crystalline Form 11-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, pyruvic acid salt, may be characterized by a differential scanning calorimetry (DSC) profile showing a characteristic endotherm with an onset of about 76° C. and a peak of about 88° C., a characteristic endotherm with an onset of about 134° C. and a peak of about 142° C., and a characteristic endotherm with an onset of about 149° C. and a peak of about 157° C. Form 11-A, for example, may be characterized by the differential scanning calorimetry profile shown in FIG. 58.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, sulfuric acid salt, characterized by a powder X-ray diffraction pattern having a characteristic peak in degrees 2θ at about 5.9 (referred to herein as “Form 12-A”).

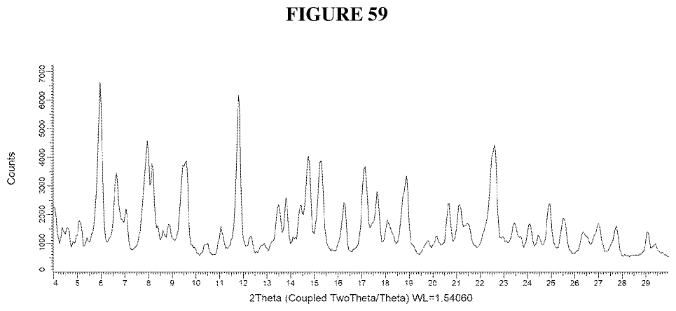

In one embodiment, the crystalline salt Form 12-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, sulfuric acid salt, is characterized by a powder X-ray diffraction pattern that has a characteristic peak in degrees 2θ at about 6.6, is characterized by a powder X-ray diffraction pattern that has a characteristic peak in degrees 2θ at about 7.9, is characterized by a powder X-ray diffraction pattern that has a characteristic peak in degrees 2θ at about 8.2, is characterized by a powder X-ray diffraction pattern that has a characteristic peak in degrees 2θ at about 9.6 is characterized by a powder X-ray diffraction pattern that has a characteristic peak in degrees 2θ at about 11.8, is characterized by a powder X-ray diffraction pattern that has a characteristic peak in degrees 2θ at about 14.7, is characterized by a powder X-ray diffraction pattern that has a characteristic peak in degrees 2θ at about 15.3, is characterized by a powder X-ray diffraction pattern that has a characteristic peak in degrees 2θ at about 17.1, is characterized by a powder X-ray diffraction pattern that has a characteristic peak in degrees 2θ at about 17.7, is characterized by a powder X-ray diffraction pattern that has a characteristic peak in degrees 2θ at about 18.9, and/or is characterized by a powder X-ray diffraction pattern that has a characteristic peak in degrees 2θ at about 22.6. In yet another embodiment, crystalline salt Form 12-A is characterized by a powder X-ray diffraction pattern having at least one or more characteristic peaks in degrees 2θ at about 5.9, 6.6, 7.9, 8.2, 9.6, 11.8, 14.7, 15.3, 17.1, 17.7, 18.9, and 22.6. For example, a contemplated crystalline salt form has a powder X-ray diffraction pattern shown in FIG. 59. In one embodiment, the powder X-ray diffraction pattern of the crystalline form was obtained using Cu Kα radiation.

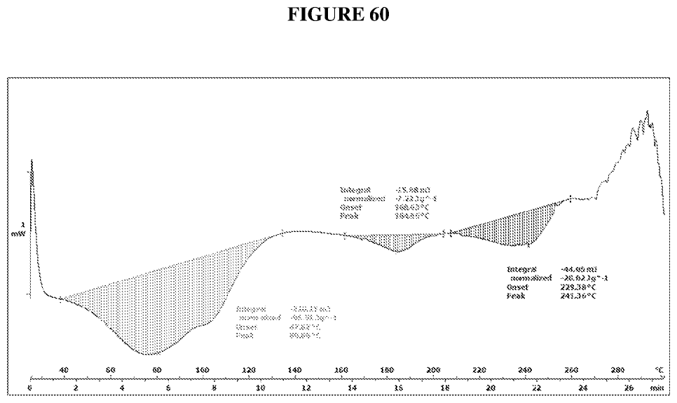

The contemplated crystalline Form 12-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, sulfuric acid salt, may be characterized by a differential scanning calorimetry (DSC) profile showing a characteristic endotherm with an onset of about 48° C. and a peak of about 81° C., a characteristic endotherm with an onset of about 169° C. and a peak of about 185° C., and a characteristic endotherm with an onset of about 229° C. and a peak of about 241° C. Form 12-A, for example, may be characterized by the differential scanning calorimetry profile shown in FIG. 60.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, characterized by a powder X-ray diffraction pattern having a characteristic peak in degrees 2θ at about 15.9 (referred to herein as “Form 13-A”).

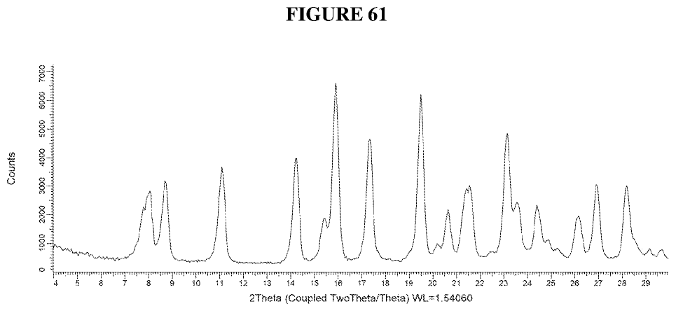

In one embodiment, the crystalline salt Form 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, is characterized by a powder X-ray diffraction pattern that has a characteristic peak in degrees 2θ at about 8.0, is characterized by a powder X-ray diffraction pattern that has a characteristic peak in degrees 2θ at about 8.7, is characterized by a powder X-ray diffraction pattern that has a characteristic peak in degrees 2θ at about 11.1, is characterized by a powder X-ray diffraction pattern that has a characteristic peak in degrees 2θ at about 14.2 is characterized by a powder X-ray diffraction pattern that has a characteristic peak in degrees 2θ at about 17.3, is characterized by a powder X-ray diffraction pattern that has a characteristic peak in degrees 2θ at about 19.5, is characterized by a powder X-ray diffraction pattern that has a characteristic peak in degrees 2θ at about 21.6, is characterized by a powder X-ray diffraction pattern that has a characteristic peak in degrees 2θ at about 23.1, is characterized by a powder X-ray diffraction pattern that has a characteristic peak in degrees 2θ at about 23.6, is characterized by a powder X-ray diffraction pattern that has a characteristic peak in degrees 2θ at about 26.9, and/or is characterized by a powder X-ray diffraction pattern that has a characteristic peak in degrees 2θ at about 28.2. In yet another embodiment, crystalline salt Form 13-A is characterized by a powder X-ray diffraction pattern having at least one or more characteristic peaks in degrees 2θ at about 8.0, 8.7, 11.1, 14.2, 15.9, 17.3, 19.5, 21.6, 23.1, 23.6, 26.9, and 28.2. For example, a contemplated crystalline salt form has a powder X-ray diffraction pattern shown in FIG. 61. In one embodiment, the powder X-ray diffraction pattern of the crystalline form was obtained using Cu Kα radiation.

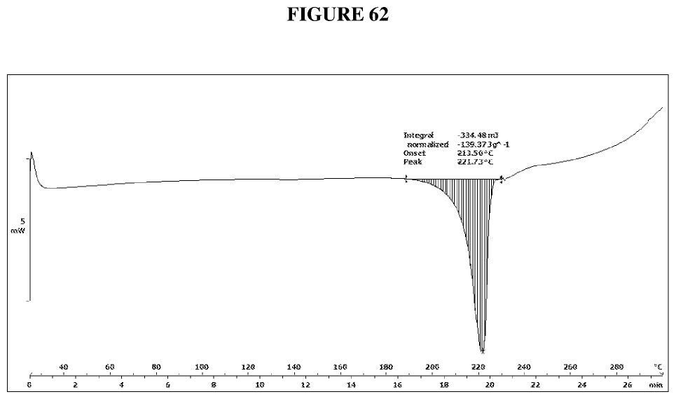

The contemplated crystalline Form 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, may be characterized by a differential scanning calorimetry (DSC) profile showing a characteristic endotherm with an onset of about 213° C. and a peak of about 222° C. Form 13-A, for example, may be characterized by the differential scanning calorimetry profile shown in FIG. 62.

The contemplated crystalline 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, may be characterized by a thermogravimetric analysis (TGA) profile showing a mass loss of about 0.63 wt. % up to about 185° C. In some embodiments, crystalline Form 13-A may be characterized by a dynamic vapor sorption (DVS) profile showing a reversable total mass change of about 0.97 wt. % between about 2 to about 92% relative humidity (RH) at 25° C.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, characterized by a powder X-ray diffraction pattern having a characteristic peak in degrees 2θ at about 16.9 (referred to herein as “Form 13-B”), is disclosed herein.

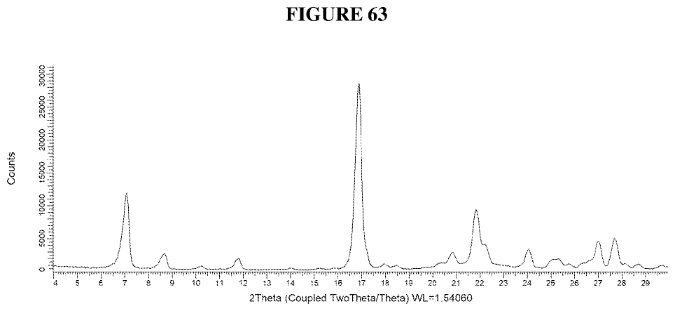

In one embodiment, the crystalline salt Form 13-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, is characterized by a powder X-ray diffraction pattern that has a characteristic peak in degrees 2θ at about 7.1, is characterized by a powder X-ray diffraction pattern that has a characteristic peak in degrees 2θ at about 8.7, is characterized by a powder X-ray diffraction pattern that has a characteristic peak in degrees 2θ at about 11.8, is characterized by a powder X-ray diffraction pattern that has a characteristic peak in degrees 2θ at about 20.8, is characterized by a powder X-ray diffraction pattern that has a characteristic peak in degrees 2θ at about 21.8, is characterized by a powder X-ray diffraction pattern that has a characteristic peak in degrees 2θ at about 22.2, is characterized by a powder X-ray diffraction pattern that has a characteristic peak in degrees 2θ at about 24.1, is characterized by a powder X-ray diffraction pattern that has a characteristic peak in degrees 2θ at about 25.1, is characterized by a powder X-ray diffraction pattern that has a characteristic peak in degrees 2θ at about 25.3, is characterized by a powder X-ray diffraction pattern that has a characteristic peak in degrees 2θ at about 27.0, and/or is characterized by a powder X-ray diffraction pattern that has a characteristic peak in degrees 2θ at about 27.7. In yet another embodiment, crystalline salt Form 13-B is characterized by a powder X-ray diffraction pattern having at least one or more characteristic peaks in degrees 2θ at about 7.1, 8.7, 11.8, 16.9, 20.8, 21.8, 22.2, 24.1, 25.1, 25.3, 27.0, and 27.7. For example, a contemplated crystalline salt form has a powder X-ray diffraction pattern shown in FIG. 63. In one embodiment, the powder X-ray diffraction pattern of the crystalline form was obtained using Cu Kα radiation.

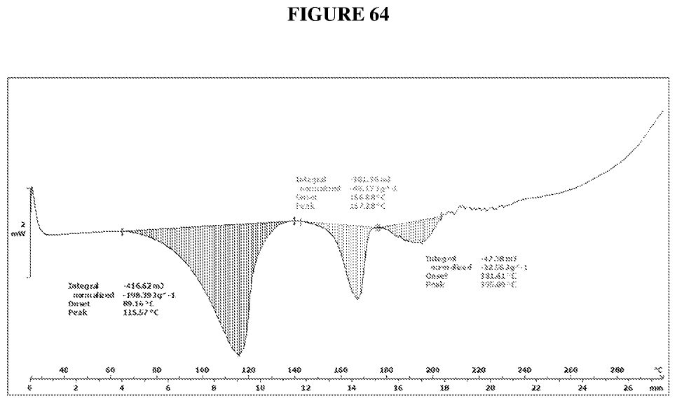

The contemplated crystalline Form 13-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, may be characterized by a differential scanning calorimetry (DSC) profile showing a characteristic endotherm with an onset of about 89° C. and a peak of about 115° C., a characteristic endotherm with an onset of about 157° C. and a peak of about 167° C., and a characteristic endotherm with an onset of about 181° C. and a peak of about 195° C. Form 13-B, for example, may be characterized by the differential scanning calorimetry profile shown in FIG. 64.

In another embodiment, disclosed herein is a pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, characterized by a powder X-ray diffraction pattern having a characteristic peak in degrees 2θ at about 17.5 (referred to herein as “Form 14-A”).

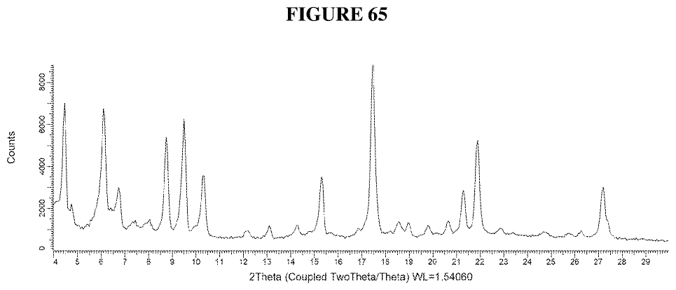

In one embodiment, the crystalline salt Form 14-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, is characterized by a powder X-ray diffraction pattern that has a characteristic peak in degrees 2θ at about 4.4, is characterized by a powder X-ray diffraction pattern that has a characteristic peak in degrees 2θ at about 6.1, is characterized by a powder X-ray diffraction pattern that has a characteristic peak in degrees 2θ at about 6.4, is characterized by a powder X-ray diffraction pattern that has a characteristic peak in degrees 2θ at about 6.7 is characterized by a powder X-ray diffraction pattern that has a characteristic peak in degrees 2θ at about 8.8, is characterized by a powder X-ray diffraction pattern that has a characteristic peak in degrees 2θ at about 9.5, is characterized by a powder X-ray diffraction pattern that has a characteristic peak in degrees 2θ at about 10.3, is characterized by a powder X-ray diffraction pattern that has a characteristic peak in degrees 2θ at about 15.3, is characterized by a powder X-ray diffraction pattern that has a characteristic peak in degrees 2θ at about 21.3, is characterized by a powder X-ray diffraction pattern that has a characteristic peak in degrees 2θ at about 21.9, and/or is characterized by a powder X-ray diffraction pattern that has a characteristic peak in degrees 2θ at about 27.2. In yet another embodiment, crystalline salt Form 14-A is characterized by a powder X-ray diffraction pattern having at least one or more characteristic peaks in degrees 2θ at about 4.4, 6.1, 6.4, 6.7, 8.8, 9.5, 10.3, 15.3, 17.5, 21.3, 21.9, and 27.2. For example, a contemplated crystalline salt form has a powder X-ray diffraction pattern shown in FIG. 65. In one embodiment, the powder X-ray diffraction pattern of the crystalline form was obtained using Cu Kα radiation.

In another embodiment, a different crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, characterized by a powder X-ray diffraction pattern having a characteristic peak in degrees 2θ at about 7.7 (referred to herein as “Form 14-B”), is disclosed herein.

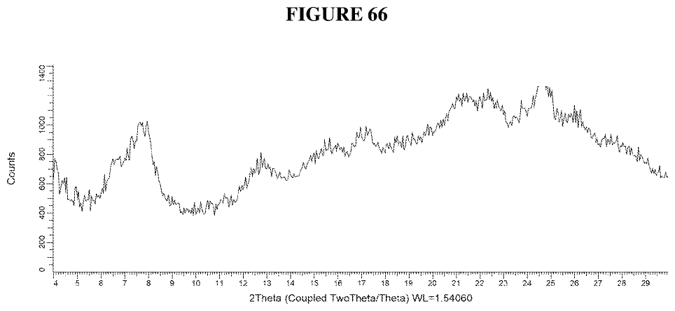

In one embodiment, the crystalline salt Form 14-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, is characterized by a powder X-ray diffraction pattern that has a characteristic peak in degrees 2θ at about 6.8, is characterized by a powder X-ray diffraction pattern that has a characteristic peak in degrees 2θ at about 12.9, is characterized by a powder X-ray diffraction pattern that has a characteristic peak in degrees 2θ at about 15.3, is characterized by a powder X-ray diffraction pattern that has a characteristic peak in degrees 2θ at about 17.2, is characterized by a powder X-ray diffraction pattern that has a characteristic peak in degrees 2θ at about 21.4, is characterized by a powder X-ray diffraction pattern that has a characteristic peak in degrees 2θ at about 22.4, is characterized by a powder X-ray diffraction pattern that has a characteristic peak in degrees 2θ at about 24.7, and/or is characterized by a powder X-ray diffraction pattern that has a characteristic peak in degrees 2θ at about 25.9. In yet another embodiment, crystalline salt Form 13-B is characterized by a powder X-ray diffraction pattern having at least one or more characteristic peaks in degrees 2θ at about 6.8, 7.7, 12.9, 15.3, 17.2, 21.4, 22.4, 24.7, and 25.9. For example, a contemplated crystalline salt form has a powder X-ray diffraction pattern shown in FIG. 66. In one embodiment, the powder X-ray diffraction pattern of the crystalline form was obtained using Cu Kα radiation.

In a further embodiment, a pharmaceutical composition comprising a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine and a pharmaceutically acceptable excipient is disclosed herein. For example, a pharmaceutical composition formed from a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein. In some embodiments, a disclosed pharmaceutical composition is a formulation for oral administration.

In an embodiment, a drug substance comprising at least a detectable amount of a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein. In another embodiment, a drug substance comprising a substantially pure crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine is disclosed herein.

### Compositions

Another aspect of the disclosure provides pharmaceutical compositions comprising crystalline compounds as disclosed herein formulated together with a pharmaceutically acceptable excipient. In particular, the present disclosure provides pharmaceutical compositions comprising crystalline compounds as disclosed herein formulated together with one or more pharmaceutically acceptable excipients. These formulations include those suitable for oral, topical (e.g., transdermal), buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic excipient or excipient suitable for external, enteral or parenteral applications. The active ingredient may be compounded, for example, with the usual non-toxic, pharmaceutically acceptable excipients for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical excipient, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable excipients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, nano-suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or excipients comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable excipient, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable excipients and stabilizers. The excipients and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

Amounts of a crystalline compound as described herein in a formulation may vary according to factors such as the disease state, age, sex, and weight of the individual. Dosage regimens can be adjusted to provide the optimum therapeutic response. For example, a single bolus can be administered, several divided doses may be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active crystalline compound calculated to produce the desired therapeutic effect in association with the required pharmaceutical carrier.

The specification for the dosage unit forms of the disclosure are dictated by and directly dependent on (a) the unique characteristics of the crystalline compound selected and the particular therapeutic effect to be achieved, and (b) the limitations inherent in the art of compounding such an active crystalline compound for the treatment of sensitivity in individuals.

Disclosed compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is suitable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

A disclosed crystalline compound can be administered in a time release formulation, for example in a composition which includes a slow release polymer. The crystalline compound can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

In accordance with an alternative aspect of the disclosure, a disclosed crystalline compound can be formulated with one or more additional compounds that enhance the solubility of the compound.

### Methods

In some embodiments, the disclosure provides a method of treating a disease or disorder associated with modulation of Embryonic Ectoderm Development (EED) in a patient in need thereof, comprising administering to the patient an effective amount of a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a disease or disorder associated with modulation of Embryonic Ectoderm Development (EED) in a patient in need thereof, comprising administering to the patient an effective amount of a pharmaceutical composition comprising a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In some embodiments, the disclosure provides a method of treating a disease or disorder associated with modulation of Polycomb Repressive Complex 2 (PRC2) in a patient in need thereof, comprising administering to the patient an effective amount of a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a disease or disorder associated with modulation of Polycomb Repressive Complex 2 (PRC2) in a patient in need thereof, comprising administering to the patient an effective amount of a pharmaceutical composition comprising a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In some embodiments, the disease or disorder may be a blood disorder. In certain embodiments, the disclosure provides a method of treating a blood disorder in a patient in need thereof, comprising administering to the patient an effective amount of a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a blood disorder in a patient in need thereof, comprising administering to the patient an effective amount of a pharmaceutical composition comprising a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In certain embodiments, the blood disorder may be selected from the group consisting of, for example, acute lymphoblastic leukemia (ALL), acute myeloid leukemia (ANIL), amyloidosis, anemia, aplastic anemia, bone marrow failure syndromes, chronic lymphocytic leukemia (CLL), chronic myeloid leukemia (CML), deep vein thrombosis (DVT), Diamond-Blackfan anemia, diffused large B cell lymphoma, dyskeratosis congenita (DKC), eosinophilic disorder, essential thrombocythemia, Fanconi anemia, follicular lymphoma, Gaucher disease, hemochromatosis, hemolytic anemia, hemophilia, hereditary spherocytosis, Hodgkin's lymphoma, idiopathic thrombocytopenic purpura (ITP), inherited bone marrow failure syndromes, iron-deficiency anemia, Langerhans ceil histiocytosis, large granular lymphocytic (LGL) leukemia, leukemia, leukopenia, mastocytosis, monoclonal gammopathy, multiple myeloma, myelodysplastic syndromes (MDS), myelofibrosis, myeloproliferative neoplasms (MPN), non-Hodgkin's lymphoma, paroxysmal nocturnal hemoglobinuria (PNH), pernicious anemia (B12 deficiency), polycythemia vera, porphyria, post-transplant lymphoproliferative disorder (PTLD), pulmonary embolism (PE), Shwachman-Diamond syndrome (SDS), sickle cell disease (SCD), β-thalassemia, thrombocytopenia, thrombotic thrombocytopenic purpura (TTP), venous thromboembolism, Von Willebrand disease, and Valdenstrom's macroglobulinemia (lymphoplasmacytic lymphoma). In some embodiments, the blood disorder is sickle cell disease (SCD). In other embodiments, the blood disorder is β-thalassemia.

In some embodiments, the disease or disorder may be a cancer. In certain embodiments, the disclosure provides a method of treating a cancer in a patient in need thereof, comprising administering to the patient an effective amount of a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine. In other embodiments, the disclosure provides a method of treating a cancer in a patient in need thereof, comprising administering to the patient an effective amount of a pharmaceutical composition comprising a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In certain embodiments, the cancer may be selected from the group consisting of, for example, mesothelioma, gastric cancer, malignant rhabdoid tumor, hepatocellular carcinoma, prostate cancer, breast carcinoma, bile duct and gallbladder cancers, bladder carcinoma, brain tumors including neuroblastoma, Schwannoma, glioma, glioblastoma and astrocytoma, cervical cancer, colon cancer, melanoma, endometrial cancer, esophageal cancer, head and neck cancer, lung cancer, nasopharyngeal carcinoma, ovarian cancer, pancreatic cancer, renal cell carcinoma, rectal cancer, thyroid cancers, parathyroid tumors, uterine tumors, and soft tissue sarcomas.

In other embodiments, the disclosure provides a method of treating thoracic aortic aneurysm, coronary heart disease, stenotic disease, pulmonary artery hypertension (PAR), liver fibrosis, allergic inflammation, retinitis pigmentosa, septic shock, herpes simplex virus, human cytomegalovirus, α-thalassemia, familial atrial fibrillation, common variable immunodeficiency, aneurysm-osteoarthritis syndrome, and acquired immunodeficiency syndrome in a patient in need thereof, comprising administering to the patient an effective amount of a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In other embodiments, the disclosure provides a method of treating thoracic aortic aneurysm, coronary heart disease, stenotic disease, pulmonary artery hypertension (PAR), liver fibrosis, allergic inflammation, retinitis pigmentosa, septic shock, herpes simplex virus, human cytomegalovirus, α-thalassemia, familial atrial fibrillation, common variable immunodeficiency, aneurysm-osteoarthritis syndrome, and acquired immunodeficiency syndrome in a patient in need thereof, comprising administering to the patient an effective amount of a pharmaceutical composition comprising a disclosed crystalline compound, for example, a disclosed crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, for example, a disclosed crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine.

In particular, in certain embodiments, the disclosure provides a method of treating the above medical indications comprising administering to a patient in need thereof an effective amount of a crystalline compound disclosed herein. In certain other embodiments, the disclosure provides a method of treating the above medical conditions in a patient in need thereof, comprising orally, subcutaneously, or intravenously administering to the patient a composition comprising a disclosed crystalline form.

The crystalline compounds disclosed herein can be used as a medicament or pharmaceutically acceptable composition, e.g., in the form of pharmaceutical preparations for oral, enteral, parenteral, or topical administration, and the contemplated methods disclosed herein may include administering orally, enterally, parenterally, or topically a disclosed crystalline compound, or a composition comprising or formed from such a disclosed crystalline compound. For example, a disclosed crystalline form may be capable of controlling one or more pharmacokinetic properties (e.g., a longer or shorter release profile) when administered by a certain route (e.g., oral) or in a certain formulation, as compared to a different route (e.g., subcutaneous) or other formulation e.g., a formulation having the amorphous form. In one embodiment, a disclosed crystalline form may afford substantial reproducibility from one formulation to another.

Also disclosed herein are pharmaceutical compositions comprising a disclosed crystalline compound and at least one additional therapeutic agent. In some embodiments, the additional therapeutic agent may be selected from the group consisting of, for example, anti-cancer agents, immunomodulators, anti-allergic agents, anti-emetics, pain relievers, cytoprotective agents, anti-sickling agents, and combinations thereof. In other embodiments, the additional therapeutic agent may be, for example, an EZH2 inhibitor. For example, in certain embodiments the additional therapeutic agent may be selected from the group consisting of N-((4,6-dimethyl-2-oxo-1,2-dihydropyridin-3-yl)methyl)-5-(ethyl(tetrahydro-2H-pyran-4-yl)amino)-4-methyl-4′-(morpholinomethyl)-[1,1′-biphenyl]-3-carboxamide (tazemetostat), (2R)-7-chloro-2-[4-(dirnethylamino)cyclohexyl]-N-[(4,6-dimethyl-2-oxo-1H-pyridin-3-yl)methyl]-2,4-dimethyl-1,3-benzodioxole-5-carboxamide (valemetostat, DS-3201b), N-[(4-methoxy-6-methyl-2-oxo-1H-pyridin-3-yl)methyl]-2-methyl-1 [(1R)-1-[1-(2,2,2-trifluoroethyl)piperidin-4-yl]ethyl]indole-3-carboxamide (CPI-1205), (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide (GSK28 1 6126), (R)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one (PF-06821497), SHR2554, and combinations thereof.

In further embodiments, the additional therapeutic agent may be, e.g., hydroxyurea. In certain embodiments, the additional therapeutic agent may be selected from the group consisting of, for example, 2-hydroxy-6-((2-(1-isopropyl-1H-pyrazol-5-yl)pyridin-3-yl)methoxy)benzaldehyde (voxelotor, GBT-440), P-Selectin antibodies, L-Glutamine, and combinations thereof.

In some embodiments, the additional therapeutic agent may be, for example, an anti-adhesion agent. For example, in certain embodiments the additional therapeutic agent may be selected from the group consisting of crizanlizumab (SEG101), (2S)-2-[(2R,3R,4S,5S,6R)-3-benzoyloxy-2-[(1R,2R,3S,5R)-3-[(2,4-dioxo-1H-pyrimidine-6-carbonyl)amino]-5-[2-[[2-[2-[2-oxo-2-[(3,6,8-trisulfonaphthalen-1-yl)amino]ethoxy]ethoxy]acetyl]amino]ethylcarbamoyl]-2-[(2S,3S,4R,5S,6S)-3,4,5-trihydroxy-6-methyloxan-2-yl]oxycyclohexyl]oxy-5-hydroxy-6-(hydroxymethyl)oxan-4-yl]oxy-3-cyclohexylpropanoic acid (rivipansel, GMI-1070), sevuparin, 6-[(3I,4S)-4-methy 1-1-(pyrimidin-2-ylmethyl)pyrrolidin-3-yl]-1-(oxan-4-yl)-5H-pyrazolo[3,4-d]pyrimidin-4-one (PF-04447943), inclacumab (LC1004-002), 3-[3-[4-(1-aminocyclobutyl)phenyl]-5-phenylimidazo[4,5-b]pyridin-2-yl]pyridin-2-amine (miransertib, ARQ 092), and combinations thereof.

In other embodiments, the additional therapeutic agent may be, for example, an anti-sickling agent. For example, in certain embodiments the additional therapeutic agent may be selected from the group consisting of 2-hydroxy-6-((2-(1-isopropyl-1H-pyrazol-5-yl)pyridin-3-yl)methoxy)benzaldehyde (voxelotor, GBT-440), 6-[(3S,4S)-4-methyl-1-(2-pyrimidinylmethyl)-3-pyrrolidinyl]-3-(tetrahydro-2H-pyran-4-yl)imidazo[1,5-a]pyrazin-8(7H)-one (IMR-687), and combinations thereof.

In further embodiments, the additional therapeutic agent may be, for example, a detoxification agent. For example, in certain embodiments the additional therapeutic agent may be LJPC-401. In some embodiments, the additional therapeutic agent may be selected from, for example, anti-inflammatory agents, anti-thrombotic agents, and combinations thereof. For example, in certain embodiments the additional therapeutic agent may be selected from the group consisting of (1S,2S,3R,5S)-3-[7-{[(1R,2S)-2-(3,4-difluorophenyl)cyclopropyl]amino}-5(propylthio)-3H-[1,2,3]-triazolo[4,5-d]pyrimidin-3-yl]-5-(2-hydroxyethoxy)cyclopentane-1,2-diol (brilinta, tricagrelor), (2R)-3,3,3-trifluoro-2-[[[5-fluoro-2-[1-[(2-fluorophenyl)methyl]-5-(1,2-oxazol-3-yl)pyrazol-3-yl]pyrimidin-4-yl]amino]methyl]-2-hydroxypropanamide (olinciguat), NKTT120, and combinations thereof.

In some embodiments, the additional therapeutic agent may be, for example, sanguinate. In other embodiments, the additional therapeutic agent may be, for example, an agent that causes disruption of PRC2. In further embodiments, the additional therapeutic agent is, for example, AZD9291.

### EXAMPLES

The compounds described herein can be prepared in a number of ways based on the teachings contained herein and synthetic procedures known in the art. The following non-limiting examples illustrate the disclosure.

X-ray powder diffraction was performed using a Bruker D8 Advance equipped with Lynxeye detector in reflection mode (Bragg-Brentano geometry). Samples were prepared on Si zero-return wafers. The parameters for XRPD methods used are listed in Table 1 below.

TABLE 1

| Parameter | Regular Scan |
|---|---|
| X-ray wavelength | Cu Kα1, 1.540598 Å |
| X-ray tube setting | 40 kV, 40 mA |
| Slit condition | 0.6 mm div. + 2.5° soller |
| Scan mode | Step |
| Scan range (°2θ) | 4-30 |
| Step size (°2θ) | 0.03 |
| Dwell time (s/step) | 0.23 |
| Spin | Yes (0.5 Hz) |

X-ray powder diffraction was also performed using a Rigaku MiniFlex 600 in reflection mode (Bragg-Brentano geometry). Samples were prepared on Si zero-return wafers. The parameters for XRPD methods used are listed in Table 2 below.

TABLE 2

| Parameter | Regular Scan |
|---|---|
| X-ray wavelength | Cu Kα1, 1.540598 Å |
| X-ray tube setting | 40 kV, 15 mA |
| Slit condition | 1.25° div., Ni kβ filter, 0.3 mm rec. |
| Scan mode | Continuous |
| Scan range (°2θ) | 4-30 |
| Step size (°2θ) | 0.05 |
| Scan speed (°/min) | 5 |
| Spin | No |

Differential scanning calorimetry (DSC) and thermogravimetric analysis (TGA) were performed using a Mettler Toledo DSC³⁺. Samples (3-5 mg) were weighed directly in a hermetic aluminum pan with pinhole and analyzed according to the parameters in Table 3A below.

TABLE 3A
Parameters

| | |
|---|---|
| Method | Ramp |
| Sample size | 3-5 mg |
| Heating rate | 10.0° C./min |
| Temperature range | 30 to 300° C. |
| Heating gas | N₂ at 60.00 mL/min |

Dynamic vapor sorption (DVS) analysis was performed using a DVS Intrinsic 1. The sample (25 mg) was loaded into a sample pan, suspended from a microbalance and exposed to a humidified stream of nitrogen gas. The sample was held for a minimum of 5 min at each level and only progressed to the next humidity level if there was <0.002% change in weight between measurements (interval: 60 seconds) or 240 min had elapsed. The following program was used as shown in Table 3B:

TABLE 3B

| | |
|---|---|
| 1) | Equilibration at 50% RH |
| 2) | 50% to 2% (50%, 40%, 30%, 20%, 10% and 2%) |
| 3) | 2% to 95% (2%, 10%, 20%, 30%, |
| | 40%, 50%, 60%, 70%, 80%, 90%, 95%) |
| 4) | 95% to 2% (95%, 80%, 70%, 60%, 50%, |
| | 40%, 30%, 20%, 10%, 2%) |
| 5) | 2% to 50% (2%, 10%, 20%, 30%, 40%, 50%) |

Optical microscopy was performed using a Zeiss AxioScope A1 equipped with 2.5×, 10×, 20× and 40× objectives and polarizer. Images are captured through a built-in Axiocam 105 digital camera and processed using ZEN 2 (blue edition) software provided by Zeiss.

### Example 1

Crystalline, Form P material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 25 mg of Form E material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was dissolved in acetone (50 volumes) at 50° C. The solution was transferred at once to a solution of heptane (400 volumes) at 50° C. with rapid stirring until formation of a white slurry. The solids were collected at 50° C. and dried overnight. XRPD analysis indicated that the material was crystalline with a pattern consistent with Form P.

Crystalline, Form P material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was also prepared as follows. About 25 mg of Form E material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was dissolved in isopropyl alcohol (25 volumes) at 50° C. The solution was transferred at once to a solution of water (100 volumes) at 50° C. with rapid stirring until formation of a white slurry. The solids were collected at 50° C. and dried overnight. XRPD analysis indicated that the material was crystalline with a pattern consistent with Form P.

The XRPD pattern of Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 1. Characteristic peaks include one or more of the peaks shown in Table 4.

TABLE 4

| | | Relative |
|---|---|---|
| | d-spacing | intensity |
| 2θ (deg) | (Å) | (a.u) |
| 7.65 | 11.54 | 100 |
| 8.64 | 10.23 | 3 |
| 11.86 | 7.45 | 40 |
| 13.90 | 6.37 | 6 |
| 14.45 | 6.12 | 20 |
| 15.33 | 5.78 | 60 |
| 16.15 | 5.48 | 12 |
| 17.19 | 5.16 | 8 |
| 17.30 | 5.12 | 12 |
| 20.75 | 4.28 | 23 |
| 22.58 | 3.93 | 13 |
| 23.28 | 3.82 | 8 |
| 24.53 | 3.63 | 7 |
| 26.18 | 3.40 | 9 |
| 27.99 | 3.19 | 3 |
| 28.18 | 3.16 | 5 |
| 29.60 | 3.02 | 5 |

FIG. 2 depicts the differential scanning calorimetry (DSC) profile of crystalline Form P. As shown in FIG. 2, crystalline Form P shows a characteristic endotherm with an onset of about 252° C. and a peak of about 253° C.

Crystalline Form P of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base, displayed a thermogravimetric analysis (TGA) profile showing a mass loss of about 0.46 wt. % up to about 260° C. Crystalline Form P displayed a dynamic vapor sorption (DVS) profile showing a reversable total mass change of about 0.53 wt. % between about 2 to about 92% relative humidity (RH) at 25° C. Crystalline Form P displayed a rod-like and/or a plate-like morphology by optical microscopy. Crystalline Form P was shown to be stable for at least one week under drying and high humidity conditions (40° C. and 75% relative humidity). Form P maintained crystallinity and solid form purity through a wet-milling study (ball mill, 30 s, milled with 1 vol. water).

### Example 2

Crystalline, Form A material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. NH₄OH (14.5 M) was added dropwise to a methanol solution of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, at room temperature. After further addition of 1 mL NH₄OH, the solution became an immobile gel. An additional 2 volumes of MeOH was added to the gel, mixed manually, heated to 50° C. and stirred overnight. The following morning, opaque white fibrous particles were observed among the gel. The mixture was sonicated to generate a flowable, off-white slurry. XRPD analysis of a drawn sample indicated that the material was crystalline with a pattern consistent with Form A.

The XRPD pattern of Form A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 3. Characteristic peaks include one or more of the peaks shown in Table 5.

TABLE 5

| | | Relative |
|---|---|---|
| | d-spacing | intensity |
| 2θ (deg) | (Å) | (a.u) |
| 5.90 | 14.96 | 39 |
| 7.11 | 12.42 | 5 |
| 8.52 | 10.37 | 100 |
| 9.33 | 9.48 | 55 |
| 9.48 | 9.32 | 35 |
| 9.96 | 8.87 | 19 |
| 10.74 | 8.23 | 33 |
| 11.73 | 7.54 | 62 |
| 13.24 | 6.68 | 11 |
| 14.13 | 6.26 | 12 |
| 14.43 | 6.13 | 28 |
| 14.98 | 5.91 | 8 |
| 15.21 | 5.82 | 10 |
| 15.72 | 5.63 | 9 |
| 15.88 | 5.58 | 9 |
| 18.73 | 4.73 | 19 |
| 19.03 | 4.66 | 38 |
| 20.04 | 4.43 | 12 |
| 20.68 | 4.29 | 10 |
| 21.72 | 4.09 | 10 |
| 24.58 | 3.62 | 13 |
| 25.80 | 3.45 | 19 |
| 27.08 | 3.29 | 7 |
| 28.40 | 3.14 | 17 |

### Example 3

Crystalline, Form B material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. NH₄OH (14.5 M) was added dropwise to a methanol solution of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, at 50° C. After further addition of 1 mL NH₄OH, the solution became an immobile gel. An additional 6 volumes of MeOH was added to the gel, mixed manually, and stirred overnight. The following morning, opaque white fibrous particles were observed among the gel. The mixture was sonicated and generated a nice flowable off-white slurry. A sample was drawn for XRPD analysis and showed a crystalline form, Form B. The slurry was stirred for an additional 5 hours at room temperature. The solids were collected by filtration and rinsed with 4 volumes of MeOH:water (1:3 vol.). The wet-cake was pressed to an XRPD plate and the sample analyzed. XRPD analysis of a drawn sample indicated that the material was crystalline with a pattern consistent with Form B. Crystalline Form P displayed a hair-like morphology by optical microscopy.

The XRPD pattern of Form B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 4. Characteristic peaks include one or more of the peaks shown in Table 6.

TABLE 6

| | | Relative |
|---|---|---|
| | d-spacing | intensity |
| 2θ (deg) | (Å) | (a.u) |
| 5.10 | 17.30 | 17 |
| 5.93 | 14.88 | 18 |
| 8.47 | 10.44 | 100 |
| 9.41 | 9.39 | 54 |
| 10.02 | 8.82 | 30 |
| 10.75 | 8.22 | 52 |
| 11.48 | 7.70 | 37 |
| 11.69 | 7.56 | 28 |
| 13.07 | 6.77 | 6 |
| 14.13 | 6.26 | 12 |
| 14.45 | 6.13 | 23 |
| 15.37 | 5.76 | 11 |
| 15.66 | 5.66 | 17 |
| 16.49 | 5.37 | 8 |
| 18.58 | 4.77 | 28 |
| 19.01 | 4.67 | 16 |
| 19.76 | 4.49 | 31 |
| 20.62 | 4.30 | 25 |
| 21.42 | 4.14 | 17 |
| 22.34 | 3.98 | 5 |
| 25.64 | 3.47 | 16 |
| 26.55 | 3.35 | 14 |
| 28.35 | 3.15 | 15 |
| 29.37 | 3.04 | 13 |

### Example 4

Crystalline, Form C material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. The wet cake Form B material obtained in Example 3 above was dried in a vacuum oven equipped with a rotary oil vacuum pump overnight at 50° C. XRPD analysis of the dried sample indicated that the material was crystalline with a pattern consistent with Form C.

The XRPD pattern of Form C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 5. Characteristic peaks include one or more of the peaks shown in Table 7.

TABLE 7

| | | Relative |
|---|---|---|
| | d-spacing | intensity |
| 2θ (deg) | (Å) | (a.u) |
| 4.02 | 21.94 | 53 |
| 5.34 | 16.54 | 24 |
| 5.73 | 15.42 | 6 |
| 6.34 | 13.94 | 5 |
| 7.58 | 11.66 | 23 |
| 8.99 | 9.83 | 100 |
| 9.69 | 9.12 | 38 |
| 11.18 | 7.91 | 68 |
| 12.32 | 7.18 | 47 |
| 12.88 | 6.87 | 76 |
| 13.73 | 6.44 | 40 |
| 14.46 | 6.12 | 77 |
| 15.31 | 5.78 | 13 |
| 15.89 | 5.57 | 41 |
| 19.04 | 4.66 | 28 |

### Example 5

Crystalline, Form D material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. 51.6 mg of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, was dissolved in 18 volumes of MeOH at 50° C. Half of the determined amount of NH₄OH required for precipitation was added, followed by seeding with Form B, resulting in immediate precipitation of fluffy solids. Addition of the second half of the NH₄OH was completed and the slurry stirred for 2 hours at 50° C. The slurry was allowed to cool to RT and stirred for 1.25 hours. The solids were collected by filtration, rinsed three times with 1 mL water, and dried in a vacuum oven equipped with a rotary oil vacuum pump at 50° C. for 3 days. XRPD analysis of the dried solid indicated that the material was crystalline with a pattern consistent with Form D.

The XRPD pattern of Form D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 6. Characteristic peaks include one or more of the peaks shown in Table 8.

TABLE 8

| | | Relative |
|---|---|---|
| | d-spacing | intensity |
| 2θ (deg) | (Å) | (a.u) |
| 7.33 | 12.05 | 32 |
| 8.64 | 10.23 | 100 |
| 10.59 | 8.34 | 13 |
| 12.93 | 6.84 | 44 |

### Example 6

Crystalline, Form E material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, was prepared as follows. 33 mg of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, was added to a vial, followed by 2 mL (˜60 volumes) of saturated NaHCO₃ (aq.) solution. The slurry was stirred at 50° C. for 1 hour. The slurry solids were collected by filtration and the wet cake analyzed by XRPD. XRPD analysis indicated that the wet cake material was crystalline with a pattern consistent with Form E.

The XRPD pattern of Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, is shown in FIG. 7. Characteristic peaks include one or more of the peaks shown in Table 9.

TABLE 9

| | | Relative |
|---|---|---|
| | d-spacing | intensity |
| 2θ (deg) | (Å) | (a.u) |
| 4.59 | 19.23 | 100 |
| 4.97 | 17.77 | 5 |
| 6.70 | 13.19 | 19 |
| 8.61 | 10.27 | 21 |
| 9.32 | 9.48 | 61 |
| 11.58 | 7.64 | 42 |
| 12.96 | 6.82 | 5 |
| 13.25 | 6.67 | 26 |
| 13.46 | 6.57 | 31 |
| 13.66 | 6.48 | 39 |
| 14.60 | 6.06 | 7 |
| 15.10 | 5.86 | 28 |
| 16.59 | 5.34 | 7 |
| 18.08 | 4.90 | 27 |
| 18.51 | 4.79 | 64 |
| 19.35 | 4.58 | 29 |
| 20.40 | 4.35 | 22 |
| 21.68 | 4.10 | 10 |
| 22.02 | 4.03 | 5 |
| 22.40 | 3.97 | 20 |
| 25.72 | 3.46 | 42 |
| 27.02 | 3.30 | 53 |
| 27.40 | 3.25 | 9 |
| 27.90 | 3.19 | 17 |
| 29.35 | 3.04 | 16 |

FIG. 8 depicts the differential scanning calorimetry (DSC) profile of crystalline Form E. As shown in FIG. 8, crystalline Form E shows a characteristic endotherm with an onset of about 44° C. and a peak of about 58° C., a characteristic endotherm with an onset of about 110° C. and a peak of about 114° C., and a characteristic endotherm with an onset of about 166° C. and a peak of about 177° C.

Crystalline Form E of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, displayed a thermogravimetric analysis (TGA) profile showing a mass loss of about 5.1 wt. % up to about 170° C. Crystalline Form E displayed a dynamic vapor sorption (DVS) profile showing a reversable total mass change of about 10.3 wt. % between about 2 to about 92% relative humidity (RH) at 25° C. Crystalline Form E displayed a hair-like morphology by optical microscopy. Crystalline Form E was shown to be stable for at least one week at 40° C. and 75% relative humidity (RH).

### Example 7

Crystalline, Form F material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A sample of crystalline, Form E material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, was further dried at 50° C. overnight in a vacuum oven equipped with a rotary oil vacuum pump. XRPD analysis indicated that the material was crystalline with a pattern consistent with Form F.

The XRPD pattern of Form F of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 9. Characteristic peaks include one or more of the peaks shown in Table 10.

TABLE 10

| | | Relative |
|---|---|---|
| | d-spacing | intensity |
| 2θ (deg) | (Å) | (a.u) |
| 4.55 | 19.41 | 100 |
| 6.96 | 12.70 | 10 |
| 8.21 | 10.77 | 12 |
| 8.97 | 9.85 | 16 |
| 9.33 | 9.47 | 64 |
| 12.51 | 7.07 | 15 |
| 13.55 | 6.53 | 91 |
| 15.29 | 5.79 | 5 |
| 17.76 | 4.99 | 17 |
| 18.75 | 4.73 | 52 |
| 22.62 | 3.93 | 26 |
| 26.84 | 3.32 | 9 |

### Example 8

Crystalline, Form H material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, was prepared as follows. A slurry of Form E material in MeOH was stirred at 50° C. for 3 days. The slurry solids were collected by filtration and dried in a vacuum oven at 50° C. for 16 hours. XRPD analysis indicated that the material was crystalline with a pattern consistent with Form H.

Crystalline, Form H material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, was also prepared as follows. Ten volumes MeOH was added to 0.21 g of Form P material that had been dried in a 20 mL vial. The resultant slurry was seeded with Form H material. After 2 hours, the slurry was sampled and showed an XRPD pattern consistent with crystalline Pattern H. After stirring the slurry overnight, an additional 10 volumes of MeOH was added, and the slurry was transferred to a 4 mL vial to continue stirring. 15 μL of water was added. The slurry was heated to 50° C., seeded again with Form H material, and stirred at 50° C. for 40 minutes. The slurry was cooled to room temperature, the solids were collected by filtration, rinsed once with 2 volumes of MeOH, then dried in a vacuum oven at 50° C. XRPD analysis indicated that the dried material was crystalline with a pattern consistent with Form H.

The XRPD pattern of Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, is shown in FIG. 10. Characteristic peaks include one or more of the peaks shown in Table 11.

TABLE 11

| | | Relative |
|---|---|---|
| | d-spacing | intensity |
| 2θ (deg) | (Å) | (a.u) |
| 5.28 | 16.73 | 51 |
| 7.18 | 12.31 | 5 |
| 8.48 | 10.42 | 100 |
| 10.44 | 8.47 | 94 |
| 12.38 | 7.14 | 23 |
| 13.28 | 6.66 | 39 |
| 17.01 | 5.21 | 11 |
| 19.48 | 4.55 | 9 |
| 22.62 | 3.93 | 7 |
| 24.54 | 3.62 | 6 |
| 26.22 | 3.40 | 5 |
| 26.69 | 3.34 | 5 |

FIG. 11 depicts the differential scanning calorimetry (DSC) profile of crystalline Form H. As shown in FIG. 11, crystalline Form H shows a characteristic endotherm with an onset of about 58° C. and a peak of about 84° C., a characteristic endotherm with an onset of about 63° C. and a peak of about 89° C., and a characteristic endotherm with an onset of about 169° C. and a peak of about 176° C.

Crystalline Form H of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate, displayed a thermogravimetric analysis (TGA) profile showing a mass loss of about 6.3 wt. % up to about 130° C. Crystalline Form H displayed a dynamic vapor sorption (DVS) profile showing a reversable total mass change of about 7.6 wt. % between about 2 to about 92% relative humidity (RH) at 25° C. Crystalline Form H displayed a hair-like morphology by optical microscopy. Crystalline Form H was shown to be stable for at least one week at 40° C. and 75% relative humidity (RH).

### Example 9

Crystalline, Form I material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A slurry of Form E material in EtOH was stirred at room temperature for 3 days. The slurry solid was filtered, and the wet cake analyzed by XRPD. XRPD analysis indicated that the wet cake material was crystalline with a pattern consistent with Form I.

Crystalline, Form I material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was also prepared as follows. Form E material was dissolved in EtOH (30 volumes) at 50° C. The stirred solution was cooled from 50° C. to room temperature at 5° C. per hour, accomplished by reducing the hot plate temperature by 2.5° C. every 30 minutes. The resulting slurry was further stirred for 3 days at room temperature, and the slurry solids were collected by filtration. XRPD analysis indicated that the material was crystalline with a pattern consistent with Form I. Crystalline Form I displayed a hair-like morphology by optical microscopy.

The XRPD pattern of Form I of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 12. Characteristic peaks include one or more of the peaks shown in Table 12.

TABLE 12

| | | Relative |
|---|---|---|
| | d-spacing | intensity |
| 2θ (deg) | (Å) | (a.u) |
| 5.23 | 16.88 | 22 |
| 7.07 | 12.50 | 18 |
| 8.37 | 10.55 | 100 |
| 10.37 | 8.52 | 87 |
| 12.22 | 7.24 | 12 |
| 13.06 | 6.78 | 42 |
| 16.80 | 5.27 | 17 |
| 19.24 | 4.61 | 5 |
| 20.67 | 4.29 | 7 |
| 22.30 | 3.98 | 7 |
| 25.91 | 3.44 | 7 |

### Example 10

Crystalline, Form J material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A slurry of Form E material in acetone was stirred at room temperature for 3 days. The slurry solids were filtered, and the wet cake analyzed by XRPD. XRPD analysis indicated that the wet cake material was crystalline with a pattern consistent with Form J.

Crystalline, Form J material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was also prepared as follows. Form E material was dissolved in acetone (34 volumes) at 50° C. The stirred solution was cooled from 50° C. to room temperature at 5° C. per hour, accomplished by reducing the hot plate temperature by 2.5° C. every 30 minutes. The resulting slurry was further stirred for 3 days at room temperature, and the slurry solids were collected by filtration. XRPD analysis indicated that the material was crystalline with a pattern consistent with Form J. Crystalline Form J displayed a hair-like morphology by optical microscopy.

The XRPD pattern of Form J of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 13. Characteristic peaks include one or more of the peaks shown in Table 13.

TABLE 13

| | | Relative |
|---|---|---|
| | d-spacing | intensity |
| 2θ (deg) | (Å) | (a.u) |
| 4.11 | 21.47 | 100 |
| 6.17 | 14.31 | 14 |
| 8.02 | 11.01 | 46 |
| 9.27 | 9.54 | 69 |
| 12.84 | 6.89 | 10 |
| 16.57 | 5.35 | 16 |
| 18.42 | 4.81 | 5 |
| 23.82 | 3.73 | 5 |
| 24.38 | 3.65 | 6 |
| 28.32 | 3.15 | 5 |

### Example 11

Crystalline, Form K material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A slurry of Form E material in MeCN was stirred at room temperature for 3 days. The slurry solids were filtered, and the wet cake analyzed by XRPD. XRPD analysis indicated that the wet cake material was crystalline with a pattern consistent with Form K.

Crystalline, Form K material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was also prepared as follows. Form E material was dissolved in MeCN (60 volumes) at 50° C. The stirred solution was cooled from 50° C. to room temperature at 5° C. per hour, accomplished by reducing the hot plate temperature by 2.5° C. every 30 minutes. The resulting slurry was further stirred for 3 days at room temperature, and the slurry solids were collected by filtration. XRPD analysis indicated that the material was crystalline with a pattern consistent with Form K.

The XRPD pattern of Form K of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 14. Characteristic peaks include one or more of the peaks shown in Table 14.

TABLE 14

| | | Relative |
|---|---|---|
| | d-spacing | intensity |
| 2θ (deg) | (Å) | (a.u) |
| 6.39 | 13.83 | 25 |
| 9.63 | 9.18 | 100 |
| 10.53 | 8.40 | 5 |
| 13.62 | 6.50 | 65 |
| 19.37 | 4.58 | 11 |
| 20.99 | 4.23 | 7 |
| 22.36 | 3.97 | 9 |
| 23.36 | 3.81 | 5 |
| 23.71 | 3.75 | 8 |
| 25.52 | 3.49 | 10 |
| 26.90 | 3.31 | 7 |

FIG. 15 depicts the differential scanning calorimetry (DSC) profile of crystalline Form K. As shown in FIG. 15, crystalline Form K shows a characteristic endotherm with an onset of about 226° C. and a peak of about 230° C.

### Example 12

Crystalline, Form L material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A slurry of Form E material in MeCN was stirred at 50° C. for 3 days. The slurry solids were filtered, and the wet cake analyzed by XRPD. XRPD analysis indicated that the wet cake material was crystalline with a pattern consistent with Form L.

The XRPD pattern of Form L of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 16. Characteristic peaks include one or more of the peaks shown in Table 15.

TABLE 15

| | | Relative |
|---|---|---|
| | d-spacing | intensity |
| 2θ (deg) | (Å) | (a.u) |
| 6.74 | 13.10 | 20 |
| 8.35 | 10.58 | 100 |
| 9.08 | 9.73 | 52 |
| 9.46 | 9.34 | 36 |
| 11.90 | 7.43 | 19 |
| 12.24 | 7.23 | 28 |
| 14.30 | 6.19 | 42 |
| 16.21 | 5.46 | 14 |
| 16.62 | 5.33 | 6 |
| 20.49 | 4.33 | 40 |
| 21.90 | 4.06 | 19 |
| 23.15 | 3.84 | 12 |
| 25.52 | 3.49 | 29 |
| 27.29 | 3.27 | 10 |
| 28.59 | 3.12 | 9 |

### Example 13

Crystalline, Form M material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. The wet cake material of Form L, obtained from Example 12, was further dried in a vacuum oven at 50° C. for 16 hours. XRPD analysis indicated that the dry material was crystalline with a pattern consistent with Form M.

The XRPD pattern of Form M of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 17. Characteristic peaks include one or more of the peaks shown in Table 16.

TABLE 16

| | | Relative |
|---|---|---|
| | d-spacing | intensity |
| 2θ (deg) | (Å) | (a.u) |
| 6.43 | 13.74 | 30 |
| 6.81 | 12.97 | 14 |
| 8.32 | 10.62 | 45 |
| 9.63 | 9.18 | 100 |
| 11.91 | 7.43 | 7 |
| 12.71 | 6.96 | 11 |
| 13.40 | 6.60 | 30 |
| 14.29 | 6.19 | 21 |
| 16.62 | 5.33 | 7 |
| 17.29 | 5.12 | 13 |
| 19.57 | 4.53 | 16 |
| 20.98 | 4.23 | 21 |
| 23.94 | 3.71 | 8 |
| 26.49 | 3.36 | 12 |

### Example 14

Crystalline, Form N material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. Form E material was dissolved in MeCN:water (85:15, 12 volumes) at 50° C. The stirred solution was cooled from 50° C. to room temperature at 5° C. per hour, accomplished by reducing the hot plate temperature by 2.5° C. every 30 minutes. The resulting slurry was further stirred for 3 days at room temperature. The slurry solids were filtered, and the wet cake analyzed by XRPD. XRPD analysis indicated that the wet cake material was crystalline with a pattern consistent with Form N.

The XRPD pattern of Form N of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 18. Characteristic peaks include one or more of the peaks shown in Table 17.

TABLE 17

| | | Relative |
|---|---|---|
| | d-spacing | intensity |
| 2θ (deg) | (Å) | (a.u) |
| 5.62 | 15.72 | 9 |
| 6.10 | 14.47 | 23 |
| 6.92 | 12.75 | 20 |
| 7.25 | 12.18 | 23 |
| 8.41 | 10.50 | 100 |
| 11.79 | 7.50 | 10 |
| 13.67 | 6.47 | 5 |
| 14.50 | 6.10 | 16 |
| 17.33 | 5.11 | 7 |
| 19.78 | 4.48 | 5 |
| 21.93 | 4.05 | 16 |
| 24.95 | 3.57 | 5 |
| 25.66 | 3.47 | 7 |

### Example 15

Crystalline, Form O material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 25 mg of Form E material was dissolved in DMSO (5 volumes) at room temperature. Water (10 volumes) was added to the stirred DMSO solution dropwise in 4 portions over 60 minutes. The resulting slurry solids were collected by filtration and dried overnight. XRPD analysis indicated that the material was crystalline with a pattern consistent with Form O.

The XRPD pattern of Form O of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 19. Characteristic peaks include one or more of the peaks shown in Table 18.

TABLE 18

| | | Relative |
|---|---|---|
| | d-spacing | intensity |
| 2θ (deg) | (Å) | (a.u) |
| 8.96 | 9.87 | 78 |
| 10.58 | 8.36 | 5 |
| 11.29 | 7.83 | 19 |
| 14.06 | 6.30 | 100 |
| 15.83 | 5.59 | 6 |
| 16.30 | 5.43 | 6 |
| 17.64 | 5.02 | 5 |
| 19.48 | 4.55 | 7 |
| 21.33 | 4.16 | 9 |
| 22.66 | 3.92 | 5 |
| 22.88 | 3.88 | 8 |
| 25.08 | 3.55 | 9 |
| 28.75 | 3.10 | 5 |

FIG. 20 depicts the differential scanning calorimetry (DSC) profile of crystalline Form O. As shown in FIG. 20, crystalline Form O shows a characteristic endotherm with an onset of about 166° C. and a peak of about 172° C., and a characteristic endotherm with an onset of about 196° C. and a peak of about 204° C.

Crystalline Form O of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, displayed a thermogravimetric analysis (TGA) profile showing a first step mass loss of about 0.23 wt. % up to about 150° C., a second step mass loss of about 1.7 wt. % between about 150° C. to about 200° C., a third step mass loss of about 1.3 wt. % between about 200° C. to about 250° C., and a fourth step mass loss of about 4.7 wt. % between about 250° C. to about 280° C.

### Example 16

Crystalline, Form Q material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 28 mg of Form E was loaded into a milling capsule and 1 volume of MeCN was added, along with a ¼″ steel ball as milling media. The solid was milled with a Wig-L-bug at 3500 rpm for 30 seconds, then collected and analyzed by XRPD. XRPD analysis of the wet cake material indicated that the material was crystalline with a pattern consistent with Form Q.

The XRPD pattern of Form Q of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 21. Characteristic peaks include one or more of the peaks shown in Table 19.

TABLE 19

| | | Relative |
|---|---|---|
| | d-spacing | intensity |
| 2θ (deg) | (Å) | (a.u) |
| 4.79 | 18.45 | 81 |
| 5.79 | 15.25 | 26 |
| 7.73 | 11.43 | 100 |
| 9.58 | 9.23 | 93 |
| 11.61 | 7.62 | 73 |
| 11.94 | 7.41 | 11 |
| 13.83 | 6.40 | 31 |
| 14.68 | 6.03 | 14 |
| 15.15 | 5.84 | 12 |
| 15.47 | 5.72 | 7 |
| 17.49 | 5.07 | 9 |
| 19.11 | 4.64 | 11 |
| 19.90 | 4.46 | 48 |
| 21.12 | 4.20 | 6 |
| 21.51 | 4.13 | 29 |
| 22.78 | 3.90 | 13 |
| 23.84 | 3.73 | 14 |
| 24.13 | 3.69 | 35 |
| 24.71 | 3.60 | 18 |
| 26.23 | 3.40 | 5 |
| 27.44 | 3.25 | 17 |
| 28.79 | 3.10 | 8 |

### Example 17

Crystalline, Form R material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. Form E material was dissolved in N,N-dimethylacetamide (9 volumes) at 50° C. The stirred solution was cooled from 50° C. to room temperature at 5° C. per hour, accomplished by reducing the hot plate temperature by 2.5° C. every 30 minutes. The resulting slurry was further stirred for 3 days at room temperature. The slurry solids were filtered and dried under vacuum overnight at 50° C. XRPD analysis indicated that the material was crystalline with a pattern consistent with Form R.

The XRPD pattern of Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 22. Characteristic peaks include one or more of the peaks shown in Table 20.

TABLE 20

| | | Relative |
|---|---|---|
| | d-spacing | intensity |
| 2θ (deg) | (Å) | (a.u) |
| 7.49 | 11.79 | 28 |
| 8.46 | 10.44 | 100 |
| 9.73 | 9.09 | 3 |
| 10.11 | 8.75 | 6 |
| 12.81 | 6.90 | 5 |
| 14.79 | 5.98 | 40 |
| 15.59 | 5.68 | 19 |
| 16.91 | 5.24 | 8 |
| 17.43 | 5.08 | 15 |
| 18.29 | 4.85 | 14 |
| 19.50 | 4.55 | 6 |
| 20.13 | 4.41 | 9 |
| 21.04 | 4.22 | 4 |
| 21.79 | 4.07 | 14 |

FIG. 23 depicts the differential scanning calorimetry (DSC) profile of crystalline Form R. As shown in FIG. 23, crystalline Form R shows a characteristic endotherm with an onset of about 148° C. and a peak of about 152° C., and a characteristic endotherm with an onset of about 241° C. and a peak of about 251° C.

The contemplated crystalline Form R of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, displayed a thermogravimetric analysis (TGA) profile showing a first step mass loss of about 0.87 wt. % up to about 150° C., a second step mass loss of about 3.1 wt. % between about 150° C. to about 200° C., and a third step mass loss of about 11.7 wt. % between about 200° C. to about 240° C.

### Example 18

Crystalline, Form S material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. The wet cake material of Form Q, obtained from Example 16, was further dried under vacuum. XRPD analysis indicated that the dry material was crystalline with a pattern consistent with Form S.

The XRPD pattern of Form S of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 24. Characteristic peaks include one or more of the peaks shown in Table 21.

TABLE 21

| | | Relative |
|---|---|---|
| | d-spacing | intensity |
| 2θ (deg) | (Å) | (a.u) |
| 4.98 | 17.72 | 100 |
| 5.95 | 14.85 | 84 |
| 7.99 | 11.06 | 48 |
| 9.67 | 9.14 | 47 |
| 11.78 | 7.51 | 25 |

### Example 19

Crystalline, Form T material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. A saturated solution of Form P was generated by making a slurry of Form P in isopropyl alcohol:water (1:4 vol.) and stirring overnight. The solids were allowed to settle, and the supernatant transferred into a vial containing about 5 mg each of Form E, Form H, and Form P. The competitive slurry was stirred at room temperature for 24 hours. XRPD analysis of the collected solids indicated that the material was crystalline with a pattern consistent with Form T.

The XRPD pattern of Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 25. Characteristic peaks include one or more of the peaks shown in Table 22.

TABLE 22

| | | Relative |
|---|---|---|
| | d-spacing | intensity |
| 2θ (deg) | (Å) | (a.u) |
| 7.00 | 12.61 | 13 |
| 8.73 | 10.12 | 100 |
| 10.32 | 8.57 | 9 |
| 11.78 | 7.51 | 7 |
| 14.08 | 6.28 | 4 |
| 17.04 | 5.20 | 14 |
| 20.92 | 4.24 | 9 |
| 22.42 | 3.96 | 5 |
| 24.49 | 3.63 | 7 |
| 25.52 | 3.49 | 3 |
| 27.88 | 3.20 | 4 |

FIG. 26 depicts the differential scanning calorimetry (DSC) profile of crystalline Form T. As shown in FIG. 26, crystalline Form T shows a characteristic endotherm with an onset of about 83° C. and a peak of about 84° C., and a characteristic endotherm with an onset of about 249° C. and a peak of about 251° C.

The contemplated crystalline Form T of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, displayed a thermogravimetric analysis (TGA) profile showing a first step mass loss of about 13.8 wt. % up to about 85° C. Crystalline Form T displayed a needle-like and/or a rod-like morphology by miscoscopy.

### Example 20

Crystalline, Form U material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. The wet cake material of Form N, obtained from Example 14, was further dried under vacuum overnight at 50° C. XRPD analysis indicated that the dry material was crystalline with a pattern consistent with Form U.

The XRPD pattern of Form U of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 27. Characteristic peaks include one or more of the peaks shown in Table 23.

TABLE 23

| | | Relative |
|---|---|---|
| | d-spacing | intensity |
| 2θ (deg) | (Å) | (a.u) |
| 6.20 | 14.63 | 100 |
| 6.93 | 12.75 | 51 |
| 7.58 | 11.65 | 40 |
| 9.01 | 9.81 | 62 |
| 10.49 | 8.42 | 24 |
| 12.29 | 7.19 | 37 |
| 14.55 | 6.08 | 24 |

### Example 21

Crystalline, Form V material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 12 mg of Form P material was loaded into a 2 mL vial. 2,2,2-Trifluoroethanol was added in aliquots at room temperature until dissolution (solubility 167-333 mg/mL). The solution was stirred overnight and the next day a slurry was observed. The slurry solids were collected and analyzed by XRPD. XRPD analysis indicated that the material was crystalline with a pattern consistent with Form V.

The XRPD pattern of Form V of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 28. Characteristic peaks include one or more of the peaks shown in Table 24.

TABLE 24

| | | Relative |
|---|---|---|
| | d-spacing | intensity |
| 2θ (deg) | (Å) | (a.u) |
| 6.04 | 14.63 | 31 |
| 7.98 | 11.08 | 49 |
| 10.44 | 8.47 | 17 |
| 10.84 | 8.15 | 19 |
| 12.03 | 7.35 | 24 |
| 13.12 | 6.74 | 6 |
| 13.80 | 6.41 | 5 |
| 15.65 | 5.66 | 40 |
| 15.91 | 5.57 | 35 |
| 16.76 | 5.29 | 35 |
| 18.30 | 4.84 | 100 |
| 18.78 | 4.72 | 10 |
| 19.74 | 4.49 | 23 |
| 20.89 | 4.25 | 23 |
| 21.02 | 4.22 | 17 |
| 22.77 | 3.90 | 7 |
| 23.55 | 3.77 | 7 |
| 23.94 | 3.71 | 7 |
| 24.68 | 3.60 | 5 |
| 25.80 | 3.45 | 9 |
| 26.33 | 3.38 | 28 |

### Example 22

Crystalline, Form W material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 2.5 mg of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt (Form 7-A), was loaded into a vial. 1 mL of water was added, and the mixture was warmed to 37° C. Additional water was added to the slurry, which thinned considerably and then appeared to start precipitating a fine white solid. This was viewed as likely disproportionation and additional water was not added. The mixture was stirred overnight, and the following day the fine solid suspended in the solution was collected and analyzed by XRPD. It was observed that the maleic acid salt had disproportionated over the slurry time. XRPD analysis indicated that a new crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, designated as Form W, was generated.

The XRPD pattern of Form W of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, is shown in FIG. 29. Characteristic peaks include one or more of the peaks shown in Table 25.

TABLE 25

| | | Relative |
|---|---|---|
| | d-spacing | intensity |
| 2θ (deg) | (Å) | (a.u) |
| 4.29 | 20.56 | 7 |
| 8.59 | 10.29 | 100 |
| 12.90 | 6.86 | 37 |
| 15.25 | 5.81 | 7 |
| 23.28 | 3.82 | 5 |
| 25.97 | 3.43 | 8 |

### Example 23

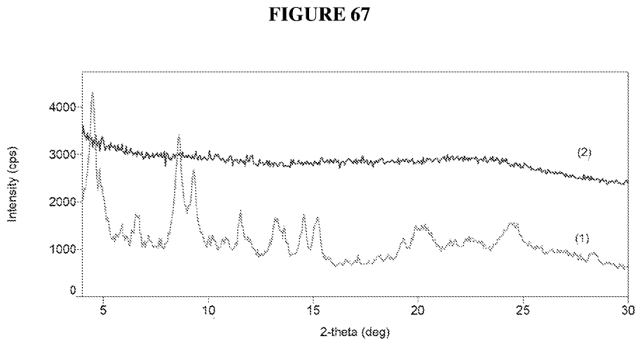

Amorphous form material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared as follows. About 30 mg of Form E material was dissolved in 5 volumes of THF at 50° C. and left uncapped without stirring for the solvent to evaporate. A yellow gel remained in the base of the vial and was dried in a vacuum oven overnight at 50° C. The brittle glass was broken up with a spatula and the solids were analyzed by XRPD to show an amorphous pattern (FIG. 67).

### Example 24—Salt Screening

A stock solution of Form P material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base, was prepared in 2,2,2-trifluoroethanol (50.8 mg/mL). Stock solutions of counter ion were prepared in EtOH.

Salt formation was carried out at room temperature in 2 mL vials. 25.4 mg of Form P free base material (500 μL stock solution) and 1.1 equivalents of counter ion were added to each vial. Solvent was allowed to evaporate at 40° C. overnight with stirring. The solvent in nearly half of the vials did not fully evaporate overnight. These vials were kept on the hotplate to continue evaporating (temperature increased to 50° C.), while the more-dry samples were dried further under vacuum for 3 hours at 50° C. The samples still containing solvent at the end of the day were left to stir at 30° C. over the weekend to finish evaporating the solvent. These samples were dried further under vacuum for 3 hours at 50° C.

Approximately 20 volumes of solvent (0.5 mL) was added to each vial containing the dried solids. The three solvents selected were EtOH, EtOAc, and IPA:water (9:1 vol). Once solvents were added, the mixtures (or solutions) were stirred at room temperature. When slurries were formed, the solids were filtered for XRPD analysis.

XRPD analysis was done in three stages. XRPD of the wet cake was done for all collected samples. Unique solids were then left on XRPD plates and dried under vacuum at 50° C. XRPD of unique dry solids was then done. Solids were then exposed to 95%+ relative humidity for one day and XRPD on resulting solids was done. The humid environment was generated by placing a beaker of saturated potassium sulfate in water in a sealed container. All XRPD patterns were compared to counter ion XRPD patterns (if solid). A summary of the results is given in Table 26. The naming scheme of the crystalline salt forms is the counter ion number followed by a letter corresponding to the unique pattern observed for that counter ion. For example, Form 8-B would designate the second unique pattern observed with the L-malic acid counter ion.

TABLE 26

| | Slurry | XRPD Form | | |
|---|---|---|---|---|
| Counter Ion | Solvent | Wet | dry | humidity |
| Benzenesulfonic | EtOAc | Am | Am | Am |
| acid (1) | EtOH | 1-A | 1-A | Am |
| | IPA:water | 1-B | 1-B | 1-B |
| | (9:1) | | | |
| Citric acid | EtOAc | 2-A | 2-A | 2-C |
| (2) | EtOH | 2-B | 2-B | 2-C |
| | IPA:water | 2-C | 2-B | 2-C |
| | (9:1) | | | |
| Fumaric acid | EtOAc | 3-A | 3-A | 3-A |
| (3) | EtOH | 3-A | 3-A | — |
| | IPA:water | 3-A | 3-A | — |
| | (9:1) | | | |
| Hydrochloric | EtOAc | 5-C | 5-C | 5C + 5A |
| acid (5) | EtOH | Am | Am | Am |
| | IPA:water | 5-B | 5-B | 5-B |
| | (9:1) | | | |
| Maleic acid | EtOAc | 7-A | 7-A | 7-A |
| (7) | EtOH | 7-A + P | 7-A + P | 7-A + P |
| | IPA:water | 7-A | 7-A | 7-A |
| | (9:1) | | | |
| L-Malic acid | EtOAc | 8-A | 8-A | 8-A |
| (8) | EtOH | 8-A | 8-A | 8-A |
| | IPA:water | 8-B | 8-B | 8-B |
| | (9:1) | | | |
| Methanesulfonic | EtOAc | 9-A | 9-C | 9C + 9B |
| acid (9) | EtOH | 9-B | 9-1 | 9-B |
| | IPA:water | 9-B | 9-B | 9-B |
| | (9:1) | | | |
| Phosphoric acid | EtOAc | 10-A | 10-A | 10-A |
| (10) | EtOH | 10-A | 10-A | — |
| | IPA:water | 10-A | 10-A | — |
| | (9:1) | | | |
| Pyruvic acid | EtOAc | 11-A | 11-A | 11-A |
| (11) | EtOH | 11-A | 11-A | — |
| | IPA:water | Am | Am | — |
| | (9:1) | | | |
| Sulfuric acid | EtOAc | 12-A | 12-A | 12-A + 12-B |
| (12) | EtOH | Am | Am | Am |
| | IPA:water | 12-A + 12-B | 12-A + 12-B | 12-A + 12-B |
| | (9:1) | | | |
| L-Tartaric acid | EtOAc | 13-A | 13-A | 13-A |
| (13) | EtOH | 13-A | 13-A | — |
| | IPA:water | 13-B | 13-B | 13-B |
| | (9:1) | | | |
| Toluenesulfonic | EtOAc | 14-A | 14-D | 14-B |
| acid (14) | EtOH | 14-B | 14-B | — |
| | IPA:water | 14-B | 14-B | 14-B |
| | (9:1) | | | |

Am = amorphous

The XRPD pattern of crystalline salt Form 1-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, is shown in FIG. 30. Characteristic peaks include one or more of the peaks shown in Table 27.

TABLE 27

| | | Relative |
|---|---|---|
| | d-spacing | intensity |
| 2θ (deg) | (Å) | (a.u) |
| 5.14 | 17.20 | 53 |
| 6.49 | 13.60 | 100 |
| 6.76 | 13.06 | 62 |
| 7.05 | 12.53 | 60 |
| 8.16 | 10.83 | 6 |
| 9.27 | 9.53 | 6 |
| 9.62 | 9.19 | 16 |
| 10.16 | 8.70 | 7 |
| 12.09 | 7.32 | 16 |
| 13.44 | 6.58 | 7 |
| 14.04 | 6.30 | 9 |
| 14.83 | 5.97 | 15 |
| 15.08 | 5.87 | 38 |
| 15.50 | 5.71 | 34 |
| 16.39 | 5.40 | 14 |
| 17.02 | 5.21 | 6 |
| 18.57 | 4.77 | 18 |
| 19.33 | 4.59 | 10 |
| 20.16 | 4.40 | 11 |
| 21.30 | 4.17 | 19 |
| 24.22 | 3.67 | 10 |
| 24.45 | 3.64 | 7 |
| 24.87 | 3.58 | 7 |
| 25.85 | 3.44 | 5 |
| 26.12 | 3.41 | 5 |
| 28.14 | 3.17 | 5 |

The XRPD pattern of crystalline salt Form 1-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, benzenesulfonic acid salt, is shown in FIG. 31. Characteristic peaks include one or more of the peaks shown in Table 28.

TABLE 28

| | | Relative |
|---|---|---|
| | d-spacing | intensity |
| 2θ (deg) | (Å) | (a.u) |
| 4.13 | 21.37 | 7 |
| 5.35 | 16.50 | 12 |
| 6.17 | 14.31 | 31 |
| 6.71 | 13.17 | 100 |
| 8.19 | 10.79 | 18 |
| 8.53 | 10.36 | 13 |
| 10.04 | 8.80 | 31 |
| 10.63 | 8.31 | 36 |
| 12.30 | 7.19 | 12 |
| 12.58 | 7.03 | 9 |
| 13.42 | 6.60 | 5 |
| 14.03 | 6.31 | 26 |
| 14.61 | 6.06 | 7 |
| 15.61 | 5.67 | 19 |
| 15.97 | 5.55 | 17 |
| 16.21 | 5.46 | 27 |
| 17.29 | 5.13 | 5 |
| 18.48 | 4.80 | 12 |
| 21.29 | 4.17 | 5 |
| 21.75 | 4.08 | 9 |
| 22.15 | 4.01 | 14 |
| 22.74 | 3.91 | 11 |
| 23.15 | 3.84 | 5 |
| 24.14 | 3.68 | 11 |
| 25.94 | 3.43 | 7 |

FIG. 32 depicts the differential scanning calorimetry (DSC) profile of crystalline salt Form 1-B. As shown in FIG. 32, crystalline salt Form 1-B shows a characteristic endotherm with an onset of about 33° C. and a peak of about 71° C., a characteristic endotherm with an onset of about 120° C. and a peak of about 133° C., and a characteristic endotherm with an onset of about 154° C. and a peak of about 159° C.

The XRPD pattern of crystalline salt Form 2-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is shown in FIG. 33. Characteristic peaks include one or more of the peaks shown in Table 29.

TABLE 29

| | | Relative |
|---|---|---|
| | d-spacing | intensity |
| 2θ (deg) | (Å) | (a.u) |
| 4.73 | 18.65 | 22 |
| 5.91 | 14.93 | 5 |
| 7.03 | 12.56 | 100 |
| 7.89 | 11.20 | 62 |
| 9.14 | 9.67 | 10 |
| 10.25 | 8.63 | 14 |
| 11.40 | 7.76 | 45 |
| 12.55 | 7.05 | 19 |
| 13.71 | 6.46 | 11 |
| 14.14 | 6.26 | 9 |
| 15.10 | 5.86 | 21 |
| 16.93 | 5.23 | 5 |
| 18.34 | 4.83 | 6 |
| 19.05 | 4.66 | 9 |
| 20.37 | 4.36 | 6 |
| 20.94 | 4.24 | 5 |

The XRPD pattern of crystalline salt Form 2-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is shown in FIG. 34. Characteristic peaks include one or more of the peaks shown in Table 30.

TABLE 30

| | | Relative |
|---|---|---|
| | d-spacing | intensity |
| 2θ (deg) | (Å) | (a.u) |
| 4.69 | 18.84 | 6 |
| 6.76 | 13.07 | 23 |
| 7.88 | 11.21 | 31 |
| 9.29 | 9.51 | 89 |
| 11.17 | 7.92 | 14 |
| 12.53 | 7.06 | 49 |
| 13.85 | 6.39 | 21 |
| 15.19 | 5.83 | 100 |
| 17.00 | 5.21 | 48 |
| 18.45 | 4.81 | 35 |
| 19.47 | 4.55 | 51 |
| 20.36 | 4.36 | 5 |
| 22.27 | 3.99 | 13 |
| 24.24 | 3.67 | 8 |
| 26.15 | 3.41 | 11 |
| 28.47 | 3.13 | 14 |

The XRPD pattern of crystalline salt Form 2-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, citric acid salt, is shown in FIG. 35. Characteristic peaks include one or more of the peaks shown in Table 31.

TABLE 31

| | | Relative |
|---|---|---|
| | d-spacing | intensity |
| 2θ (deg) | (Å) | (a.u) |
| 6.72 | 13.14 | 23 |
| 7.78 | 11.35 | 34 |
| 9.12 | 9.69 | 78 |
| 11.06 | 7.99 | 6 |
| 12.55 | 7.05 | 47 |
| 13.63 | 6.49 | 12 |
| 14.27 | 6.20 | 8 |
| 15.00 | 5.90 | 100 |
| 16.92 | 5.24 | 38 |
| 18.17 | 4.88 | 20 |
| 18.49 | 4.80 | 6 |
| 19.22 | 4.61 | 48 |
| 20.25 | 4.38 | 5 |
| 22.08 | 4.02 | 15 |
| 24.00 | 3.71 | 5 |
| 28.02 | 3.18 | 8 |
| 28.65 | 3.11 | 8 |

FIG. 36 depicts the differential scanning calorimetry (DSC) profile of crystalline salt Form 2-C. As shown in FIG. 36, crystalline salt Form 2-C shows a characteristic endotherm with an onset of about 33° C. and a peak of about 60° C., a characteristic endotherm with an onset of about 96° C. and a peak of about 116° C., a characteristic endotherm with an onset of about 160° C. and a peak of about 169° C., and a characteristic endotherm with an onset of about 141° C. and a peak of about 177° C.

The XRPD pattern of crystalline salt Form 3-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, fumaric acid salt, is shown in FIG. 37. Characteristic peaks include one or more of the peaks shown in Table 32.

TABLE 32

| | | Relative |
|---|---|---|
| | d-spacing | intensity |
| 2θ (deg) | (Å) | (a.u) |
| 7.11 | 12.42 | 100 |
| 8.24 | 10.72 | 99 |
| 10.91 | 8.11 | 10 |
| 14.30 | 6.19 | 67 |
| 16.49 | 5.37 | 47 |
| 18.04 | 4.91 | 15 |
| 18.92 | 4.69 | 25 |
| 20.91 | 4.25 | 7 |
| 21.43 | 4.14 | 7 |
| 21.88 | 4.06 | 33 |
| 22.14 | 4.01 | 30 |
| 22.42 | 3.96 | 13 |
| 22.99 | 3.86 | 9 |
| 23.58 | 3.77 | 6 |
| 24.02 | 3.70 | 10 |
| 24.46 | 3.64 | 28 |
| 24.82 | 3.58 | 29 |
| 25.36 | 3.51 | 22 |
| 26.45 | 3.37 | 5 |
| 26.96 | 3.30 | 9 |
| 27.19 | 3.28 | 10 |
| 27.50 | 3.24 | 6 |
| 28.55 | 3.12 | 5 |

FIG. 38 depicts the differential scanning calorimetry (DSC) profile of crystalline salt Form 3-A. As shown in FIG. 38, crystalline salt Form 3-A shows a characteristic endotherm with an onset of about 237° C. and a peak of about 241° C.

The XRPD pattern of crystalline salt Form 5-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is shown in FIG. 39. Characteristic peaks include one or more of the peaks shown in Table 33.

TABLE 33

| | | Relative |
|---|---|---|
| | d-spacing | intensity |
| 2θ (deg) | (Å) | (a.u) |
| 5.04 | 17.53 | 9 |
| 6.00 | 14.72 | 8 |
| 7.82 | 11.30 | 100 |
| 8.10 | 10.90 | 10 |
| 9.32 | 9.48 | 49 |
| 11.71 | 7.55 | 9 |
| 12.23 | 7.23 | 15 |
| 12.91 | 6.85 | 9 |
| 13.46 | 6.57 | 12 |
| 13.90 | 6.36 | 12 |
| 14.14 | 6.26 | 15 |
| 15.16 | 5.84 | 5 |
| 15.79 | 5.61 | 8 |
| 16.21 | 5.46 | 7 |
| 17.84 | 4.97 | 5 |
| 19.26 | 4.60 | 10 |
| 20.12 | 4.41 | 7 |
| 20.33 | 4.36 | 10 |
| 21.30 | 4.17 | 9 |
| 22.10 | 4.02 | 5 |
| 22.33 | 3.98 | 6 |
| 22.82 | 3.89 | 6 |
| 23.27 | 3.82 | 8 |
| 23.50 | 3.78 | 9 |
| 24.02 | 3.70 | 5 |
| 25.19 | 3.53 | 10 |
| 25.68 | 3.47 | 6 |
| 25.94 | 3.43 | 6 |
| 26.45 | 3.37 | 5 |
| 27.19 | 3.28 | 7 |

The contemplated crystalline Form 5-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloric acid salt, displayed a thermogravimetric analysis (TGA) profile showing a mass loss of about 3.1 wt. % up to about 110° C. and a further mass loss of about 7.6 wt. % between about 110° C. to about 195° C. The crystalline Form 5-A displayed a dynamic vapor sorption (DVS) profile showing a reversable total mass change of about 3.1 wt. % between about 2 to about 92% relative humidity (RH) at 25° C. Crystalline Form 5-A may be characterized by a Karl-Fischer titration profile showing a water content of about 10.7%. Crystalline Form 5-A displayed a rod-like or plate-like morphology by optical microscopy.

The XRPD pattern of crystalline salt Form 5-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is shown in FIG. 40. Characteristic peaks include one or more of the peaks shown in Table 34.

TABLE 34

| | | Relative |
|---|---|---|
| | d-spacing | intensity |
| 2θ (deg) | (Å) | (a.u) |
| 7.42 | 11.90 | 22 |
| 9.15 | 9.66 | 100 |
| 9.88 | 8.95 | 23 |
| 11.71 | 7.55 | 22 |
| 14.18 | 6.24 | 20 |
| 14.63 | 6.05 | 5 |
| 17.48 | 5.07 | 10 |
| 20.01 | 4.43 | 7 |
| 20.67 | 4.29 | 7 |
| 21.22 | 4.18 | 12 |
| 21.83 | 4.07 | 11 |
| 23.07 | 3.85 | 7 |
| 23.93 | 3.72 | 10 |
| 24.27 | 3.66 | 5 |
| 25.32 | 3.51 | 7 |
| 25.73 | 3.46 | 11 |
| 27.56 | 3.23 | 35 |
| 29.39 | 3.04 | 6 |

FIG. 41 depicts the differential scanning calorimetry (DSC) profile of crystalline salt Form 5-B. As shown in FIG. 41, crystalline salt Form 5-B shows a characteristic endotherm with an onset of about 68° C. and a peak of about 82° C., a characteristic endotherm with an onset of about 111° C. and a peak of about 130° C., and a characteristic endotherm with an onset of about 193° C. and a peak of about 211° C.

The XRPD pattern of crystalline salt Form 5-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is shown in FIG. 42. Characteristic peaks include one or more of the peaks shown in Table 35.

TABLE 35

| | | Relative |
|---|---|---|
| | d-spacing | intensity |
| 2θ (deg) | (Å) | (a.u) |
| 10.71 | 8.25 | 8 |
| 11.78 | 7.51 | 13 |
| 12.90 | 6.86 | 5 |
| 13.11 | 6.75 | 5 |
| 16.18 | 5.47 | 12 |
| 18.12 | 4.89 | 13 |
| 20.50 | 4.33 | 11 |
| 21.52 | 4.13 | 100 |
| 22.43 | 3.96 | 23 |
| 23.92 | 3.72 | 38 |
| 26.41 | 3.37 | 8 |
| 26.62 | 3.35 | 5 |
| 26.97 | 3.30 | 15 |
| 28.93 | 3.08 | 15 |

FIG. 43 depicts the differential scanning calorimetry (DSC) profile of crystalline salt Form 5-C. As shown in FIG. 43, crystalline salt Form 5-C shows a characteristic endotherm with an onset of about 140° C. and a peak of about 145° C., and a characteristic endotherm with an onset of about 213° C. and a peak of about 230° C.

Crystalline, Form 5-D material of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, was prepared as follows. Form 5-B material was slurried in IPA:water (9:1 vol.) by adding 50 volumes. The slurry was sonicated in a sonicating bath for 5.5 hours, keeping the bath temperature between 17° C. and 31° C. The solids were collected by filtration and analyzed by XRPD. XRPD analysis indicated that the material was crystalline with a pattern consistent with Form 5-D.

The XRPD pattern of crystalline salt Form 5-D of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, hydrochloride salt, is shown in FIG. 44. Characteristic peaks include one or more of the peaks shown in Table 36.

TABLE 36

| | | Relative |
|---|---|---|
| | d-spacing | intensity |
| 2θ (deg) | (Å) | (a.u) |
| 7.79 | 11.34 | 5 |
| 8.44 | 10.47 | 100 |
| 12.72 | 6.95 | 19 |
| 14.01 | 6.32 | 18 |
| 14.99 | 5.91 | 11 |
| 16.99 | 5.21 | 7 |
| 22.93 | 3.88 | 11 |
| 25.62 | 3.47 | 8 |
| 26.03 | 3.42 | 9 |

The XRPD pattern of crystalline salt Form 7-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, maleic acid salt, is shown in FIG. 45. Characteristic peaks include one or more of the peaks shown in Table 37.

TABLE 37

| | | Relative |
|---|---|---|
| | d-spacing | intensity |
| 2θ (deg) | (Å) | (a.u) |
| 7.25 | 12.18 | 55 |
| 8.34 | 10.60 | 58 |
| 10.96 | 8.07 | 12 |
| 14.33 | 6.17 | 19 |
| 14.91 | 5.94 | 87 |
| 16.53 | 5.36 | 28 |
| 18.94 | 4.68 | 25 |
| 21.47 | 4.14 | 100 |
| 21.87 | 4.06 | 9 |
| 22.39 | 3.97 | 16 |
| 22.73 | 3.91 | 29 |
| 23.05 | 3.86 | 12 |
| 23.48 | 3.79 | 12 |
| 24.18 | 3.68 | 11 |
| 24.57 | 3.62 | 11 |
| 24.93 | 3.57 | 15 |
| 25.27 | 3.52 | 29 |
| 25.89 | 3.44 | 19 |
| 27.16 | 3.28 | 22 |
| 27.50 | 3.24 | 19 |
| 27.90 | 3.19 | 10 |
| 29.02 | 3.07 | 6 |
| 29.43 | 3.03 | 6 |

FIG. 46 depicts the differential scanning calorimetry (DSC) profile of crystalline salt Form 7-A. As shown in FIG. 46, crystalline salt Form 7-A shows a characteristic endotherm with an onset of about 215° C. and a peak of about 221° C., and a characteristic endotherm with an onset of about 216° C. and a peak of about 225° C.

The XRPD pattern of crystalline salt Form 8-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, is shown in FIG. 47. Characteristic peaks include one or more of the peaks shown in Table 38.

TABLE 38

| | | Relative |
|---|---|---|
| | d-spacing | intensity |
| 2θ (deg) | (Å) | a.u |
| 7.27 | 12.14 | 78 |
| 8.30 | 10.64 | 18 |
| 10.98 | 8.05 | 16 |
| 14.33 | 6.17 | 26 |
| 15.00 | 5.90 | 100 |
| 16.60 | 5.34 | 51 |
| 19.01 | 4.66 | 33 |
| 20.53 | 4.32 | 5 |
| 20.93 | 4.24 | 5 |
| 21.64 | 4.10 | 82 |
| 22.03 | 4.03 | 13 |
| 22.72 | 3.91 | 6 |
| 23.87 | 3.72 | 5 |
| 24.02 | 3.70 | 6 |
| 24.68 | 3.60 | 21 |
| 25.48 | 3.49 | 8 |
| 27.29 | 3.27 | 39 |
| 28.92 | 3.09 | 5 |
| 29.23 | 3.05 | 5 |

FIG. 48 depicts the differential scanning calorimetry (DSC) profile of crystalline salt Form 8-A. As shown in FIG. 48, crystalline salt Form 8-A shows a characteristic endotherm with an onset of about 177° C. and a peak of about 201° C., a characteristic endotherm with an onset of about 186° C. and a peak of about 207° C., a characteristic endotherm with an onset of about 205° C. and a peak of about 211° C., and a characteristic endotherm with an onset of about 208° C. and a peak of about 216° C.

The XRPD pattern of crystalline salt Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, is shown in FIG. 49. Characteristic peaks include one or more of the peaks shown in Table 39.

TABLE 39

| | | Relative |
|---|---|---|
| | d-spacing | intensity |
| 2θ (deg) | (Å) | a.u |
| 5.88 | 15.03 | 100 |
| 9.13 | 9.68 | 50 |
| 11.60 | 7.62 | 10 |
| 12.97 | 6.82 | 8 |
| 16.33 | 5.43 | 91 |
| 17.62 | 5.03 | 17 |
| 18.18 | 4.88 | 14 |
| 19.14 | 4.63 | 30 |
| 21.17 | 4.19 | 13 |
| 22.04 | 4.03 | 5 |
| 22.86 | 3.89 | 59 |
| 23.78 | 3.74 | 24 |
| 25.73 | 3.46 | 5 |
| 27.10 | 3.29 | 13 |
| 28.37 | 3.14 | 38 |
| 29.10 | 3.07 | 12 |

FIG. 50 depicts the differential scanning calorimetry (DSC) profile of crystalline salt Form 8-B. As shown in FIG. 50, crystalline salt Form 8-B shows a characteristic endotherm with an onset of about 189° C. and a peak of about 192° C., and a characteristic endotherm with an onset of about 186° C. and a peak of about 202° C.

Crystalline Form 8-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-malic acid salt, displayed a thermogravimetric analysis (TGA) profile showing a mass loss of about 0.46 wt. % up to about 175° C. Crystalline Form 8-B may be characterized by a dynamic vapor sorption (DVS) profile showing a reversable total mass change of about 0.82 wt. % between about 2 to about 92% relative humidity (RH) at 25° C.

The XRPD pattern of crystalline salt Form 9-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is shown in FIG. 51. Characteristic peaks include one or more of the peaks shown in Table 40.

TABLE 40

| | | Relative |
|---|---|---|
| | d-spacing | intensity |
| 2θ (deg) | (Å) | a.u |
| 4.55 | 19.39 | 26 |
| 5.45 | 16.21 | 22 |
| 6.09 | 14.51 | 10 |
| 7.53 | 11.73 | 78 |
| 8.18 | 10.81 | 7 |
| 9.07 | 9.74 | 32 |
| 9.47 | 9.33 | 100 |
| 10.10 | 8.75 | 22 |
| 10.81 | 8.18 | 15 |
| 12.81 | 6.90 | 16 |
| 13.41 | 6.60 | 18 |
| 14.73 | 6.01 | 38 |
| 14.97 | 5.91 | 50 |
| 15.29 | 5.79 | 21 |
| 15.72 | 5.63 | 25 |
| 16.18 | 5.47 | 7 |
| 17.59 | 5.04 | 2 |
| 18.03 | 4.92 | 41 |
| 18.60 | 4.77 | 9 |
| 18.77 | 4.72 | 5 |
| 20.12 | 4.41 | 28 |
| 21.55 | 4.12 | 37 |
| 22.36 | 3.97 | 15 |
| 22.95 | 3.87 | 9 |
| 24.18 | 3.68 | 8 |
| 24.88 | 3.58 | 7 |
| 25.47 | 3.49 | 9 |
| 25.75 | 3.46 | 6 |
| 27.10 | 3.29 | 11 |
| 27.57 | 3.23 | 11 |

The XRPD pattern of crystalline salt Form 9-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is shown in FIG. 52. Characteristic peaks include one or more of the peaks shown in Table 41.

TABLE 41

| | | Relative |
|---|---|---|
| | d-spacing | intensity |
| 2θ (deg) | (Å) | a.u |
| 8.16 | 10.83 | 100 |
| 8.71 | 10.14 | 10 |
| 13.71 | 6.46 | 21 |
| 14.71 | 6.02 | 24 |
| 16.21 | 5.46 | 24 |
| 18.89 | 4.69 | 43 |
| 22.31 | 3.98 | 14 |
| 22.77 | 3.90 | 9 |
| 24.37 | 3.65 | 6 |
| 25.72 | 3.46 | 5 |
| 26.98 | 3.30 | 11 |
| 27.49 | 3.24 | 6 |
| 28.86 | 3.09 | 6 |

FIG. 53 depicts the differential scanning calorimetry (DSC) profile of crystalline salt Form 9-B. As shown in FIG. 53, crystalline salt Form 9-B shows a characteristic endotherm with an onset of about 189° C. and a peak of about 192° C., and a characteristic endotherm with an onset of about 186° C. and a peak of about 202° C.

The XRPD pattern of crystalline salt Form 9-C of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, methanesulfonic acid salt, is shown in FIG. 54. Characteristic peaks include one or more of the peaks shown in Table 42.

TABLE 42

| | | Relative |
|---|---|---|
| | d-spacing | intensity |
| 2θ (deg) | (Å) | a.u |
| 4.55 | 19.41 | 28 |
| 5.47 | 16.13 | 13 |
| 6.14 | 14.37 | 8 |
| 7.56 | 11.68 | 75 |
| 8.17 | 10.82 | 8 |
| 9.19 | 9.61 | 33 |
| 9.50 | 9.30 | 100 |
| 10.23 | 8.64 | 21 |
| 10.89 | 8.12 | 21 |
| 12.74 | 6.94 | 13 |
| 13.01 | 6.80 | 15 |
| 13.44 | 6.58 | 18 |
| 14.77 | 5.99 | 30 |
| 15.03 | 5.89 | 41 |
| 15.39 | 5.75 | 18 |
| 15.93 | 5.56 | 25 |
| 16.32 | 5.43 | 8 |
| 18.22 | 4.86 | 33 |
| 18.80 | 4.72 | 12 |
| 20.06 | 4.42 | 16 |
| 20.42 | 4.34 | 20 |
| 21.79 | 4.08 | 29 |
| 22.40 | 3.97 | 12 |
| 23.23 | 3.83 | 7 |
| 24.34 | 3.65 | 8 |
| 25.02 | 3.56 | 7 |
| 25.41 | 3.50 | 7 |
| 25.71 | 3.46 | 6 |
| 26.15 | 3.40 | 6 |
| 27.40 | 3.25 | 8 |
| 27.55 | 3.23 | 9 |

The XRPD pattern of crystalline salt Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, is shown in FIG. 55. Characteristic peaks include one or more of the peaks shown in Table 43.

TABLE 43

| | | Relative |
|---|---|---|
| | d-spacing | intensity |
| 2θ (deg) | (Å) | a.u |
| 6.17 | 14.31 | 28 |
| 8.55 | 10.34 | 21 |
| 10.21 | 8.66 | 10 |
| 10.76 | 8.21 | 20 |
| 11.46 | 7.71 | 23 |
| 12.29 | 7.20 | 100 |
| 15.67 | 5.65 | 14 |
| 16.76 | 5.29 | 10 |
| 17.07 | 5.19 | 5 |
| 18.42 | 4.81 | 13 |
| 20.55 | 4.32 | 30 |
| 21.19 | 4.19 | 12 |
| 22.88 | 3.88 | 30 |
| 24.23 | 3.67 | 5 |
| 24.63 | 3.61 | 8 |
| 25.77 | 3.45 | 30 |
| 26.72 | 3.33 | 5 |

FIG. 56 depicts the differential scanning calorimetry (DSC) profile of crystalline salt Form 10-A. As shown in FIG. 56, crystalline salt Form 10-A shows a characteristic endotherm with an onset of about 282° C. and a peak of about 290° C., and a characteristic endotherm with an onset of about 283° C. and a peak of about 294° C.

Crystalline Form 10-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, phosphoric acid salt, displayed a thermogravimetric analysis (TGA) profile showing a mass loss of about 0.22 wt. %. Crystalline Form 10-A displayed a dynamic vapor sorption (DVS) profile showing a reversable total mass change of about 1.4 wt. % between about 2 to about 92% relative humidity (RH) at 25° C.

The XRPD pattern of crystalline salt Form 11-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, pyruvic acid salt, is shown in FIG. 57. Characteristic peaks include one or more of the peaks shown in Table 44.

TABLE 44

| | | Relative |
|---|---|---|
| | d-spacing | intensity |
| 2θ (deg) | (Å) | a.u |
| 4.38 | 20.18 | 46 |
| 4.92 | 17.93 | 24 |
| 7.22 | 12.24 | 44 |
| 7.53 | 11.73 | 100 |
| 8.64 | 10.23 | 51 |
| 9.10 | 9.71 | 32 |
| 9.90 | 8.93 | 11 |
| 11.43 | 7.73 | 20 |
| 13.06 | 6.77 | 12 |
| 14.51 | 6.10 | 15 |
| 14.67 | 6.03 | 19 |
| 17.16 | 5.16 | 7 |
| 20.06 | 4.42 | 5 |
| 20.49 | 4.33 | 6 |

FIG. 58 depicts the differential scanning calorimetry (DSC) profile of crystalline salt Form 11-A. As shown in FIG. 58, crystalline salt Form 11-A shows a characteristic endotherm with an onset of about 76° C. and a peak of about 88° C., a characteristic endotherm with an onset of about 134° C. and a peak of about 142° C., and a characteristic endotherm with an onset of about 149° C. and a peak of about 157° C.

The XRPD pattern of crystalline salt Form 12-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, sulfuric acid salt, is shown in FIG. 59. Characteristic peaks include one or more of the peaks shown in Table 45.

TABLE 45

| | | Relative |
|---|---|---|
| | d-spacing | intensity |
| 2θ (deg) | (Å) | a.u |
| 4.38 | 20.16 | 6 |
| 4.58 | 19.27 | 8 |
| 5.11 | 17.30 | 14 |
| 5.95 | 14.83 | 100 |
| 6.65 | 13.29 | 46 |
| 7.04 | 12.55 | 23 |
| 7.95 | 11.12 | 67 |
| 8.16 | 10.83 | 53 |
| 8.63 | 10.24 | 12 |
| 8.86 | 9.97 | 17 |
| 9.58 | 9.23 | 56 |
| 9.75 | 9.06 | 10 |
| 10.45 | 8.46 | 6 |
| 11.07 | 7.99 | 16 |
| 11.81 | 7.49 | 97 |
| 12.31 | 7.18 | 11 |
| 12.87 | 6.87 | 6 |
| 13.30 | 6.65 | 9 |
| 13.49 | 6.56 | 30 |
| 13.81 | 6.41 | 35 |
| 14.18 | 6.24 | 10 |
| 14.43 | 6.14 | 30 |
| 14.75 | 6.00 | 62 |
| 15.27 | 5.80 | 58 |
| 16.26 | 5.45 | 32 |
| 17.13 | 5.17 | 54 |
| 17.66 | 5.02 | 39 |
| 18.12 | 4.89 | 21 |
| 18.89 | 4.70 | 48 |
| 19.79 | 4.48 | 9 |
| 20.16 | 4.40 | 12 |
| 20.68 | 4.29 | 32 |
| 21.14 | 4.20 | 31 |
| 21.47 | 4.14 | 20 |
| 22.60 | 3.93 | 68 |
| 22.60 | 3.93 | 68 |
| 23.45 | 3.79 | 20 |
| 23.66 | 3.76 | 11 |
| 24.09 | 3.69 | 20 |
| 24.48 | 3.63 | 12 |
| 24.93 | 3.57 | 32 |
| 25.53 | 3.49 | 23 |
| 26.37 | 3.38 | 15 |
| 26.42 | 3.37 | 14 |
| 27.00 | 3.30 | 20 |
| 27.74 | 3.21 | 18 |
| 29.08 | 3.07 | 15 |
| 29.38 | 3.04 | 8 |

FIG. 60 depicts the differential scanning calorimetry (DSC) profile of crystalline salt Form 12-A. As shown in FIG. 58, crystalline salt Form 12-A shows a characteristic endotherm with an onset of about 48° C. and a peak of about 81° C., a characteristic endotherm with an onset of about 169° C. and a peak of about 185° C., and a characteristic endotherm with an onset of about 229° C. and a peak of about 241° C.

The XRPD pattern of crystalline salt Form 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, is shown in FIG. 61. Characteristic peaks include one or more of the peaks shown in Table 46.

TABLE 46

| | | Relative |
|---|---|---|
| | d-spacing | intensity |
| 2θ (deg) | (Å) | a.u |
| 7.41 | 11.91 | 6 |
| 7.82 | 11.30 | 29 |
| 8.02 | 11.01 | 38 |
| 8.72 | 10.14 | 45 |
| 10.69 | 8.27 | 5 |
| 11.10 | 7.96 | 54 |
| 14.23 | 6.22 | 59 |
| 15.43 | 5.74 | 24 |
| 15.91 | 5.57 | 100 |
| 17.33 | 5.11 | 69 |
| 19.50 | 4.55 | 94 |
| 20.22 | 4.39 | 8 |
| 20.65 | 4.30 | 27 |
| 21.56 | 4.12 | 40 |
| 23.14 | 3.84 | 69 |
| 23.56 | 3.77 | 30 |
| 24.42 | 3.64 | 28 |
| 24.86 | 3.58 | 9 |
| 25.25 | 3.52 | 5 |
| 26.14 | 3.41 | 23 |
| 26.92 | 3.31 | 41 |
| 28.19 | 3.16 | 40 |
| 29.15 | 3.06 | 5 |
| 29.66 | 3.01 | 5 |

FIG. 62 depicts the differential scanning calorimetry (DSC) profile of crystalline salt Form 13-A. As shown in FIG. 62, crystalline salt Form 13-A shows a characteristic endotherm with an onset of about 213° C. and a peak of about 222° C.

Crystalline 13-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, displayed a thermogravimetric analysis (TGA) profile showing a mass loss of about 0.63 wt. % up to about 185° C. Crystalline Form 13-A displayed a dynamic vapor sorption (DVS) profile showing a reversable total mass change of about 0.97 wt. % between about 2 to about 92% relative humidity (RH) at 25° C.

The XRPD pattern of crystalline salt Form 13-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, L-tartaric acid salt, is shown in FIG. 63. Characteristic peaks include one or more of the peaks shown in Table 47.

TABLE 47

| | | Relative |
|---|---|---|
| | d-spacing | intensity |
| 2θ (deg) | (Å) | a.u |
| 7.08 | 12.47 | 41 |
| 8.67 | 10.20 | 8 |
| 11.79 | 7.50 | 6 |
| 16.88 | 5.25 | 100 |
| 20.85 | 4.26 | 8 |
| 21.85 | 4.06 | 32 |
| 22.18 | 4.00 | 13 |
| 24.06 | 3.70 | 10 |
| 25.14 | 3.54 | 5 |
| 25.31 | 3.52 | 5 |
| 27.01 | 3.30 | 14 |
| 27.70 | 3.22 | 16 |

FIG. 64 depicts the differential scanning calorimetry (DSC) profile of crystalline salt Form 13-B. As shown in FIG. 64, crystalline salt Form 13-B shows a characteristic endotherm with an onset of about 89° C. and a peak of about 115° C., a characteristic endotherm with an onset of about 157° C. and a peak of about 167° C., and a characteristic endotherm with an onset of about 181° C. and a peak of about 195° C.

The XRPD pattern of crystalline salt Form 14-A of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, is shown in FIG. 65. Characteristic peaks include one or more of the peaks shown in Table 48.

TABLE 48

| | | Relative |
|---|---|---|
| | d-spacing | intensity |
| 2θ (deg) | (Å) | a.u |
| 4.45 | 19.85 | 63 |
| 4.73 | 18.68 | 7 |
| 6.12 | 14.44 | 68 |
| 6.36 | 13.89 | 11 |
| 6.74 | 13.11 | 23 |
| 8.02 | 11.01 | 6 |
| 8.76 | 10.09 | 54 |
| 9.50 | 9.30 | 66 |
| 10.32 | 8.56 | 35 |
| 12.17 | 7.27 | 4 |
| 13.06 | 6.77 | 5 |
| 13.10 | 6.75 | 7 |
| 14.28 | 6.20 | 7 |
| 15.31 | 5.78 | 34 |
| 16.87 | 5.25 | 5 |
| 17.48 | 5.07 | 100 |
| 18.56 | 4.78 | 8 |
| 18.97 | 4.68 | 8 |
| 19.81 | 4.48 | 6 |
| 20.66 | 4.30 | 8 |
| 21.30 | 4.17 | 26 |
| 21.89 | 4.06 | 54 |
| 27.19 | 3.28 | 29 |

The XRPD pattern of crystalline salt Form 14-B of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, toluenesulfonic acid salt, is shown in FIG. 66. Characteristic peaks include one or more of the peaks shown in Table 49.

TABLE 49

| | | Relative |
|---|---|---|
| | d-spacing | intensity |
| 2θ (deg) | (Å) | a.u |
| 6.78 | 13.02 | 57 |
| 7.67 | 11.52 | 100 |
| 12.89 | 6.86 | 33 |
| 15.30 | 5.79 | 22 |
| 17.24 | 5.14 | 26 |
| 21.41 | 4.15 | 56 |
| 22.41 | 3.96 | 55 |
| 24.70 | 3.60 | 80 |
| 25.91 | 3.44 | 41 |

### Example 25. Crystallization Study

The objective of this study was to develop a reliable crystallization process with repeatable particle size distribution (PSD) control. Solubility measurements of Form P were performed in a DMSO/EtOH/Water system at evaluated temperatures. Form P was slurried in the selected solvent systems for 2-3 hours, then the mother liquors were collected for solubility test by a high-performance liquid chromatography (HPLC) assay. The results in Table 50 show that the solubility decreased significantly with water content increasing.

TABLE 50

| | Temp | Solubility |
|---|---|---|
| Solvent (v/v) | (° C.) | (mg/mL) |
| EtOH/DMSO = 8/8 | 25 ± 5 | 157.2 |
| (EtOH/DMSO = 1/1)/H₂O = 10/1 | 25 ± 5 | 81.6 |
| (EtOH/DMSO = 1/1)/H₂O = 10/2 | 25 ± 5 | 53.8 |
| (EtOH/DMSO = 1/1)/H₂O = 10/3 | 25 ± 5 | 37.9 |
| (EtOH/DMSO = 1/1)/H₂O = 10/5 | 25 ± 5 | 19.7 |
| (EtOH/DMSO = 1/1)/H₂O = 10/10 | 25 ± 5 | 5.3 |
| (EtOH/DMSO = 1/1)/H₂O = 10/20 | 25 ± 5 | 0.8 |
| (EtOH/DMSO = 1/1)/H₂O = 10/40 | 25 ± 5 | 0.1 |
| EtOH/DMSO = 8/8 | 50 ± 5 | >200 |
| (EtOH/DMSO = 1/1)/H₂O = 10/1 | 50 ± 5 | 123.6 |
| (EtOH/DMSO = 1/1)/H₂O = 10/2 | 50 ± 5 | 87.0 |
| (EtOH/DMSO = 1/1)/H₂O = 10/3 | 50 ± 5 | 62.7 |
| (EtOH/DMSO = 1/1)/H₂O = 10/5 | 50 ± 5 | 33.6 |
| (EtOH/DMSO = 1/1)/H₂O = 10/10 | 50 ± 5 | 8.4 |
| (EtOH/DMSO = 1/1)/H₂O = 10/20 | 50 ± 5 | 1.4 |
| (EtOH/DMSO = 1/1)/H₂O = 10/40 | 50 ± 5 | 0.3 |

According to the solubility data of Form P in DMSO/EtOH/Water systems in Example 25, an experiment (Experiment 1 in Table 51 below) was carried out. Crude Form P was dissolved into DMSO/EtOH 3V/3V at 50° C. and then 0.6V water was charged to generate supersaturation for seeding. Afterwards, 11.4V water was dosed into seed suspension in two steps (2.4V/6 h, 9V/4 h) and the suspension was further aged for around 12 h. Solids were isolated by filtration and vacuum dried at 50° C.

Several other Experiments (Experiment 2, Experiment 3, Experiment 4, Experiment 5, Experiment 7, and Experiment 8) were performed to study the effect of seed loading, seed size and scale on particle size of final product. PSD data and polarized light microscope (PLM) images indicated that higher seed loading and smaller seed size resulted in smaller particle size of product, however, the scale effect on particle size of product was not significant.

Further, in Experiment 6, wet milling was applied to obtain even and small particles of product. In line with the experimental conditions at plant, the wet milling parameters in lab were set as follows: rotor 6F, tip speed 19.5 m/s. Finally, comparison with the particle size of product before and after wet milling indicated that wet milling with one 6F rotor is not preferred to reduce the particle size.

An additional study was performed to assess the impurity purging capability in crystallization system and this experiment was monitored by HPLC at different timing points. HPLC data showed the impurity at relative retention time (RRT) 1.08 could be reduced to <0.13% (specification criteria) with a sacrifice of yield from 95% to 70%.

Particle size data are summarized in Table 51.

TABLE 51

| Experiment | | | Particle Size Distribution (PSD) | | |
|---|---|---|---|---|---|
| Number | Objective | Conditions | D10 (μm) | D50 (μm) | D90 (μm) |
| 1 | Initial | 0.5 w/w % | 19 | 100 | 213 |
| | process | seed | | | |
| 2 | Effect of | 0.5 w/w % | 17 | 93 | 187 |
| | seed | milled seed | | | |
| 3 | loading | 3.0 w/w % | 7 | 31 | 83 |
| | | milled seed | | | |
| 4 | Effect of | 1.0 w/w % | 28 | 90 | 171 |
| | seed | large seed | | | |
| 5 | size | 1.0 w/w % | 15 | 82 | 164 |
| | | milled seed | | | |
| 6 | Wet | Before | 32 | 199 | 372 |
| | milling | milling | | | |
| | study | After | 11 | 37 | 95 |
| | | milling | | | |
| 7 | Effect of | 3 g scale | 11 | 48 | 113 |
| 8 | scale | 10 g scale | 11 | 45 | 121 |

### Example 26. Crystallization Study

The purpose of this study was to develop a crystallization process for (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine with control over solid-state form and chemical stability.

The material as received was characterized by X-Ray Powder Diffraction (XRPD), Polarized Light Microscopy (PLM), Particle Size Distribution (PSD), Thermo-Gravimetric Analysis (TGA) and Differential Scanning Calorimetry (DSC). The characterization results indicated that the starting material was crystalline and matched Form P reference patterns. The starting material showed small birefringent particles with a D90, D50, and D10 of 5.35, 3.33, and 1.99 μm, respectively. Thermal results indicated that the starting material was anhydrous with a single melting endotherm with an onset of 251.15° C.

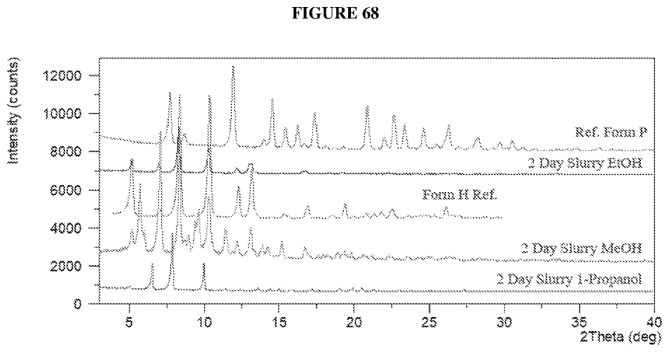

A thorough investigation was performed to evaluate the optimal solvent system for crystallization. An abbreviated polymorph/solvate screen and approximate solubility determination were performed utilizing each class III solvent and several class II solvents as listed under ICH Guidelines Q3C. In all the solvents tested, an approximate solubility of >10 mg/mL was observed in acetone, dimethyl sulfoxide (DMSO), ethanol (EtOH), N-methylpyrrolidone (NMP), methyl ethyl ketone (MEK), 2-methyl-1propanol, 2-propanol, methanol (MeOH), and tetrahydrofuran (THF). Among these solvents, form change was observed in EtOH (Form H), 1-propanol, and MeOH (Form H with additional peaks). See FIG. 68. TGA of samples obtained from EtOH and MeOH did not display a weight loss corresponding to a stoichiometric solvate whereas the 1-propanol sample showed a weight loss of 9.31%. A competitive slurry of Form P and Form H from RT to 70° C. in 2 solvents other than EtOH resulted in conversion to Form P indicating that it is the most stable form in the conditions tested. Form P was found to be more stable than Form H at 70° C. in EtOH. Form P was chosen as the target polymorph for crystallization. Throughout this study, it was observed that dissolution of any form at 50° C. will produce Form P in non-solvating solvents. Equilibrium solubility in several of these with low boiling points did not reach the target 100 mg/mL solubility at temperatures up to 70° C. The highest observed solubility with no form change was in EtOH at 56.68 mg/mL at 70° C. To reach 100 mg/mL solubility, mixtures of EtOH/DMSO were prepared and the final solvent system selected was EtOH/DMSO (80/20 v/v). In addition, this solvent system was examined at 80° C. for potential degradation and a purity of 99.9% was measured after a 24 hour slurry.

Small scale (50-100 mg) crystallization experiments found that H₂O was the most suitable anti-solvent. However, it was found that cooling followed by crystallizing with the addition of H₂O produced an amorphous powder. Crystallizing by H₂O addition at 60° C. produced Form P without the use of seeds. At the 1 gram scale, parameters such as total anti-solvent volume, rate of anti-solvent addition, seed point, and seed load were optimized individually and their impacts on particle size, resulting polymorph, filterability, residual solvent, purity, mother liquor concentration, and yield were closely monitored. Two additional 1 gram crystallizations were performed in an attempt to reduce the overall volume of solvent needed, but poor filterability and DMSO content above 5000 ppm were observed.

The optimized crystallization process was performed at the 10 gram scale. Two 10 gram crystallizations were performed, the first (Crystallization 1) as seeds and the second (Crystallization 2) using the initial 10 gram batch product as seeds.

The difference between these two experiments were the seeds used. For samples in Crystallization 1, milled Form P was used as seeds. For samples in Crystallization 2, crystals from Crystallization 1 were used as seeds to determine the variability in particle size that can be expected when seeding with larger crystals (seeding from previous batch) compared to seeding with milled material.

Both crystallizations were performed in a 300 mL jacketed ChemGlass reactor. The solvent system used was EtOH/DMSO (80/20 v/v) with a starting concentration of ˜100 mg/mL at 70° C. An anchor stir rod was used with a stir rate of 400 rpm. Once a clear solution was obtained at 70° C., 1 part (or 10 volumes) of H₂O was added. A clear solution remained. 0.5% w/w seeds were added to the solution and aged for 30 minutes. 0.5 parts (or 5 volumes) of H₂O was added to the suspension at a controlled rate of 1.2 mL/minute. Afterwards, the suspension was cooled from 70° C. to 20° C. at a rate of 10° C./hour. Once the temperature reached 20° C., vacuum filtration was utilized to isolate the solids using a 150 mL fritted filter with medium porosity. A wash of 2 parts (or 20 volumes) of H₂O was used to remove any residual organic solvents. No cracking, washing away of fines, or puck formation was observed in the dry cakes of both 10 gram samples. The solids were then transferred to a vacuum oven to tray dry at RT overnight.

In the second batch, crystals with rod morphology and a length of greater than 200 μm were observed. Two final scaled up batches were performed. A 43 gram batch was produced as small seeds and a 22 gram batch was produced using the first 10 gram batch (Crystallization 1) as large seeds to assess the impact of different size seeds on the process. Scanning electron microscopy (SEM) was used to compliment the polarized light microscope (PLM) and particle size distribution (PSD) measurements. The observation suggested that in the 22 gram batch grew initially on the seeds producing agglomerates of rods greater than 200 μm in size, followed by a primary nucleation leading to crystals less than 50 μm. This was not observed in the 43 gram batch likely because the increased surface area offered by the milled seeds provided significantly more sites for crystal growth, whereas the larger seeds used in the 22 gram batch had an overall less surface area resulting in slow de-supersaturation and primary nucleation leading to smaller particles growing on the large rods.

Conditions and results of the crystallizations are shown in Table 52.

TABLE 52

| Parameter | Crystallization 1 | | Crystallization 2 | |
|---|---|---|---|---|
| Solvent System | EtOH/DMSO | | EtOH/DMSO | |
| | (80/20 v/v) | | (80/20 v/v) | |
| Stirring Speed/Method | 400 | rpm/paddle | 400 | rpm/paddle |
| Anti-Solvent (volume added) | H₂O (15 volumes | | H₂O (15 volumes | |
| | or 1.5 parts) | | or 1.5 parts) | |
| Seeding Point, Seed Loading | 1 part or 10 volumes | | 1 part or 10 volumes | |
| (Seeds used) | H₂O addition, | | H₂O addition, | |
| | 0.5% w/w seed | | 0.5% w/w seed | |
| Seed Bed Aging Time | 30 | minutes | 30 | minutes |
| Rate Anti-Solvent Addition | 1.2 | mL/min | 1.2 | mL/min |

| after seeds added | | | |
|---|---|---|---|
| Starting Temperature, Final | | 70° C., 20° C., | 70° C., 20° C., |
| Temperature, Cooling Rate | | 10° C./hour | 10° C./hour |
| XRPD | | Form P | Form P |
| Particle Size | D(10) | 37.2 | 23.4 |
| Distribution | D(50) | 115 | 126 |
| | D(90) | 196 | 634 |

| KF Water Content % | 0.10 | | 0.16 | |
|---|---|---|---|---|
| DMSO Content % | 0.02% | | <LOD 0.0008 mg/mL | |
| Final Concentration at 20° C. | 3.55 | mg/mL | 2.22 | mg/mL |

| | | |
|---|---|---|
| (Mother Liquor (mg/mL)) | | |
| Assay Purity % | 99.9 | 100 |
| Yield % | 86.4% | 84.0% |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, anhydrous free base (Form P), characterized by a powder X-ray diffraction pattern having a characteristic peak in degrees 2θ at about 7.6.

2. The crystalline form of claim 1, characterized by a powder X-ray diffraction pattern having characteristic peaks in degrees 2θ at about 7.6, 11.9, and 15.3.

3. The crystalline form of claim 2, characterized by a powder X-ray diffraction pattern having characteristic peaks in degrees 2θ at about 7.6, 11.9, 14.5, 15.3, 20.7, and 22.6.

4. The crystalline form of claim 3, characterized by a powder X-ray diffraction pattern having characteristic peaks in degrees 2θ at about 7.6, 11.9, 14.5, 15.3, 16.1, 17.2, 17.3, 20.7, 22.6, 23.3, 26.2, and 24.5.

5. The crystalline form of any one of claims 1-4, wherein the powder X-ray diffraction pattern was obtained using Cu Kα radiation.

6. The crystalline form of any one of claims 1-5, characterized by a differential scanning calorimetry (DSC) profile having a characteristic endotherm with an onset of about 252° C. and a peak of about 253° C.

7. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form A), characterized by a powder X-ray diffraction pattern having characteristic peaks in degrees 2θ at about 5.9, 8.5, 9.3, 9.5, 10.0, 10.7, 11.7, 14.4, 18.7, 19.0, 25.8, and 28.4, wherein the powder X-ray diffraction pattern was obtained using Cu Kα radiation.

8. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form B), characterized by a powder X-ray diffraction pattern having characteristic peaks in degrees 2θ at about 5.1, 8.5, 9.4, 10.0, 10.7, 11.5, 11.7, 14.4, 15.7, 18.6, 19.8, 20.6, and 21.4, wherein the powder X-ray diffraction pattern was obtained using Cu Kα radiation.

9. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form C), characterized by a powder X-ray diffraction pattern having characteristic peaks in degrees 2θ at about 4.0, 5.3, 7.6, 9.0, 9.7, 11.2, 12.3, 12.9, 13.7, 14.5, 15.9, and 19.0, wherein the powder X-ray diffraction pattern was obtained using Cu Kα radiation.

10. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form D), characterized by a powder X-ray diffraction pattern having characteristic peaks in degrees 2θ at about 7.3, 8.6, 10.6 and 12.9, wherein the powder X-ray diffraction pattern was obtained using Cu Kα radiation.

11. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate (Form E), characterized by a powder X-ray diffraction pattern having characteristic peaks in degrees 2θ at about 4.6, 9.3, 11.6, 13.2, 13.5, 13.7, 15.1, 18.1, 18.5, 19.3, 25.7, and 27.0, wherein the powder X-ray diffraction pattern was obtained using Cu Kα radiation.

12. The crystalline form of claim 11, characterized by a differential scanning calorimetry (DSC) profile having a characteristic endotherm with an onset of about 44° C. and a peak of about 58° C., a characteristic endotherm with an onset of about 110° C. and a peak of about 114° C., and a characteristic endotherm with an onset of about 166° C. and a peak of about 177° C.

13. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form F), characterized by a powder X-ray diffraction pattern having characteristic peaks in degrees 2θ at about 4.5, 7.0, 8.2, 9.0, 9.3, 12.5, 13.5, 15.3, 17.8, 18.7, 22.6, and 26.8, wherein the powder X-ray diffraction pattern was obtained using Cu Kα radiation.

14. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base hydrate (Form H), characterized by a powder X-ray diffraction pattern having characteristic peaks in degrees 2θ at about 5.3, 7.2, 8.5, 10.4, 12.4, 13.3, 17.0, 19.5, 22.6, 24.5, 26.2, and 26.7, wherein the powder X-ray diffraction pattern was obtained using Cu Kα radiation.

15. The crystalline form of claim 14, characterized by a differential scanning calorimetry (DSC) profile having a characteristic endotherm with an onset of about 58° C. and a peak of about 84° C., a characteristic endotherm with an onset of about 63° C. and a peak of about 89° C., and a characteristic endotherm with an onset of about 169° C. and a peak of about 176° C.

16. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form I), characterized by a powder X-ray diffraction pattern having characteristic peaks in degrees 2θ at about 5.2, 7.0, 8.4, 10.4, 12.2, 13.1, 16.8, 19.2, 20.7, 22.3, and 25.9, wherein the powder X-ray diffraction pattern was obtained using Cu Kα radiation.

17. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form J), characterized by a powder X-ray diffraction pattern having characteristic peaks in degrees 2θ at about 4.1, 6.2, 8.0, 9.3, 12.8, 16.6, 18.4, 23.8, 24.4, and 28.3, wherein the powder X-ray diffraction pattern was obtained using Cu Kα radiation.

18. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form K), characterized by a powder X-ray diffraction pattern having characteristic peaks in degrees 2θ at about 6.4, 9.6, 10.5, 13.6, 19.4, 21.0, 22.4, 23.4, 23.7, 25.2, and 26.9, wherein the powder X-ray diffraction pattern was obtained using Cu Kα radiation.

19. The crystalline form of claim 18, characterized by a differential scanning calorimetry (DSC) profile having a characteristic endotherm with an onset of about 226° C. and a peak of about 230° C.

20. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form L), characterized by a powder X-ray diffraction pattern having characteristic peaks in degrees 2θ at about 6.7, 8.3, 9.1, 9.5, 11.9, 12.2, 14.3, 16.2, 20.5, 21.9, 23.1, and 25.5, wherein the powder X-ray diffraction pattern was obtained using Cu Kα radiation.

21. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form M), characterized by a powder X-ray diffraction pattern having characteristic peaks in degrees 2θ at about 6.4, 6.8, 8.3, 9.6, 12.7, 13.4, 14.3, 17.3, 19.6, 21.0, 23.9, and 26.5, wherein the powder X-ray diffraction pattern was obtained using Cu Kα radiation.

22. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form N), characterized by a powder X-ray diffraction pattern having characteristic peaks in degrees 2θ at about 5.6, 6.1, 6.9, 7.2, 8.4, 11.8, 13.7, 14.5, 17.3, 19.8, 21.9, 24.9, and 25.7, wherein the powder X-ray diffraction pattern was obtained using Cu Kα radiation.

23. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form O), characterized by a powder X-ray diffraction pattern having characteristic peaks in degrees 2θ at about 9.0, 10.6, 11.3, 14.1, 15.8, 16.3, 17.6, 19.5, 21.3, 22.7, 22.9, 25.1, and 28.7, wherein the powder X-ray diffraction pattern was obtained using Cu Kα radiation.

24. The crystalline form of claim 23, characterized by a differential scanning calorimetry (DSC) profile having a characteristic endotherm with an onset of about 166° C. and a peak of about 172° C., and a characteristic endotherm with an onset of about 196° C. and a peak of about 204° C.

25. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form Q), characterized by a powder X-ray diffraction pattern having characteristic peaks in degrees 2θ at about 4.8, 5.8, 7.7, 9.6, 11.6, 13.8, 14.7, 19.9, 21.5, 24.1, 24.7, and 27.4, wherein the powder X-ray diffraction pattern was obtained using Cu Kα radiation.

26. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form R), characterized by a powder X-ray diffraction pattern having characteristic peaks in degrees 2θ at about 7.5, 8.5, 10.1, 12.8, 14.8, 15.6, 16.9, 17.4, 18.3, 19.5, 20.0, and 21.8, wherein the powder X-ray diffraction pattern was obtained using Cu Kα radiation.

27. The crystalline form of claim 26, characterized by a differential scanning calorimetry (DSC) profile having a characteristic endotherm with an onset of about 148° C. and a peak of about 152° C., and a characteristic endotherm with an onset of about 241° C. and a peak of about 251° C.

28. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form S), characterized by a powder X-ray diffraction pattern having characteristic peaks in degrees 2θ at about 5.0, 5.9, 8.0, 9.7, and 11.8, wherein the powder X-ray diffraction pattern was obtained using Cu Kα radiation.

29. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form T), characterized by a powder X-ray diffraction pattern having characteristic peaks in degrees 2θ at about 7.0, 8.7, 10.3, 11.8, 14.1, 17.0, 20.9, 22.4, 24.5, 25.5, and 27.9, wherein the powder X-ray diffraction pattern was obtained using Cu Kα radiation.

30. The crystalline form of claim 29, characterized by a differential scanning calorimetry (DSC) profile having a characteristic endotherm with an onset of about 83° C. and a peak of about 84° C., and a characteristic endotherm with an onset of about 249° C. and a peak of about 251° C.

31. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form U), characterized by a powder X-ray diffraction pattern having characteristic peaks in degrees 2θ at about 6.2, 6.9, 7.6, 9.0, 10.5, 12.3, and 14.5, wherein the powder X-ray diffraction pattern was obtained using Cu Kα radiation.

32. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form V), characterized by a powder X-ray diffraction pattern having characteristic peaks in degrees 2θ at about 6.0, 8.0, 10.8, 12.0, 15.6, 15.9, 16.8, 18.3, 19.7, 20.9, 21.0, and 26.3, wherein the powder X-ray diffraction pattern was obtained using Cu Kα radiation.

33. A crystalline form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base (Form W), characterized by a powder X-ray diffraction pattern having characteristic peaks in degrees 2θ at about 4.3, 8.6, 12.9, 15.2, 23.3, and 26.0, wherein the powder X-ray diffraction pattern was obtained using Cu Kα radiation.

34. A substantially amorphous form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, free base.

35. A pharmaceutical composition comprising a crystalline form of any one of claims 1-33 or the amorphous form of claim 34, and a pharmaceutically acceptable excipient.

36. A pharmaceutical composition comprising the crystalline Form P of any one of claims 1-6, and a pharmaceutically acceptable excipient.

37. A pharmaceutical composition formed from the crystalline Form P of any one of claims 1-6.

38. The pharmaceutical composition of any one of claims 35-37, wherein the composition is a formulation for oral administration.

39. A drug substance comprising at least a detectable amount of the crystalline form of any one of claims 1-33.

40. A drug substance comprising a substantially pure crystalline form of any one of claims 1-33.

41. A drug substance comprising a substantially pure crystalline Form P of any one of claims 1-6.

42. A pharmaceutically acceptable salt of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, wherein the salt is selected from the group consisting of a benzenesulfonic acid salt, a citric acid salt, a fumaric acid salt, a maleic acid salt, a L-malic acid salt, a methanesulfonic acid salt, a phosphoric acid salt, a pyruvic acid salt, a sulfuric acid salt, a L-tartaric acid salt, a toluenesulfonic acid salt, and hydrates and solvates thereof.

43. A pharmaceutically acceptable, crystalline salt form of (S)-12-fluoro-4-(2-methylpyridin-3-yl)-7a,8,13,14-tetrahydro-7H-[1,2,4]triazolo[4′,3′:1,6]pyrido[3,2-b]benzofuro[4,3-fg][1,4]oxazonine, wherein the crystalline salt is selected from the group consisting of a benzenesulfonic acid salt, a citric acid salt, a fumaric acid salt, a hydrochloride salt, a maleic acid salt, a L-malic acid salt, a methanesulfonic acid salt, a phosphoric acid salt, a pyruvic acid salt, a sulfuric acid salt, a L-tartaric acid salt, and a toluenesulfonic acid salt, and crystalline hydrates and solvates thereof.

44. The benzenesulfonic acid salt of claim 43, wherein the benzenesulfonic acid salt is a crystalline form (Form 1-A) characterized by a powder X-ray diffraction pattern having characteristic peaks in degrees 2θ at about 5.1, 6.5, 6.8, 7.0, 9.6, 12.1, 14.8, 15.1, 15.5, 16.4, 18.6, and 21.3, wherein the powder X-ray diffraction pattern was obtained using Cu Kα radiation.

45. The benzenesulfonic acid salt of claim 43, wherein the benzenesulfonic acid salt is a crystalline form (Form 1-B) characterized by a powder X-ray diffraction pattern having characteristic peaks in degrees 2θ at about 5.3, 6.2, 6.7, 8,2, 10.0, 10.6, 12.3, 14.0, 15.6, 16.0, 16.2, and 22.1, wherein the powder X-ray diffraction pattern was obtained using Cu Kα radiation.

46. The crystalline form of claim 45, characterized by a differential scanning calorimetry (DSC) profile having a characteristic endotherm with an onset of about 33° C. and a peak of about 71° C., a characteristic endotherm with an onset of about 120° C. and a peak of about 133° C., and a characteristic endotherm with an onset of about 154° C. and a peak of about 159° C.

47. The citric acid salt of claim 43, wherein the citric acid salt is a crystalline form (Form 2-A) characterized by a powder X-ray diffraction pattern having characteristic peaks in degrees 2θ at about 4.7, 7.0, 7.9, 9.1, 10.2, 11.4, 12.5, 13.7, 14.1, 15.1, 18.3, and 19.0, wherein the powder X-ray diffraction pattern was obtained using Cu Kα radiation.

48. The citric acid salt of claim 43, wherein the citric acid salt is a crystalline form (Form 2-B) characterized by a powder X-ray diffraction pattern having characteristic peaks in degrees 2θ at about 6.7, 7.9, 9.3, 11.2, 12.5, 13.8, 15.2, 17.0, 18.4, 19.5, 22.3, and 28.5, wherein the powder X-ray diffraction pattern was obtained using Cu Kα radiation.

49. The citric acid salt of claim 43, wherein the citric acid salt is a crystalline form (Form 2-C) characterized by a powder X-ray diffraction pattern having characteristic peaks in degrees 2θ at about 6.7, 7.9, 9.1, 12.5, 13.6, 14.3, 15.0, 16.9, 18.2, 19.2, 22.1, and 29.0, wherein the powder X-ray diffraction pattern was obtained using Cu Kα radiation.

50. The crystalline form of claim 49, characterized by a differential scanning calorimetry (DSC) profile having a characteristic endotherm with an onset of about 33° C. and a peak of about 60° C., a characteristic endotherm with an onset of about 96° C. and a peak of about 116° C., a characteristic endotherm with an onset of about 160° C. and a peak of about 169° C., and a characteristic endotherm with an onset of about 141° C. and a peak of about 177° C.

51. The fumaric acid salt of claim 43, wherein the fumaric acid salt is a crystalline form (Form 3-A) characterized by a powder X-ray diffraction pattern having characteristic peaks in degrees 2θ at about 7.1, 8.2, 14.3, 16.5, 18.0, 18.9, 21.9, 22.1, 22.4, 24.5, 24.8, and 25.4, wherein the powder X-ray diffraction pattern was obtained using Cu Kα radiation.

52. The crystalline form of claim 51, characterized by a differential scanning calorimetry (DSC) profile having a characteristic endotherm with an onset of about 237° C. and a peak of about 241° C.

53. The hydrochloride salt of claim 43, wherein the hydrochloride salt is a crystalline form (Form 5-A) characterized by a powder X-ray diffraction pattern having characteristic peaks in degrees 2θ at about 5.0, 7.8, 8.1, 9.3, 12.2, 13.5, 13.9, 14.1, 19.3, 20.3, 21.3 and 25.2, wherein the powder X-ray diffraction pattern was obtained using Cu Kα radiation.

54. The hydrochloride salt of claim 43, wherein the hydrochloride salt is a crystalline form (Form 5-B) characterized by a powder X-ray diffraction pattern having characteristic peaks in degrees 2θ at about 7.4, 9.1, 9.9, 11.7, 14.2, 17.5, 20.0, 21.2, 21.8, 23.9, 25.7, and 27.6, wherein the powder X-ray diffraction pattern was obtained using Cu Kα radiation.

55. The crystalline form of claim 54, characterized by a differential scanning calorimetry (DSC) profile having a characteristic endotherm with an onset of about 68° C. and a peak of about 82° C., a characteristic endotherm with an onset of about 111° C. and a peak of about 130° C., and a characteristic endotherm with an onset of about 193° C. and a peak of about 211° C.

56. The hydrochloride salt of claim 43, wherein the hydrochloride salt is a crystalline form (Form 5-C) characterized by a powder X-ray diffraction pattern having characteristic peaks in degrees 2θ at about 10.7, 11.8, 12.9, 16.2, 18.1, 20.5, 21.5, 22.4, 23.9, 26.4, 27.0, and 28.9, wherein the powder X-ray diffraction pattern was obtained using Cu Kα radiation.

57. The crystalline form of claim 56, characterized by a differential scanning calorimetry (DSC) profile having a characteristic endotherm with an onset of about 140° C. and a peak of about 145° C., and a characteristic endotherm with an onset of about 213° C. and a peak of about 230° C.

58. The hydrochloride salt of claim 43, wherein the hydrochloride salt is a crystalline form (Form 5-D) characterized by a powder X-ray diffraction pattern having characteristic peaks in degrees 2θ at about 7.8, 8.4, 12.7, 14.0, 15.0, 17.0, 22.9, 25.6, and 26.0, wherein the powder X-ray diffraction pattern was obtained using Cu Kα radiation.

59. The maleic acid salt of claim 43, wherein the maleic acid salt is a crystalline form (Form 7-A) characterized by a powder X-ray diffraction pattern having characteristic peaks in degrees 2θ at about 7.2, 8.3, 14.3, 14.9, 16.5, 18.9, 21.5, 22.7, 25.3, 25.9, 27.2, and 27.5, wherein the powder X-ray diffraction pattern was obtained using Cu Kα radiation.

60. The crystalline form of claim 59, characterized by a differential scanning calorimetry (DSC) profile having a characteristic endotherm with an onset of about 215° C. and a peak of about 221° C., and a characteristic endotherm with an onset of about 216° C. and a peak of about 225° C.

61. The L-malic acid salt of claim 43, wherein the L-malic acid salt is a crystalline form (Form 8-A) characterized by a powder X-ray diffraction pattern having characteristic peaks in degrees 2θ at about 7.3, 8.3, 11.0, 14.3, 15.0, 16.6, 19.0, 21.6, 22.0, 24.7, 25.5, and 27.3, wherein the powder X-ray diffraction pattern was obtained using Cu Kα radiation.

62. The crystalline form of claim 61, characterized by a differential scanning calorimetry (DSC) profile having a characteristic endotherm with an onset of about 177° C. and a peak of about 201° C., a characteristic endotherm with an onset of about 186° C. and a peak of about 207° C., a characteristic endotherm with an onset of about 205° C. and a peak of about 211° C., and a characteristic endotherm with an onset of about 208° C. and a peak of about 216° C.

63. The L-malic acid salt of claim 43, wherein the L-malic acid salt is a crystalline form (Form 8-B) characterized by a powder X-ray diffraction pattern having characteristic peaks in degrees 2θ at about 5.9, 9.1, 11.6, 16.3, 17.6, 18.2, 19.1, 21.2, 22.9, 23.8, 27.1, and 28.4, wherein the powder X-ray diffraction pattern was obtained using Cu Kα radiation.

64. The crystalline form of claim 63, characterized by a differential scanning calorimetry (DSC) profile having a characteristic endotherm with an onset of about 189° C. and a peak of about 192° C., and a characteristic endotherm with an onset of about 186° C. and a peak of about 202° C.

65. The methanesulfonic acid salt of claim 43, wherein the methanesulfonic acid salt is a crystalline form (Form 9-A) characterized by a powder X-ray diffraction pattern having characteristic peaks in degrees 2θ at about 4.5, 5.4, 7.5, 9.1, 9.5, 10.1, 14.7, 15.0, 15.7, 18.0, 20.1, and 21.5, wherein the powder X-ray diffraction pattern was obtained using Cu Kα radiation.

66. The methanesulfonic acid salt of claim 43, wherein the methanesulfonic acid salt is a crystalline form (Form 9-B) characterized by a powder X-ray diffraction pattern having characteristic peaks in degrees 2θ at about 8.2, 8.7, 13.7, 14.7, 16.2, 18.9, 22.3, 22.8, 24.4, 27.0, 27.5, and 28.9, wherein the powder X-ray diffraction pattern was obtained using Cu Kα radiation.

67. The crystalline form of claim 66, characterized by a differential scanning calorimetry (DSC) profile having a characteristic endotherm with an onset of about 51° C. and a peak of about 74° C., and a characteristic endotherm with an onset of about 177° C. and a peak of about 187° C.

68. The methanesulfonic acid salt of claim 43, wherein the methanesulfonic acid salt is a crystalline form (Form 9-C) characterized by a powder X-ray diffraction pattern having characteristic peaks in degrees 2θ at about 4.5, 7.6, 9.2, 9.5, 10.2, 10.9, 14.8, 15.0, 15.9, 18.2, 20.4, and 21.8, wherein the powder X-ray diffraction pattern was obtained using Cu Kα radiation.

69. The phosphoric acid salt of claim 43, wherein the phosphoric acid salt is a crystalline form (Form 10-A) characterized by a powder X-ray diffraction pattern having characteristic peaks in degrees 2θ at about 6.2, 8.5, 10.2, 10.8, 11.5, 12.3, 15.7, 18.4, 20.5, 21.2, 22.9, and 25.8, wherein the powder X-ray diffraction pattern was obtained using Cu Kα radiation.

70. The crystalline form of claim 69, characterized by a differential scanning calorimetry (DSC) profile having a characteristic endotherm with an onset of about 282° C. and a peak of about 290° C., and a characteristic endotherm with an onset of about 283° C. and a peak of about 294° C.

71. The pyruvic acid salt of claim 43, wherein the pyruvic acid salt is a crystalline form (Form 11-A) characterized by a powder X-ray diffraction pattern having characteristic peaks in degrees 2θ at about 4.4, 4.9, 7.2, 7.5, 8.6, 9.1, 9.9, 11.4, 13.1, 14.5, 14.7, and 17.2, wherein the powder X-ray diffraction pattern was obtained using Cu Kα radiation.

72. The crystalline form of claim 71, characterized by a differential scanning calorimetry (DSC) profile having a characteristic endotherm with an onset of about 76° C. and a peak of about 88° C., a characteristic endotherm with an onset of about 134° C. and a peak of about 142° C., and a characteristic endotherm with an onset of about 149° C. and a peak of about 157° C.

73. The sulfuric acid salt of claim 43, wherein the sulfuric acid salt is a crystalline form (Form 12-A) characterized by a powder X-ray diffraction pattern having characteristic peaks in degrees 2θ at about 5.9, 6.6, 7.9, 8.2, 9.6, 11.8, 14.7, 15.3, 17.1, 17.7, 18.9, and 22.6, wherein the powder X-ray diffraction pattern was obtained using Cu Kα radiation.

74. The crystalline form of claim 73, characterized by a differential scanning calorimetry (DSC) profile having a characteristic endotherm with an onset of about 48° C. and a peak of about 81° C., a characteristic endotherm with an onset of about 169° C. and a peak of about 185° C., and a characteristic endotherm with an onset of about 229° C. and a peak of about 241° C.

75. The L-tartaric acid salt of claim 43, wherein the L-tartaric acid salt is a crystalline form (Form 13-A) characterized by a powder X-ray diffraction pattern having characteristic peaks in degrees 2θ at about 8.0, 8.7, 11.1, 14.2, 15.9, 17.3, 19.5, 21.6, 23.1, 23.6, 26.9, and 28.2, wherein the powder X-ray diffraction pattern was obtained using Cu Kα radiation.

76. The crystalline form of claim 75, characterized by a differential scanning calorimetry (DSC) profile having a characteristic endotherm with an onset of about 213° C. and a peak of about 222° C.

77. The L-tartaric acid salt of claim 43, wherein the L-tartaric acid salt is a crystalline form (Form 13-B) characterized by a powder X-ray diffraction pattern having characteristic peaks in degrees 2θ at about 7.1, 8.7, 11.8, 16.9, 20.8, 21.8, 22.2, 24.1, 25.1, 25.3, 27.0, and 27.7, wherein the powder X-ray diffraction pattern was obtained using Cu Kα radiation.

78. The crystalline form of claim 77, characterized by a differential scanning calorimetry (DSC) profile having a characteristic endotherm with an onset of about 89° C. and a peak of about 115° C., a characteristic endotherm with an onset of about 157° C. and a peak of about 167° C., and a characteristic endotherm with an onset of about 181° C. and a peak of about 195° C.

79. The toluenesulfonic acid salt of claim 43, wherein the toluenesulfonic acid salt is a crystalline form (Form 14-A) characterized by a powder X-ray diffraction pattern having characteristic peaks in degrees 2θ at about 4.4, 6.1, 6.4, 6.7, 8.8, 9.5, 10.3, 15.3, 17.5, 21.3, 21.9, and 27.2, wherein the powder X-ray diffraction pattern was obtained using Cu Kα radiation.

80. The toluenesulfonic acid salt of claim 43, wherein the toluenesulfonic acid salt is a crystalline form (Form 14-B) characterized by a powder X-ray diffraction pattern having characteristic peaks in degrees 2θ at about 6.8, 7.7, 12.9, 15.3, 17.2, 21.4, 22.4, 24.7, and 25.9, wherein the powder X-ray diffraction pattern was obtained using Cu Kα radiation.

81. A pharmaceutical composition comprising a crystalline salt form of any one of claims 43-80, and a pharmaceutically acceptable excipient.

82. A pharmaceutical composition formed from the crystalline salt form of any one of claims 43-80.

83. A drug substance comprising at least a detectable amount of the crystalline salt form of any one of claims 43-80.

84. A drug substance comprising a substantially pure crystalline salt form of any one of claims 43-80.
